(19) United States
(12) Patent Application Publication
(10) Pub. No.: US 2026/0271502 A1
(43) Pub. Date: Sep. 10, 2026

(54) COMPOUND FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si, Gyeonggi-do (KR)

(72) Inventors: Seungjae LEE, Suwon-si, Gyeonggi-do (KR); Bowon CHOI, Suwon-si, Gyeonggi-do (KR); Hanill LEE, Suwon-si, Gyeonggi-do (KR); Yoonman LEE, Suwon-si, Gyeonggi-do (KR); Byungku KIM, Suwon-si, Gyeonggi-do (KR); Wook KIM, Suwon-si, Gyeonggi-do (KR); Seonyeong GWAK, Suwon-si, Gyeonggi-do (KR); Suheon IM, Suwon-si, Gyeonggi-do (KR); Sung-Hyun JUNG, Suwon-si, Gyeonggi-do (KR); Hyung Sun KIM, Suwon-si, Gyeonggi-do (KR); Mijin LEE, Suwon-si, Gyeonggi-do (KR)

(21) Appl. No.: 19/154,562

(22) PCT Filed: Feb. 16, 2024
(86) PCT No.: PCT/KR2024/095257

§ 371 Date: Aug. 7, 2025

(30) Foreign Application Priority Data
Feb. 23, 2023 (KR) 10-2023-0024475

(51) Int. Cl. H10K 50/12; H10K 85/30; H10K 85/40; H10K 85/60

(52) U.S. Cl. CPC H10K 50/12; H10K 85/342; H10K 85/40; H10K 85/622; H10K 85/623; H10K 85/626; H10K 85/633; H10K 85/636; H10K 85/654; H10K 85/6572; H10K 85/6574; H10K 85/6576

(57) ABSTRACT

Disclosed are a compound for an organic optoelectronic device represented by Chemical Formula 1, an organic optoelectronic device including the same, and a display device.

### TECHNICAL FIELD

A compound for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a compound for an organic optoelectronic device capable of implementing a high-efficiency and long life-span organic optoelectronic device.

Another embodiment provides an organic optoelectronic device including the compound for the organic optoelectronic device.

Another embodiment provides a display device including the organic optoelectronic device.

### Technical Solution

According to an embodiment, a compound for an organic optoelectronic device represented by Chemical Formula 1 is provided.

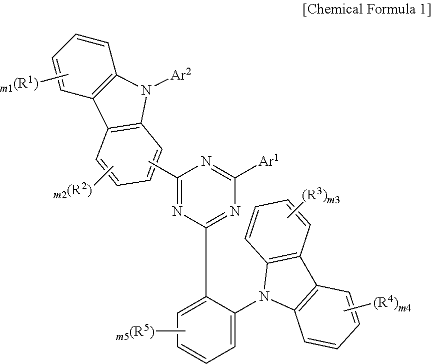

In Chemical Formula 1,
Ar¹ and Ar² are each independently a substituted or unsubstituted C6 to C20 aryl group or a substituted or unsubstituted C2 to C20 heterocyclic group,
R¹ to R⁵ are each independently hydrogen, deuterium, a cyano group, a substituted or unsubstituted C1 to C10 alkyl group, or a substituted or unsubstituted C6 to C12 aryl group,
m1, and m3 to m5 are each independently one of integers of 1 to 4,
m2 is one of integers of 1 to 3, and
Chemical Formula 1 satisfies at least one of the following (i) to (iii).
(i) at least one of R¹ to R⁵ is deuterium, a C1 to C10 alkyl group substituted with one or more deuteriums, or a C6 to C12 aryl group substituted with one or more deuteriums;
(ii) Ar¹ is a C6 to C20 aryl group substituted with one or more deuteriums or a C2 to C30 heterocyclic group substituted with one or more deuteriums; and
(iii) Ar² is a C6 to C20 aryl group substituted with one or more deuteriums or a C2 to C30 heterocyclic group substituted with one or more deuteriums.

According to another embodiment, an organic optoelectronic device includes an anode and a cathode facing each other, and at least one organic layer between the anode and the cathode, wherein the organic layer includes the compound for the organic optoelectronic device.

According to another embodiment, a display device including the organic optoelectronic device is provided.

### Advantageous Effects

An organic optoelectronic device having high efficiency and a long life-span may be realized.

### DESCRIPTION OF THE DRAWINGS

FIG. 1 is a cross-sectional view illustrating an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### BEST MODE

Hereinafter, embodiments of the present invention are described in detail. However, these embodiments are exemplary, and this disclosure is not limited thereto.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present invention, the “substituted” refers to replacement of at least one hydrogen of a substituent or a compound by deuterium, a C1 to C20 alkyl group, a C6 to C30 aryl group, or a cyano group. In specific example of the present invention, the “substituted” refers to replacement of at least one hydrogen of a substituent or a compound by deuterium, a C1 to C5 alkyl group, a C6 to C18 aryl group, or a cyano group. In specific example of the present invention, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

In the present specification, “unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quaterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

As an example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electronic characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

The compound for the organic optoelectronic device according to an embodiment may be represented by Chemical Formula 1.

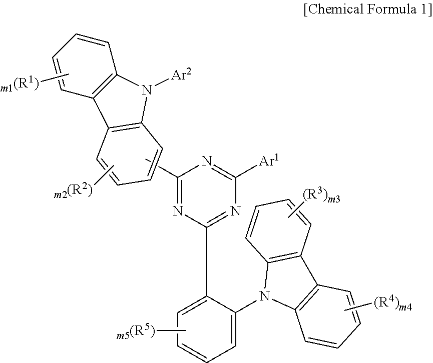

In Chemical Formula 1,
Ar¹ and Ar² are each independently a substituted or unsubstituted C6 to C20 aryl group or a substituted or unsubstituted C2 to C20 heterocyclic group,
R¹ to R⁵ are each independently hydrogen, deuterium, a cyano group, a substituted or unsubstituted C1 to C10 alkyl group, or a substituted or unsubstituted C6 to C12 aryl group,
m1, and m3 to m5 are each independently one of integers of 1 to 4,
m2 is one of integers of 1 to 3,
Chemical Formula 1 satisfies at least one of the following (i) to (iii).
(i) at least one of R¹ to R⁵ is deuterium, a C1 to C10 alkyl group substituted with one or more deuteriums, or a C6 to C12 aryl group substituted with one or more deuteriums;
(ii) Ar¹ is a C6 to C20 aryl group substituted with one or more deuteriums or a C2 to C30 heterocyclic group substituted with one or more deuteriums;
(iii) Ar² is a C6 to C20 aryl group substituted with one or more deuteriums or a C2 to C30 heterocyclic group substituted with one or more deuteriums

The compound for the organic optoelectronic device represented by Chemical Formula 1 has a structure in which triazine is substituted with two or more carbazolyl groups, and the triazine is substituted with at least one of the carbazolyl groups on at 9th position via ortho-phenylene.

As such, the first compound has a structure including ortho-carbazole in addition to the triazine, and thus has particularly excellent energy transfer efficiency as a phosphorescent dopant, and thus may be used as an advantageous material as a phosphorescent host. Substitution of the triazine with two or more carbazolyl groups helps expand a resonance structure of the carbazole moiety when applied as a host, which helps stabilize the hole transport part within the molecule. This results in an optimization of charge balance between the electron transport and hole transport parts within the molecule, which further helps to improve the life-span.

In addition, when carbazole is substituted at the ortho position of the triazine, the dihedral angle increases due to the steric hindrance between the triazine and carbazole, and the triazine moiety and the carbazole moiety twist with each other, thereby increasing the dihedral angle. This means that the electron clouds of the HOMO level and LUMO level are mostly separated without overlapping, and because it has a small ΔEst, rapid energy transfer is enabled, and it exhibits high efficiency characteristics, especially when applied as a phosphorescent host. In addition, the side reaction path in the excited state is reduced, and thus the life-span increase effect may be further obtained.

Additionally, the zero-point energy and vibrational energy of the compound may be further lowered by including at least one deuterium. This further lowers the energy of the ground state, weakens the interaction between molecules, and makes it possible to make the film formed from it into an amorphous state, which improves heat resistance and is effective in extending the life-span. That is, when applied, high-efficiency, especially long life-span organic light emitting diodes may be provided.

When m1 is 2 or more, each R¹ may be the same or different from each other.

When m2 is 2 or more, each R² may be the same or different from each other.

When m3 is 2 or more, each R³ may be the same or different from each other.

When m4 is 2 or more, each R⁴ may be the same or different from each other.

When m5 is 2 or more, each R⁵ may be the same or different from each other.

For example, at least one of R¹ to R⁵ may be deuterium, a C1 to C10 alkyl group substituted with one or more deuteriums, or a C6 to C12 aryl group substituted with one or more deuteriums, and
Ar¹ may be a C6 to C20 aryl group substituted with one or more deuteriums or a C2 to C30 heterocyclic group substituted with one or more deuteriums.

As another example, at least one of R¹ to R⁵ may be deuterium, a C1 to C10 alkyl group substituted with one or more deuteriums, or a C6 to C12 aryl group substituted with one or more deuteriums, and
Ar² may be a C6 to C20 aryl group substituted with one or more deuteriums or a C2 to C30 heterocyclic group substituted with one or more deuteriums.

As another example, at least one of R¹ to R⁵ may be deuterium, a C1 to C10 alkyl group substituted with one or more deuteriums, or a C6 to C12 aryl group substituted with one or more deuteriums,
Ar¹ may be a C6 to C20 aryl group substituted with one or more deuteriums or a C2 to C30 heterocyclic group substituted with one or more deuteriums, and
Ar² may be a C6 to C20 aryl group substituted with one or more deuteriums or a C2 to C30 heterocyclic group substituted with one or more deuteriums.

In an embodiment, a deuterium substitution ratio of Chemical Formula 1 may be 30% to 100%.

The deuterium substitution ratio refers to a percentage of deuterium substitution ratio with respect to the total number of hydrogens.

For example, if 8 out of 10 hydrogen positions are replaced by deuterium, the deuterium substitution ratio may be 80%.

For example, Chemical Formula 1 may be represented by any one of Chemical Formula 1A to Chemical Formula 1D.

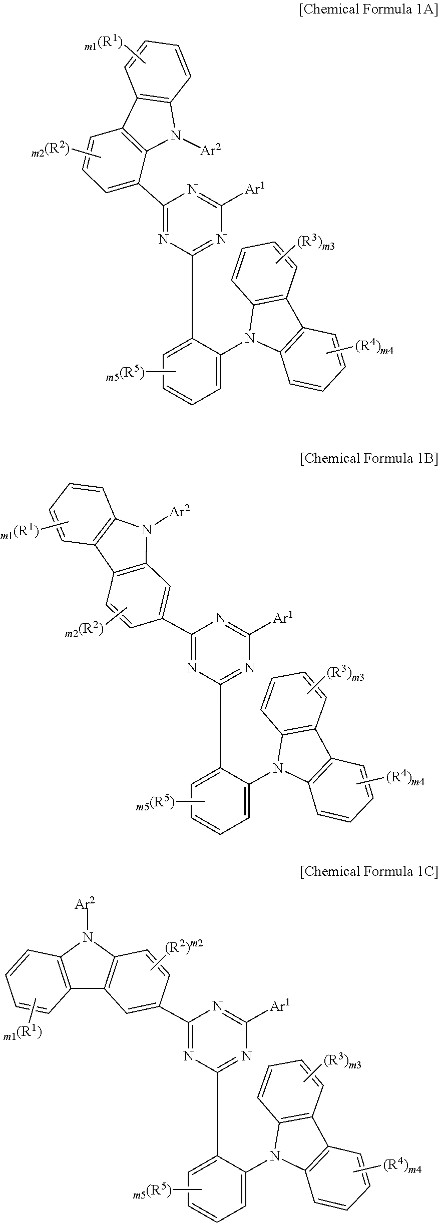

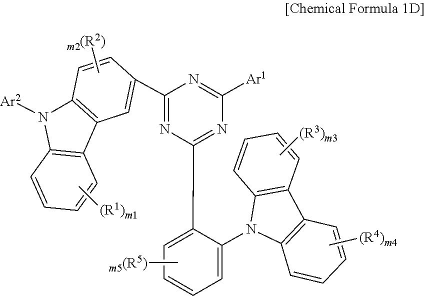

In Chemical Formula 1A to Chemical Formula 1D,
the definitions of R¹ to R⁵, Ar¹, Ar², and m1 to m5 are the same as described above.

For example, in Chemical Formula 1, Ar¹ may be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted fluorenyl group, a substituted or unsubstituted dibenzofuranyl group, a substituted or unsubstituted dibenzothiophenyl group, or a substituted or unsubstituted dibenzosilolyl group.

For example, in Chemical Formula 1, Ar² may be a substituted or unsubstituted phenyl group, a substituted or unsubstituted biphenyl group, or a substituted or unsubstituted naphthyl group.

For example, in Chemical Formula 1, R¹ to R⁵ may each independently be hydrogen, deuterium, a cyano group, a substituted or unsubstituted C1 to C5 alkyl group, a substituted or unsubstituted phenyl group, or a substituted or unsubstituted biphenyl group.

In this case, the chemical formula 1 satisfies at least one of the following (iv) to (vi).
(iv) at least one of R¹ to R⁵ is deuterium, a C1 to C5 alkyl group substituted with one or more deuteriums, a phenyl group substituted with one or more deuteriums, or a biphenyl group substituted with one or more deuteriums;
(v) Ar¹ is a phenyl group substituted with one or more deuteriums, a biphenyl group substituted with one or more deuteriums, a terphenyl group substituted with one or more deuteriums, a naphthyl group substituted with one or more deuteriums, an anthracenyl group substituted with one or more deuteriums, a phenanthrenyl group substituted with one or more deuteriums, a fluorenyl group substituted with one or more deuteriums, a dibenzofuranyl group substituted with one or more deuteriums, a dibenzothiophenyl group substituted with one or more deuteriums, or a dibenzosilolyl group substituted with one or more deuteriums; and
(vi) Ar² is a phenyl group substituted with one or more deuteriums, a biphenyl group substituted with one or more deuteriums or a naphthyl group substituted with one or more deuteriums.

As a specific example, Chemical Formula 1 may be represented by Chemical Formula 1B or Chemical Formula 1C.

For example, the first compound represented by the chemical formula 1 may be one selected from the compounds listed in Group 1, but is not limited thereto.

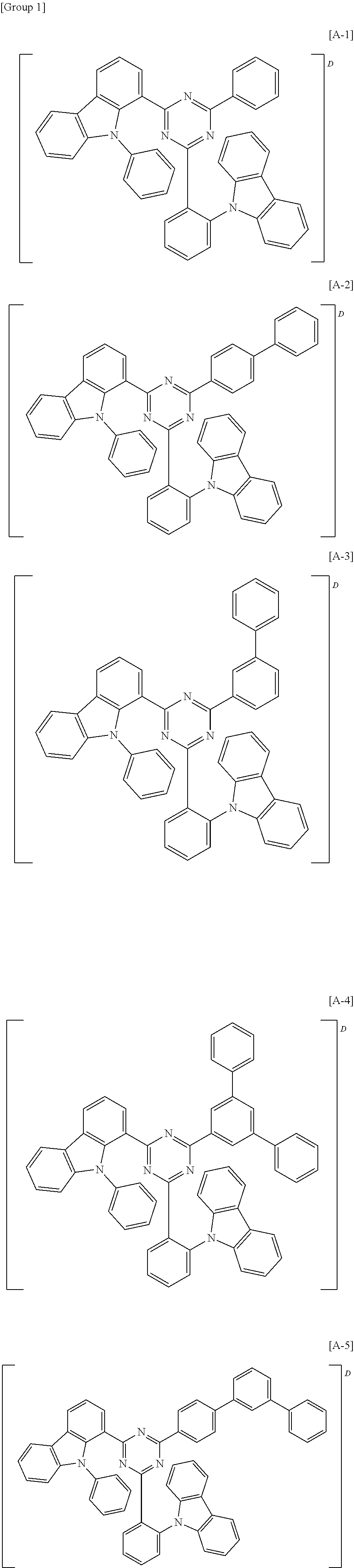

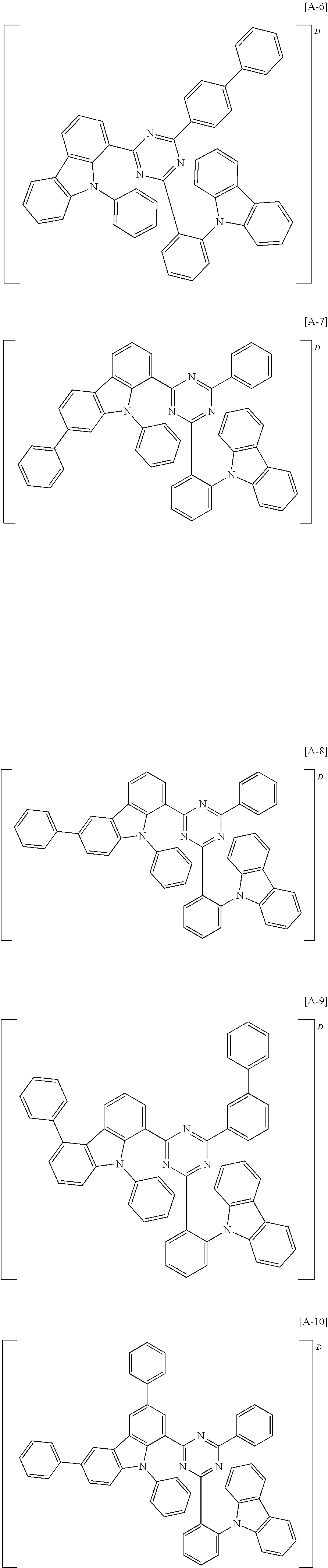

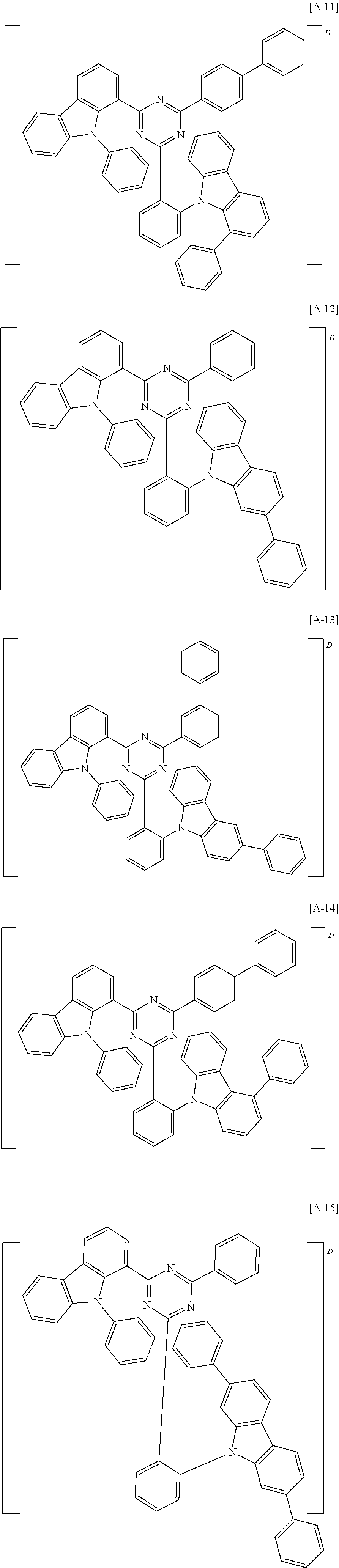

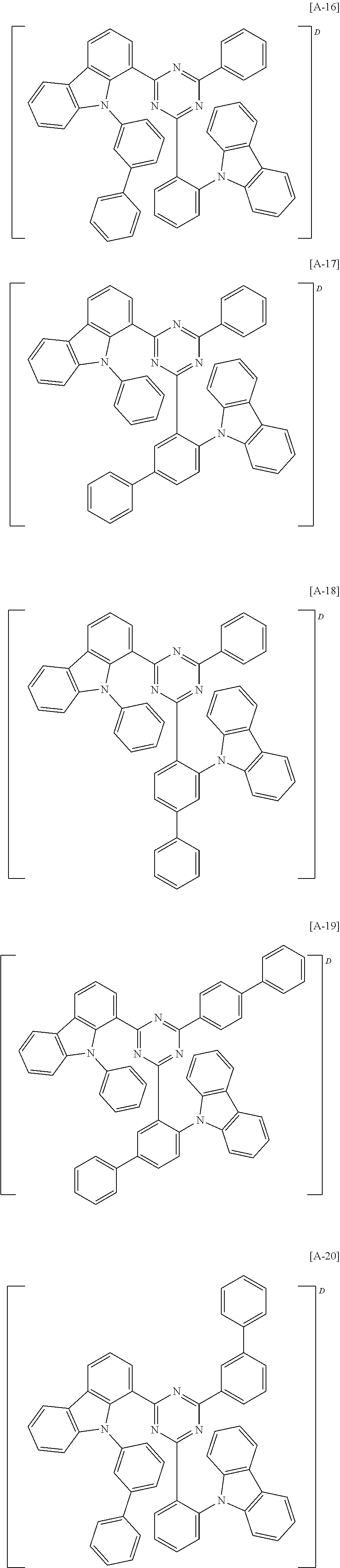

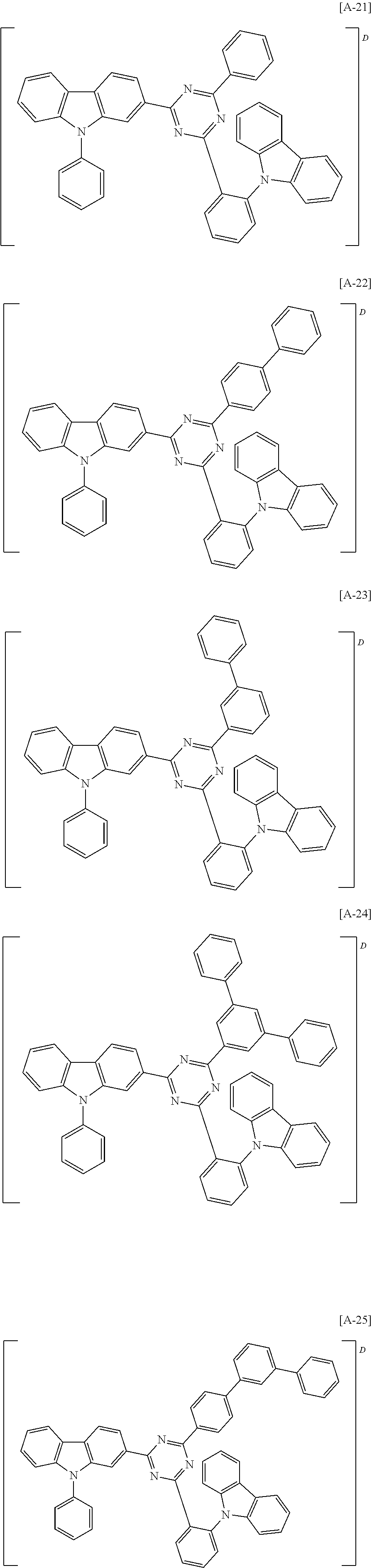

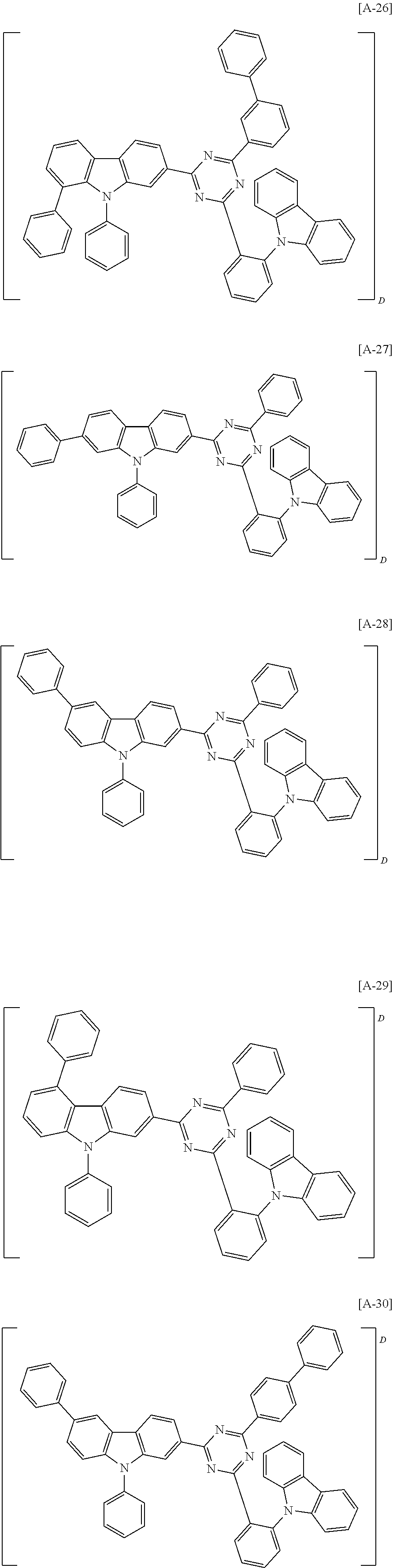

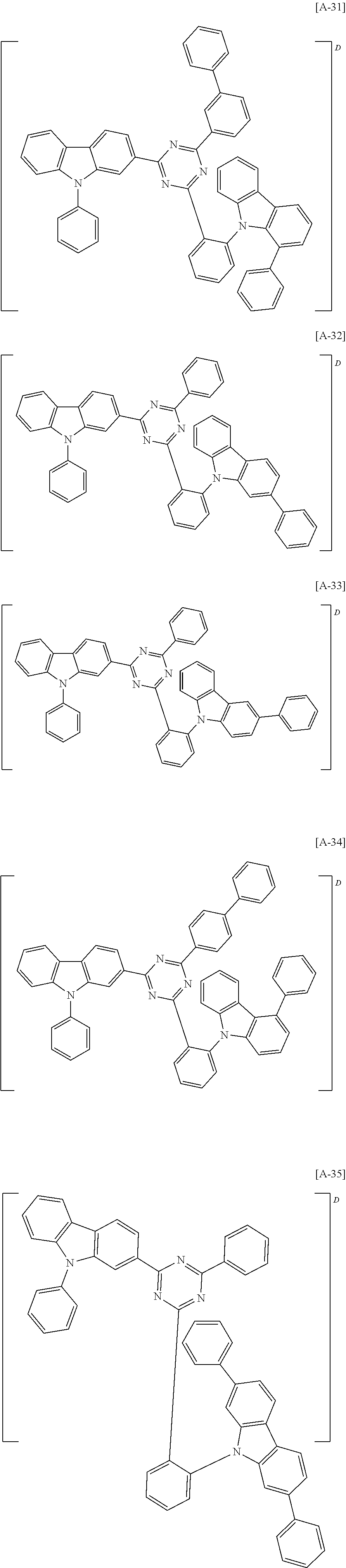

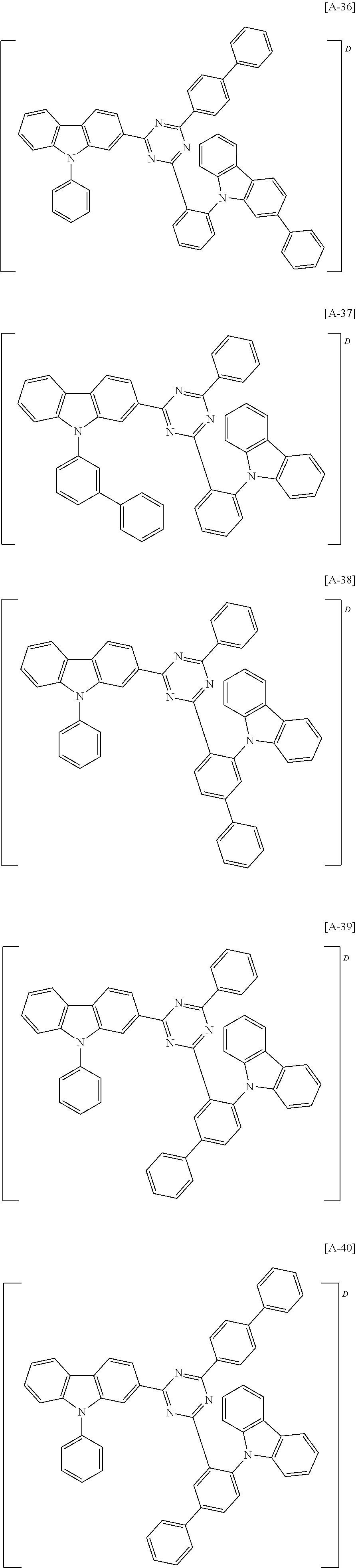

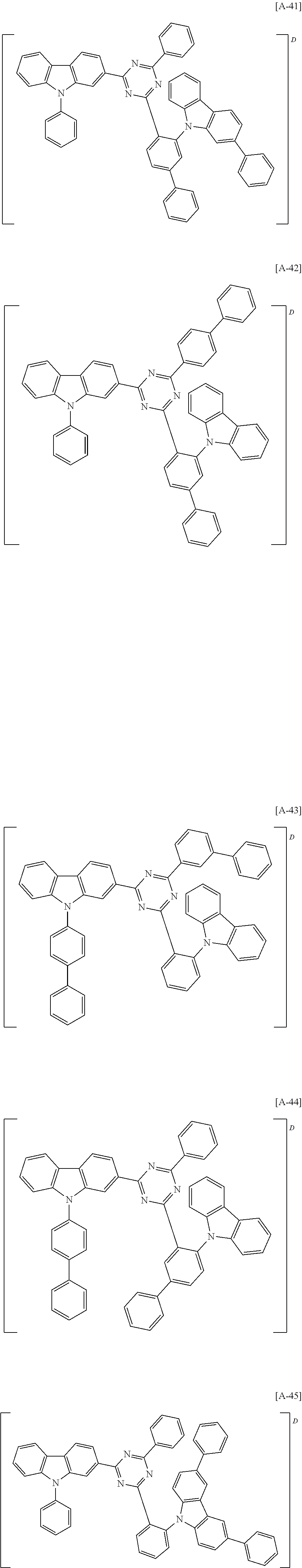

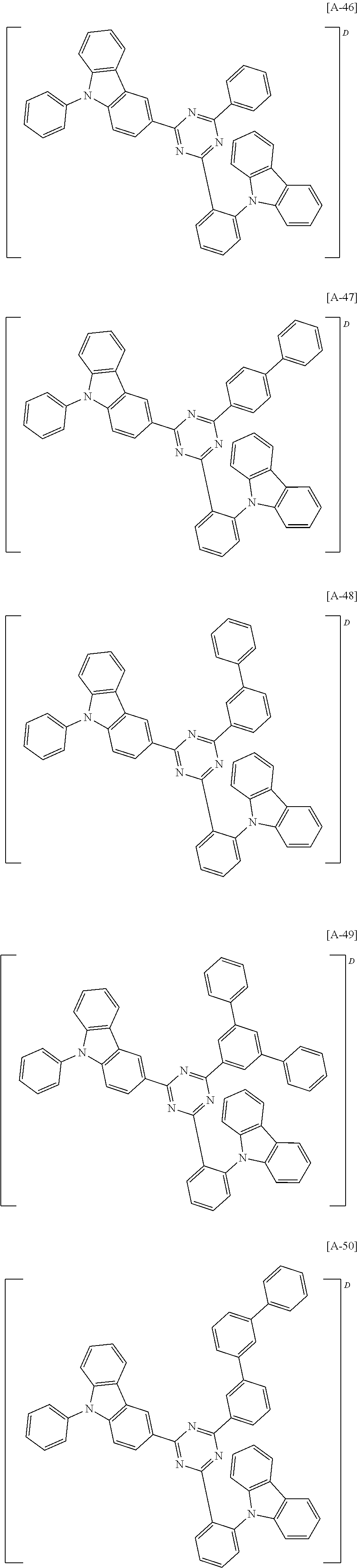

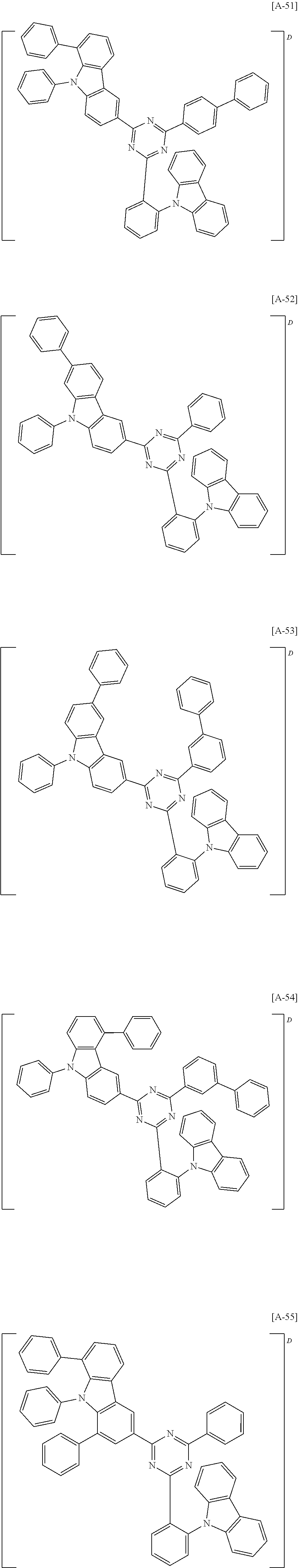

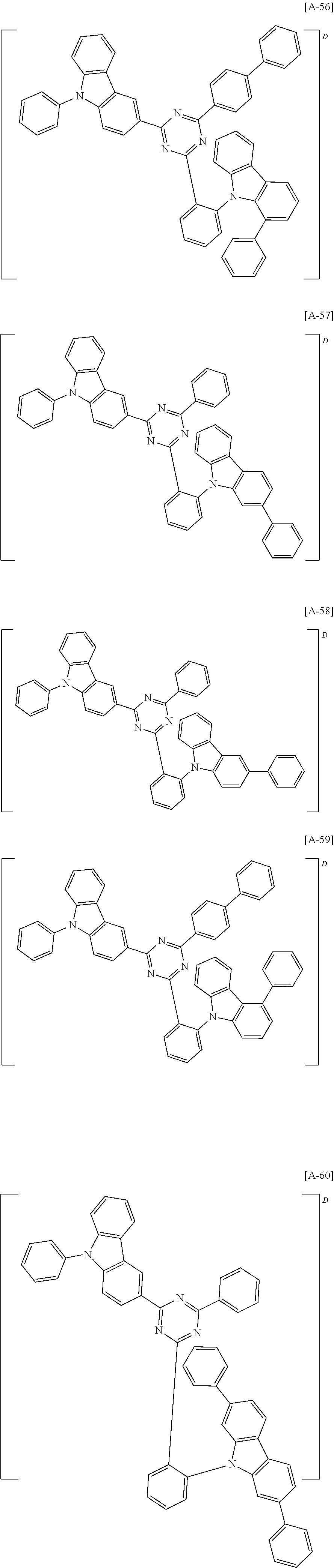

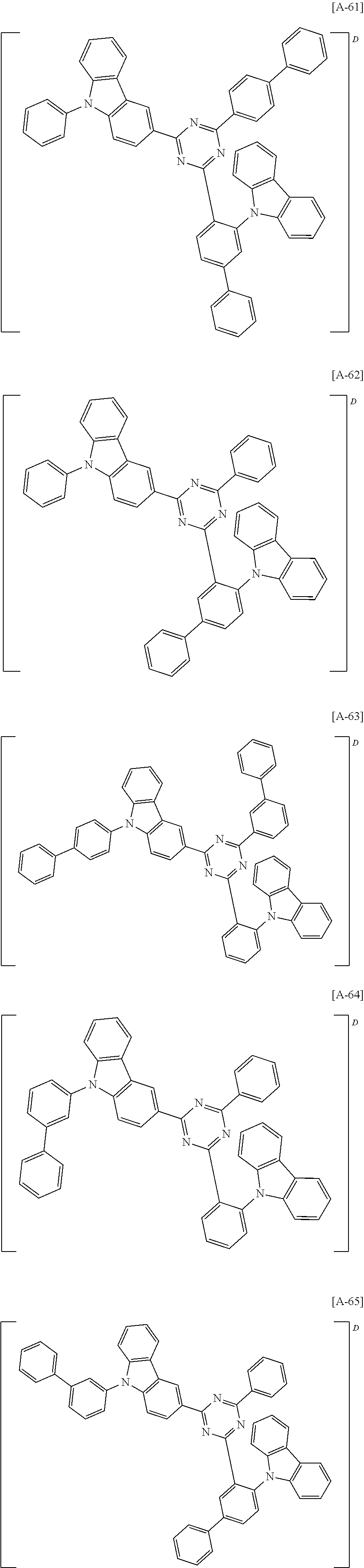

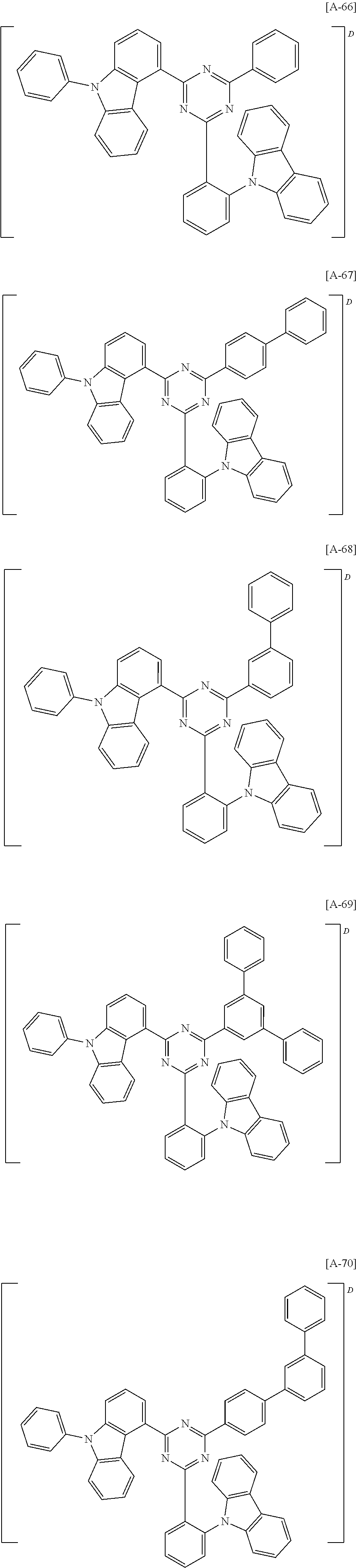

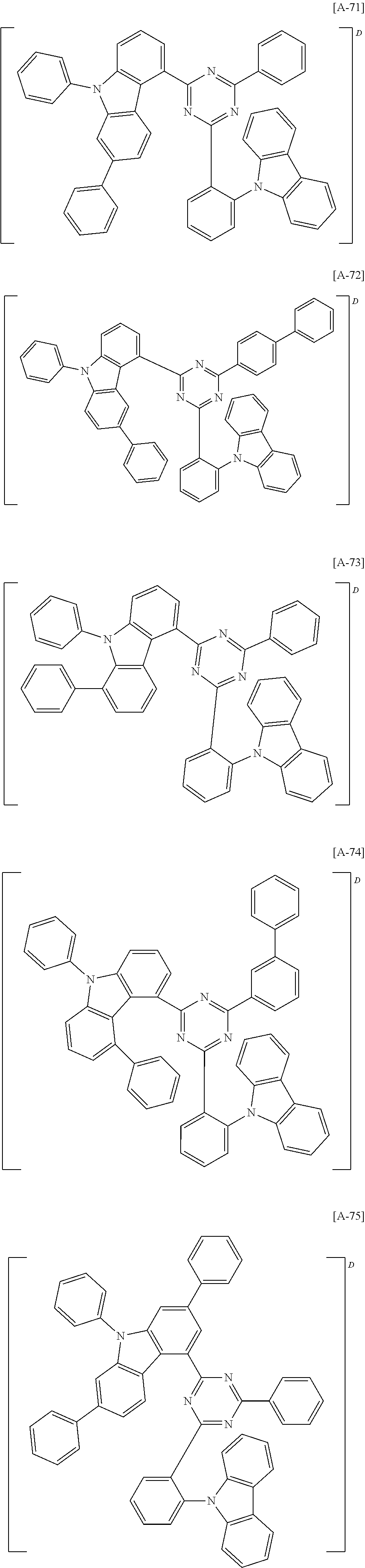

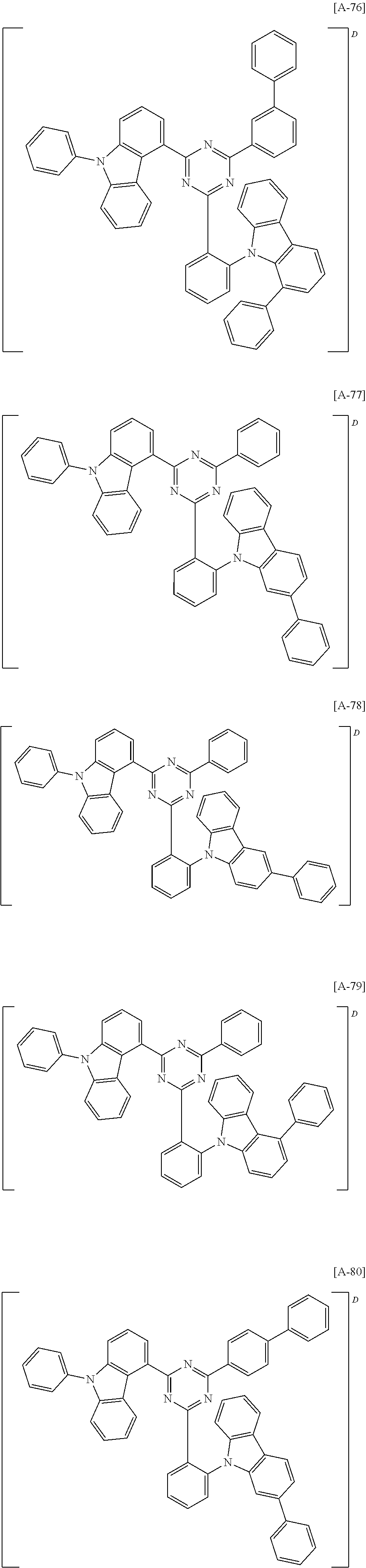

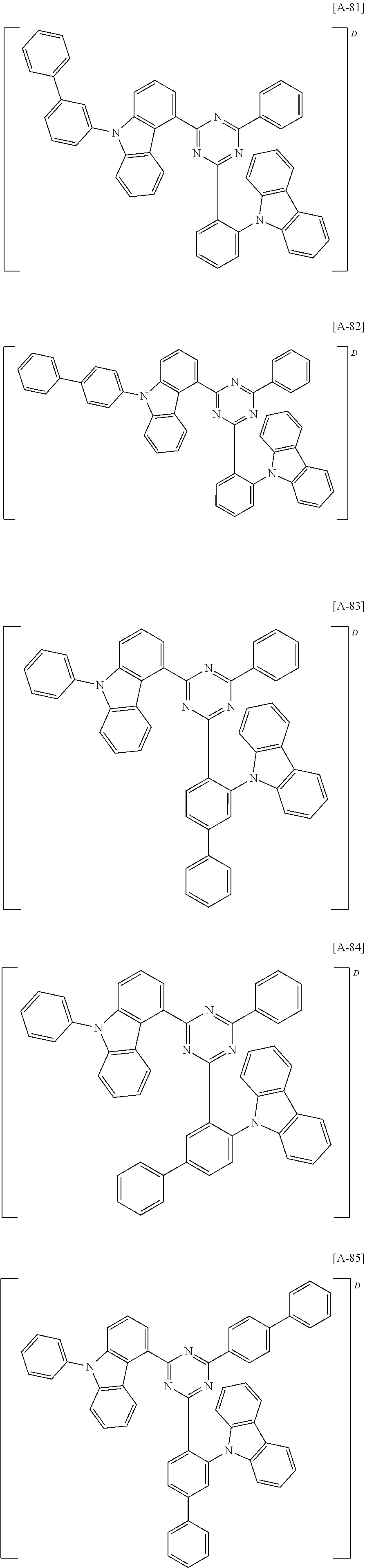

In Compounds A-1 to A-85 of Group 1,
D indicates a structure in which deuterium is substituted, and
the substitution ratio of D may be 10% to 100%, preferably 30% to 100%, and more preferably 50% to 100%.

The most specific structures for Compound A-1 to Compound A-85 are presented below as examples according to the position and substitution ratio of deuterium substitution, which is not to limit the scope of rights to compounds not shown below. The scope of the present invention is determined by the claims.

Examples of Compound A-2 may include Compounds A-86 to A-90, but are not limited thereto.

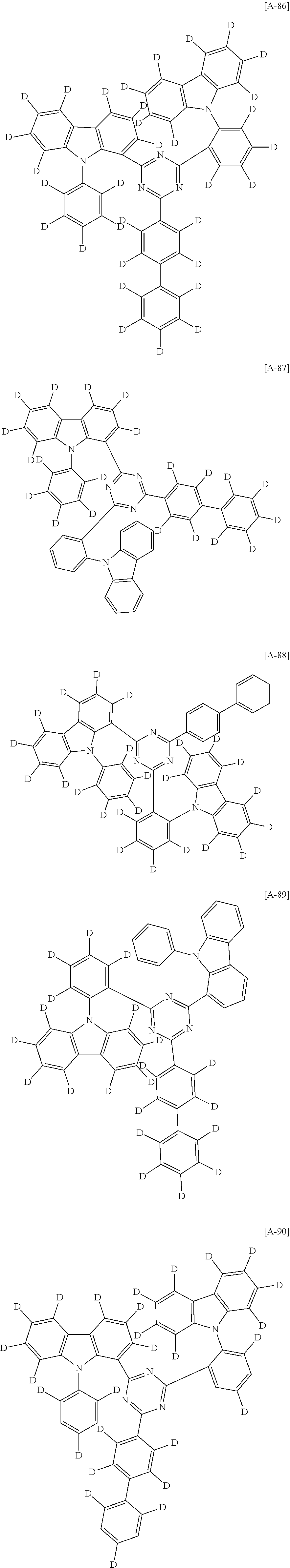

Examples of Compound A-22 may include Compounds A-91 to A-95, but are not limited thereto.

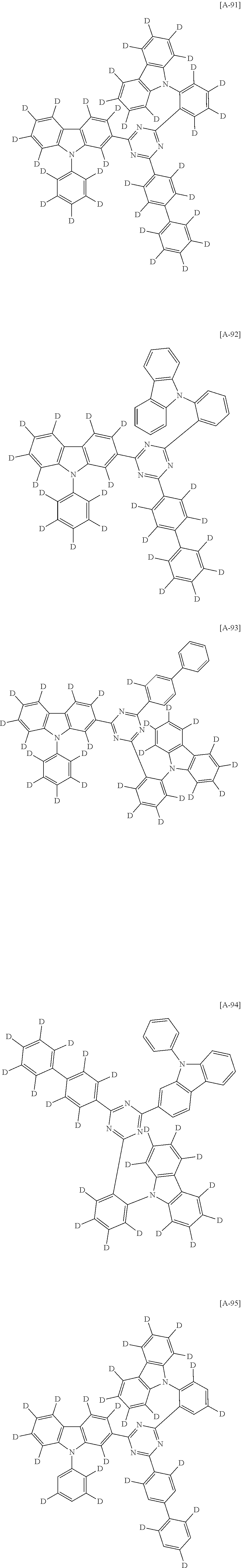

Examples of Compound A-47 may include Compounds A-96 to A-100, but are not limited thereto.

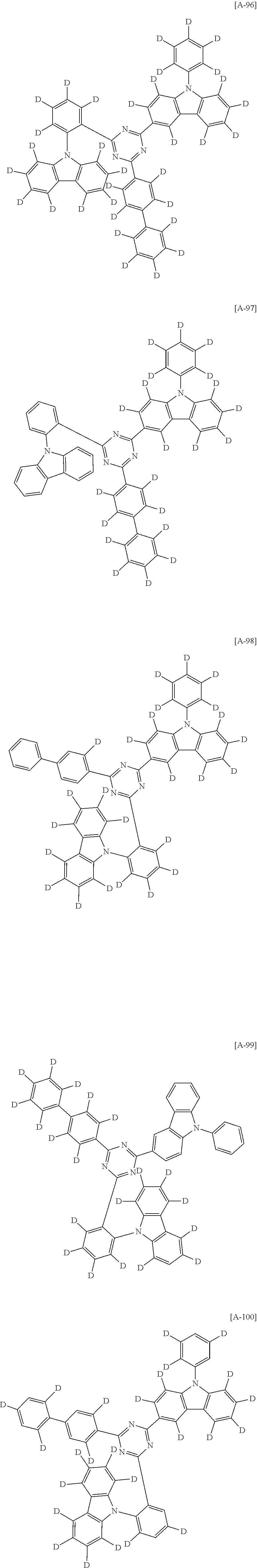

Examples of Compound A-67 may include Compounds A-101 to A-105, but are not limited thereto.

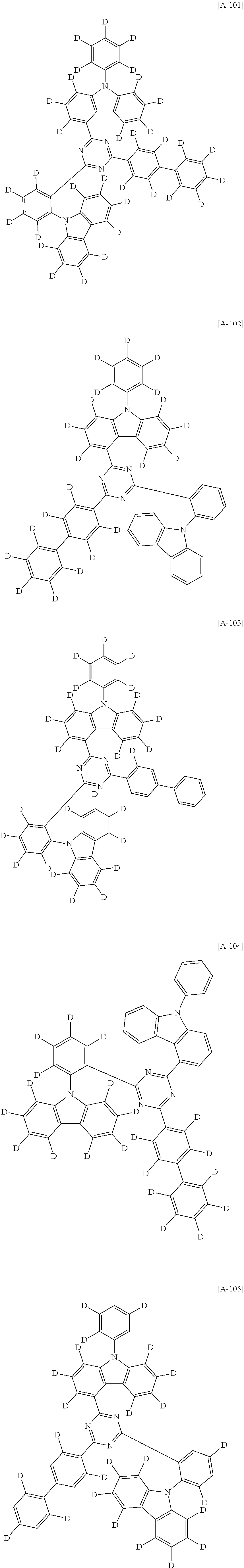

Examples of Compound A-12 may include Compounds A-106 to A-110 but are not limited thereto.

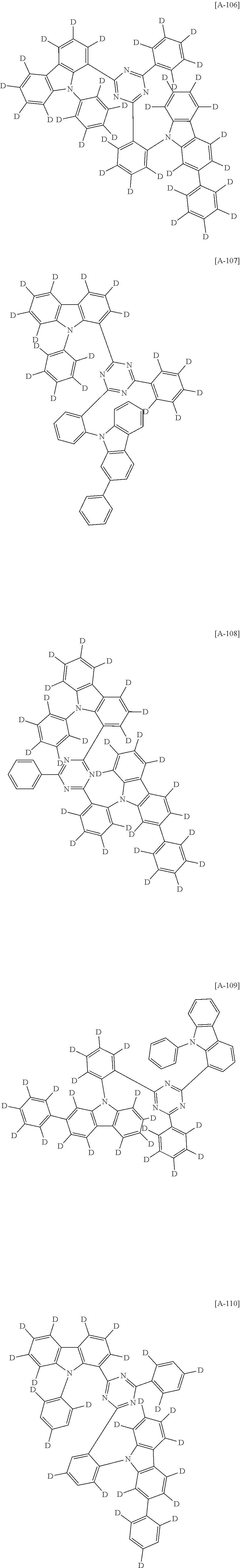

Examples of Compound A-32 may include Compounds A-111 to A-115, but are not limited thereto.

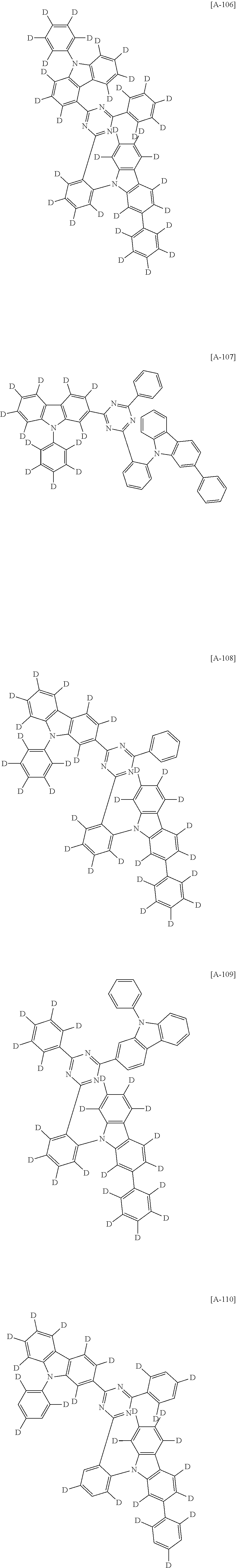

Examples of Compound A-57 may include Compounds A-116 to A-120, but are not limited thereto.

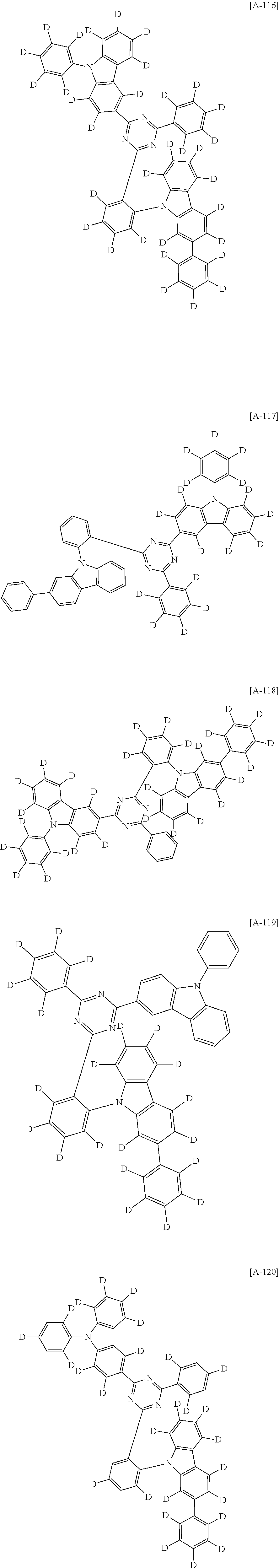

Examples of Compound A-77 may include Compounds A-121 to A-125, but are not limited thereto.

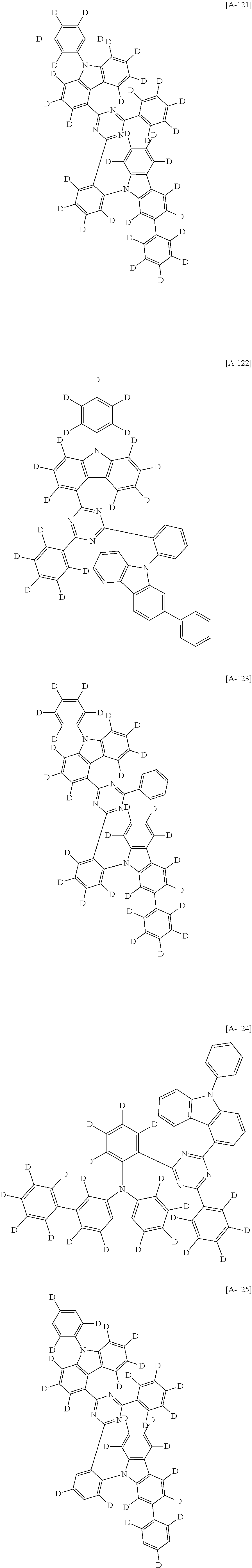

In addition to the aforementioned compound for the organic optoelectronic device, one or more additional compounds may be included.

For example, a dopant may be further included.

The dopant may be, for example, a phosphorescent dopant, for example, a red, green or blue phosphorescent dopant, and may be, for example, a red or green phosphorescent dopant.

The dopant is a material mixed with the compound for the organic optoelectronic device in a small amount to cause light emission, and may be generally a material such as a metal complex that emits light by multiple excitation into a triplet or more. The dopant may be, for example, an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example, a compound represented by Chemical Formula Z, but is not limited thereto.

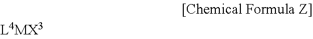

In Chemical Formula Z, M is a metal, and L⁴ and X³ are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof and the L⁴ and X³ may be, for example, a bidentate ligand.

Examples of ligands represented by L⁴ and X³ may be selected from the Chemical Formulas listed in Group A, but are not limited thereto.

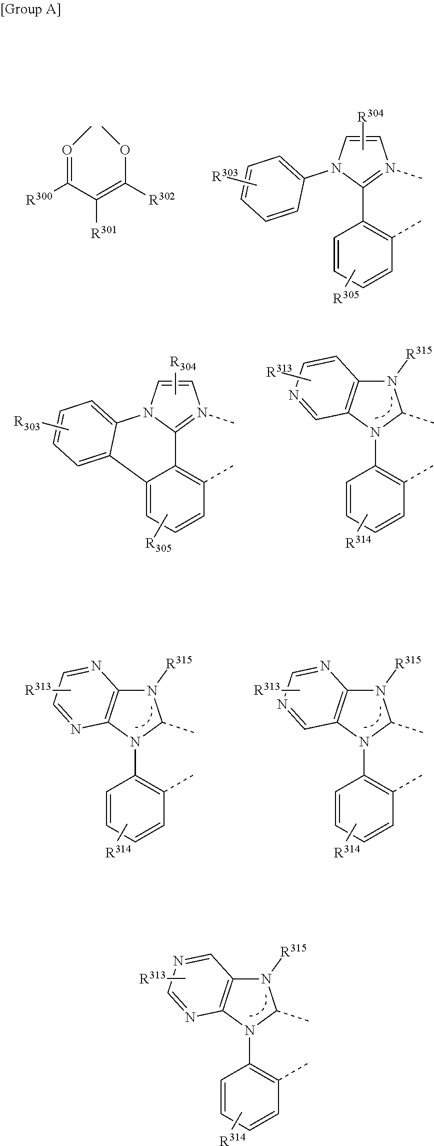

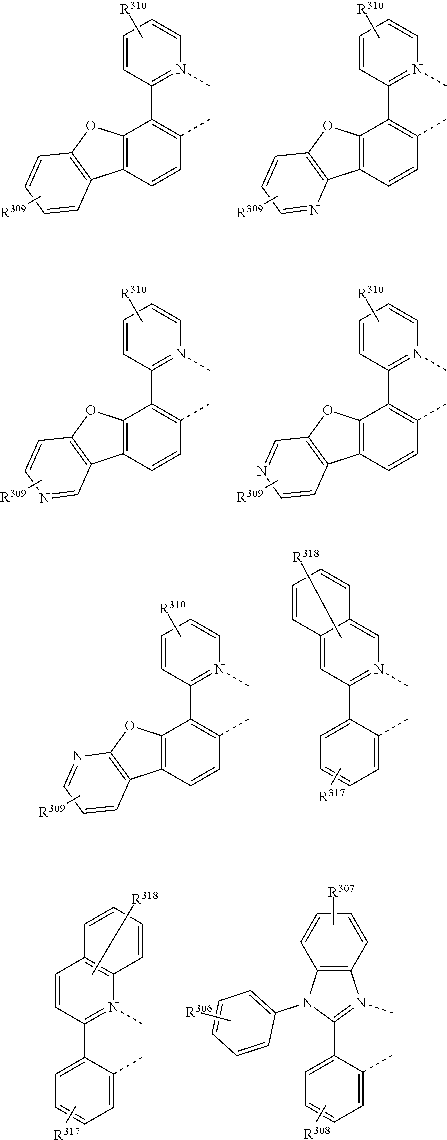

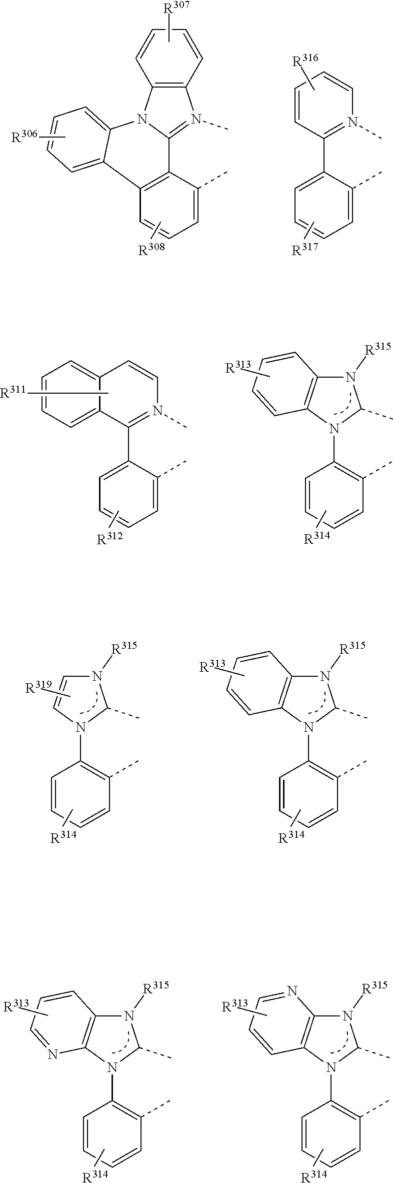

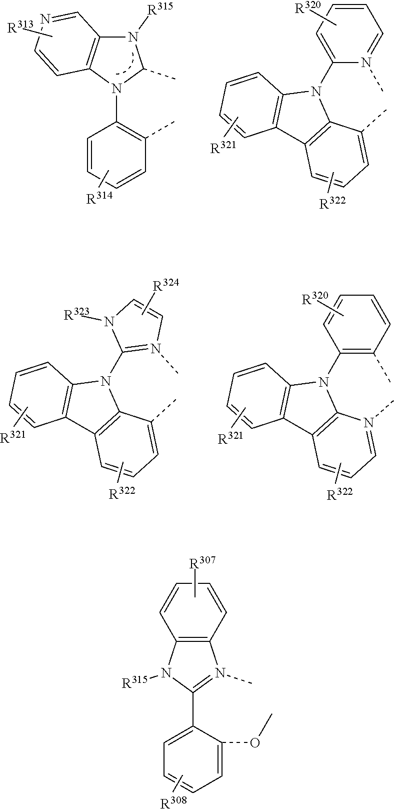

In Group A,
R³⁰⁰ to R³⁰² are each independently hydrogen, deuterium, a C1 to C30 alkyl group substituted or unsubstituted with halogen, a C6 to C30 aryl group substituted or unsubstituted with C1 to C30 alkyl or a halogen, and
R³⁰³ to R³²⁴ are each independently hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

The dopant according to an embodiment may be an iridium complex, and may be represented, for example, by Chemical Formula 5-1 or Chemical Formula 5-2.

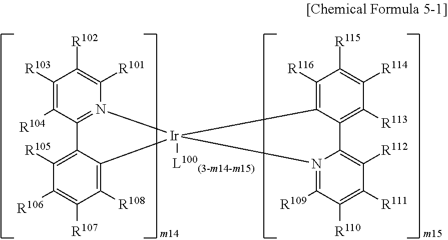

In Chemical Formula 5-1,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a substituted or unsubstituted C1 to C6 alkyl group,
at least one of R¹⁰¹ to R¹¹⁶ is a functional group represented by Chemical Formula V-1,
L¹⁰⁰ is a bidentate ligand of a monovalent anion, and is a ligand that coordinates to iridium through a lone pair of carbons or heteroatoms, and
m14 and m15 are each independently any one of integers of 0 to 3, and m14+m15 is any one of integers of 1 to 3,

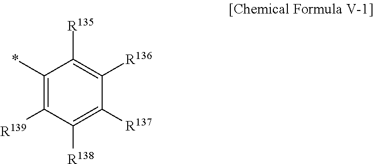

wherein, in Chemical Formula V-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
* refers to a portion linked to a carbon atom.

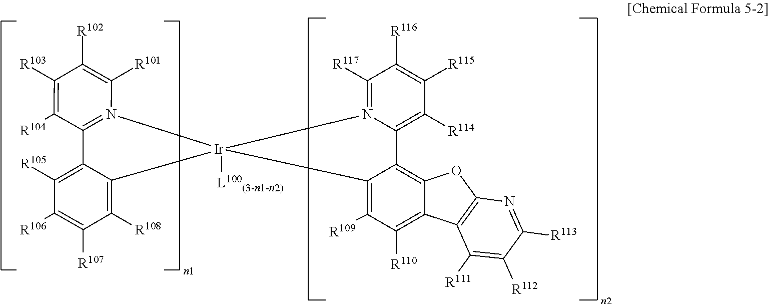

wherein, in Chemical Formula 5-2,
R¹⁰¹ to R¹¹⁷ are each independently hydrogen, deuterium, a substituted or unsubstituted C1 to C10 alkyl group, a substituted or unsubstituted C6 to C20 aryl group, or —SiR¹³³R¹³⁴R¹³⁵,
R¹³³ to R¹³⁵ are each independently a substituted or unsubstituted C1 to C6 alkyl group,
L¹⁰⁰ is a bidentate ligand of a monovalent anion, and is a ligand that coordinates to iridium through a lone pair of carbons or heteroatoms, and
n1 and n2 are each independently any one of integers of 0 to 3, and n1+n2 is any one of integers of 1 to 3.

In another embodiment, the dopant may be a platinum complex, for example, represented by Chemical Formula Z-1.

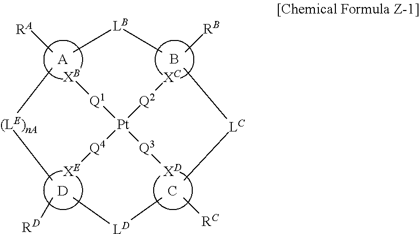

In Chemical Formula Z-1, rings A, B, C, and D are each independently a 5-membered or 6-membered carbocyclic or heterocyclic ring;
R^{A}, R^{B}, R^{C}, and R^{D} are each independently mono-, di-, tri-, or tetra-substitution, or unsubstitution;
L^{B}, L^{C}, and L^{D} are each independently a direct bond, BR, NR, PR, O, S, Se, C═O, S═O, SO₂, CRR′, SiRR′, GeRR′, or a combination thereof,
when nA is 1, L^{E} may be a direct bond, BR, NR, PR, O, S, Se, C═O, S═O, SO₂, CRR′, SiRR′, GeRR′, or a combination thereof, and when nA is 0, L^{E} does not exist;
R^{A}, R^{B}, R^{C}, R^{D}, R, and R′ are each independently hydrogen, deuterium, a halogen, alkyl group, a cycloalkyl group, a heteroalkyl group, arylalkyl group, an alkoxy group, aryloxy group, an amino group, a silyl group, an alkenyl group, a cycloalkenyl group, a heteroalkenyl group, an alkynyl group, aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}, R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The platinum complex may be represented, for example, by Chemical Formula 6-1 or Chemical Formula 6-2.

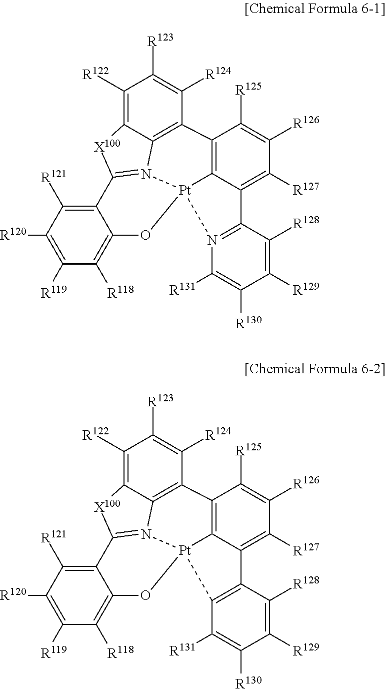

In Chemical Formula 6-1 and Chemical Formula 6-2,
X¹⁰⁰ is selected from O, S, and NR¹³²,
R¹¹⁸ to R¹³² are each independently hydrogen, deuterium, a substituted or unsubstituted C1 to C10 alkyl group, a substituted or unsubstituted C6 to C20 aryl group, or —SiR¹³³R¹³⁴R¹³⁵,
R¹³³ to R¹³⁵ are each independently a substituted or unsubstituted C1 to C6 alkyl group,
at least one of R¹¹⁸ to R¹³² is —SiR¹³³R¹³⁴R¹³⁵ or a tert-butyl group, and
R¹³³ to R¹³⁵ are each independently a substituted or unsubstituted C1 to C6 alkyl group.

Hereinafter, an organic optoelectronic device including the aforementioned compound for the organic optoelectronic device is described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

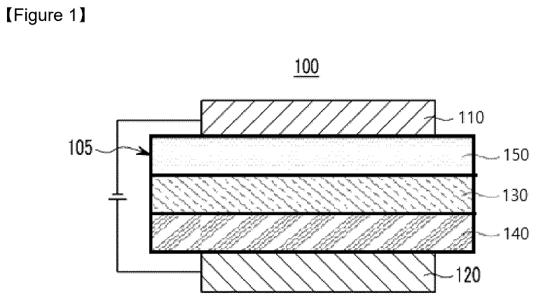

FIG. 1 is a cross-sectional view showing an organic light emitting diode according to an embodiment.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer.

The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, LiF/Al, and BaF₂/Ca, but is not limited thereto.

The organic layer 105 may include the aforementioned compound for the organic optoelectronic device.

The compound for the organic optoelectronic device further including a dopant may be, for example, a green light emitting composition.

The light emitting layer 130 may include, for example, the aforementioned compound for the organic optoelectronic device as a phosphorescent host.

The light emitting layer 130 may include, for example, the aforementioned compound for the organic optoelectronic device as a phosphorescent host.

The organic layer may further include a charge transport region in addition to the light emitting layer.

The charge transport region may be, for example, a hole transport region 140.

The hole transport region 140 can further increase hole injection and/or hole mobility and block electrons between the anode 120 and the light emitting layer 130.

Specifically, the hole transport region 140 may include a hole transport layer between the anode 120 and the light emitting layer 130, and a hole transport auxiliary layer between the light emitting layer 130 and the hole transport layer, and at least one of the compounds listed in Group B may be included in at least one layer of the hole transport layer and the hole transport auxiliary layer.

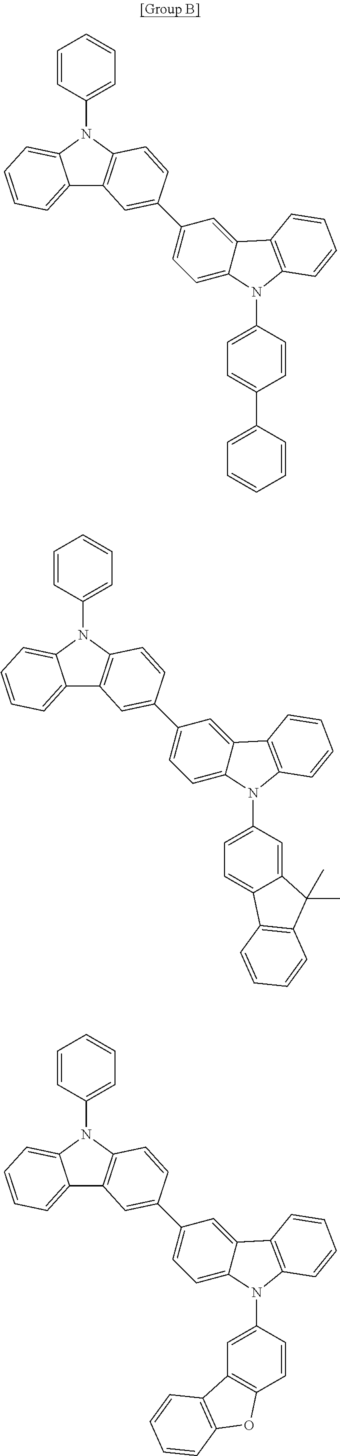 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

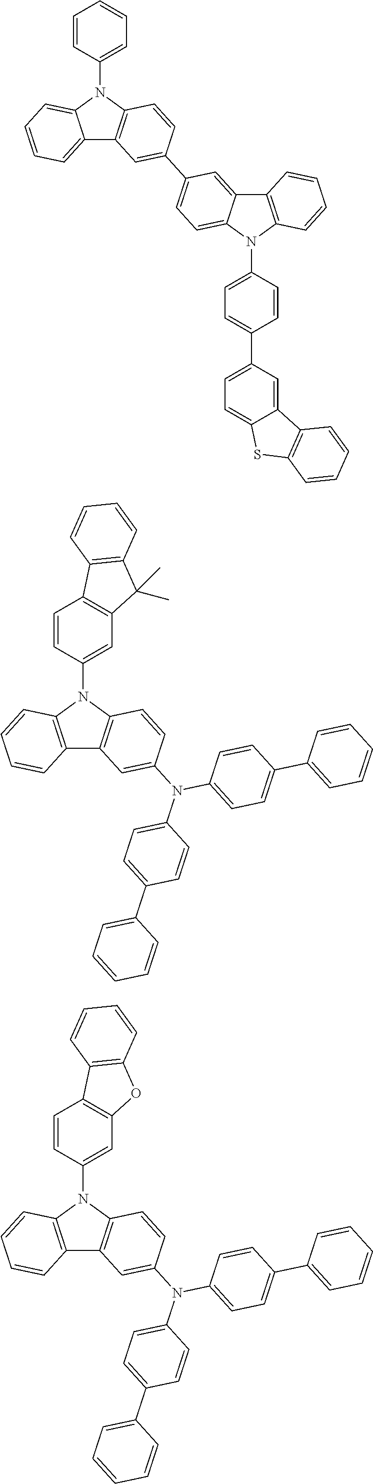

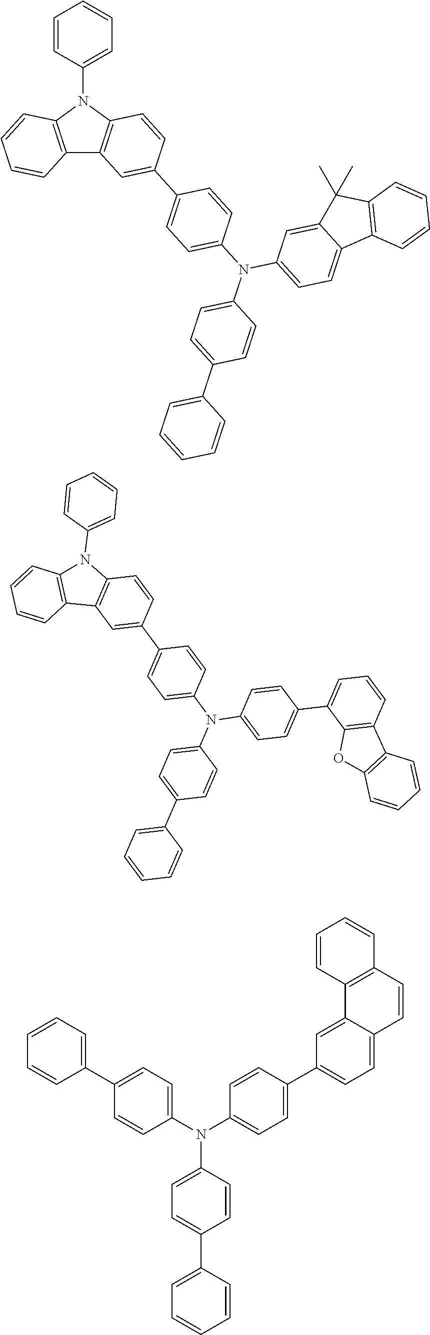 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1]

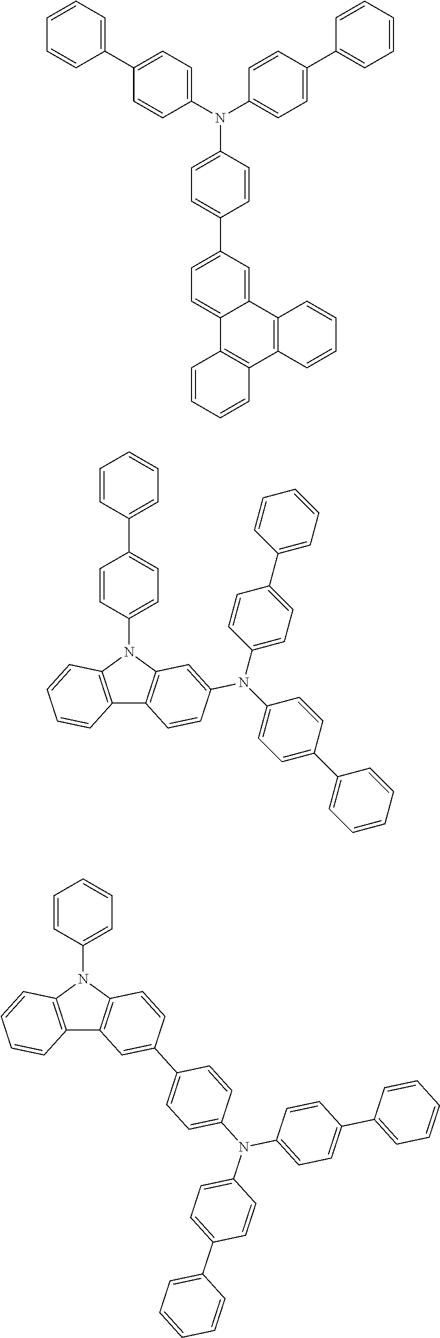 [SMILES: c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

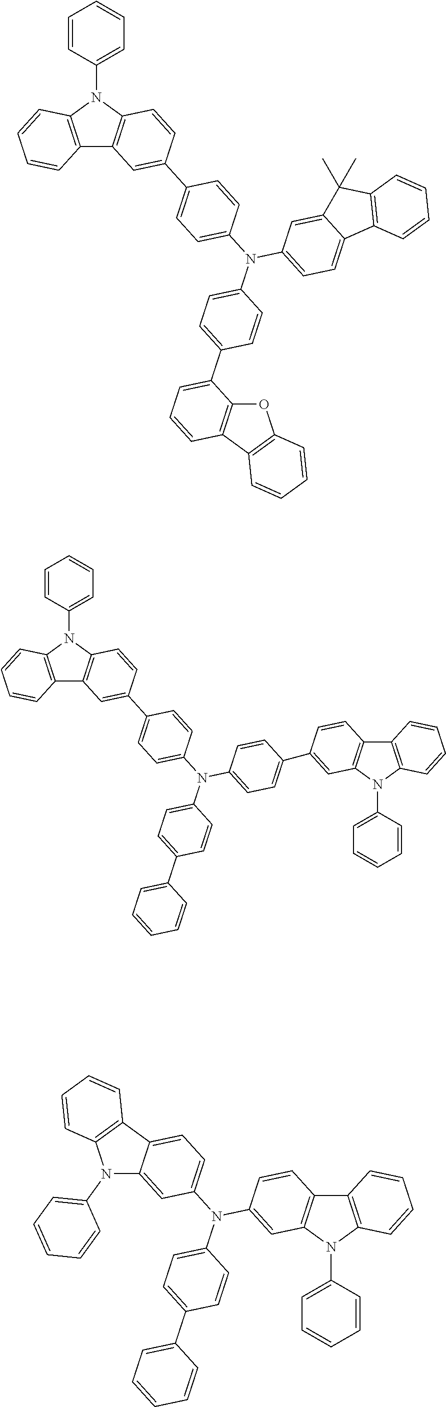 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1]

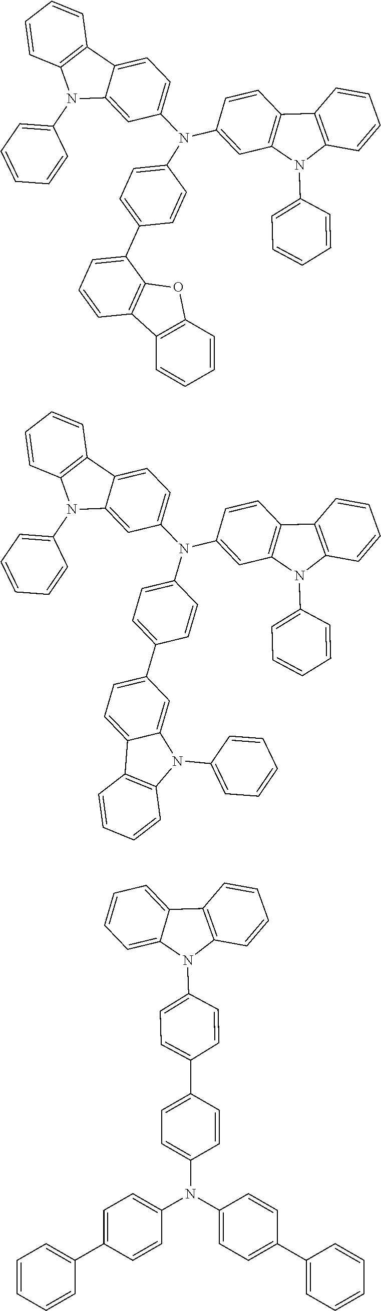 [SMILES: c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1 ; c1ccc(-c2ccc(N(c3ccc(-c4ccccc4)cc3)c3ccc(-c4ccc(-n5c6ccccc6c6ccccc65)cc4)cc3)cc2)cc1]

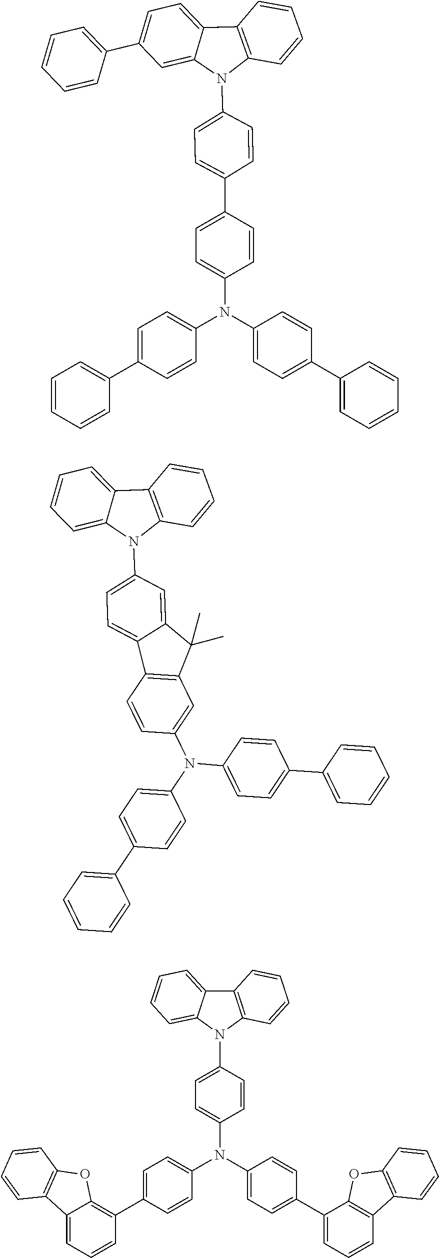

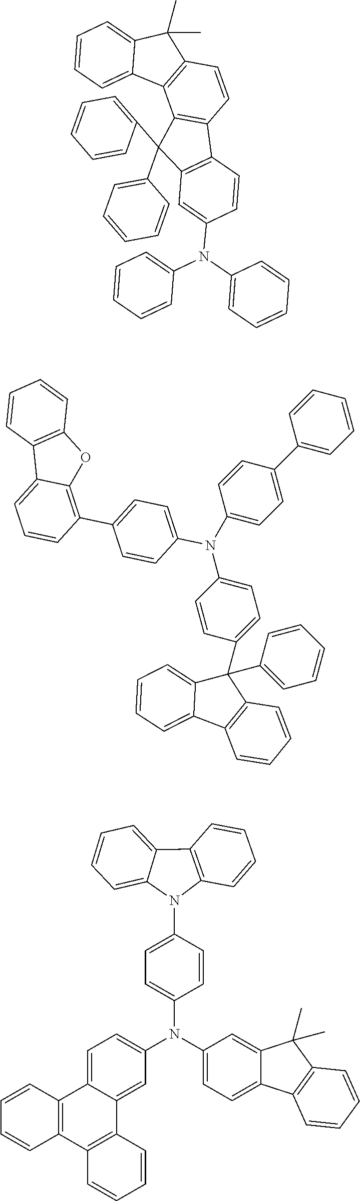 [SMILES: CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; CC1(C)c2ccccc2-c2ccc(N(c3ccc(-n4c5ccccc5c5ccccc54)cc3)c3ccc4c5ccccc5c5ccccc5c4c3)cc21]

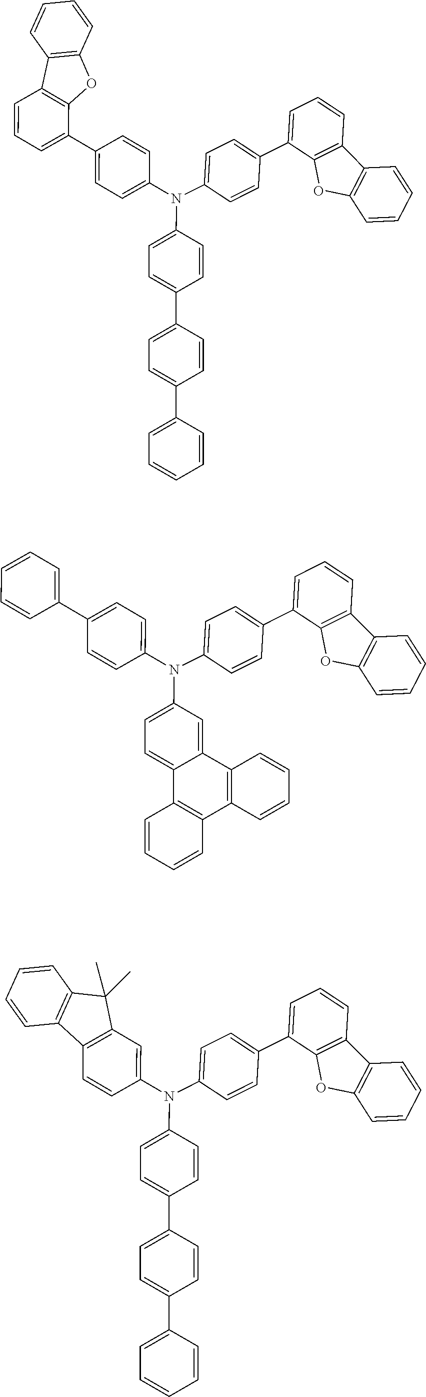 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21]

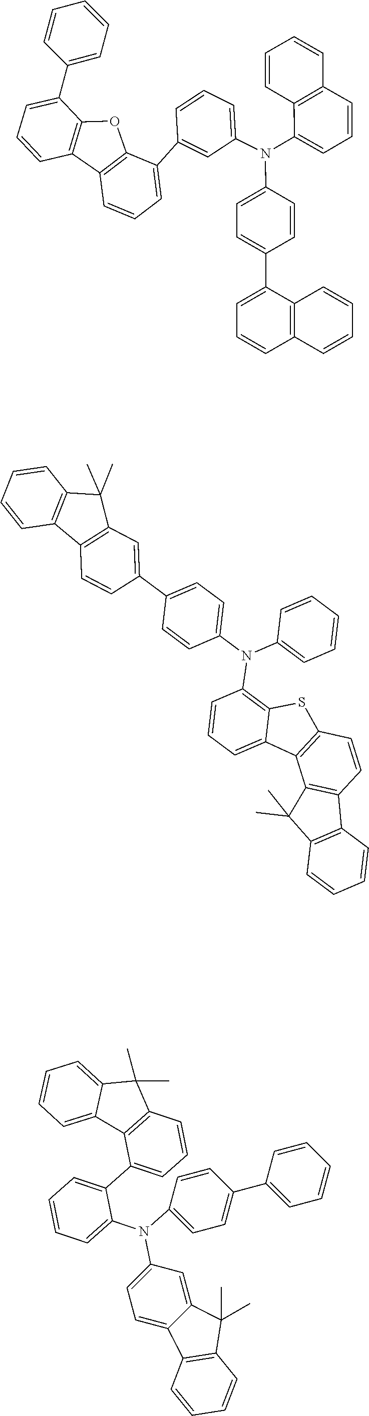 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

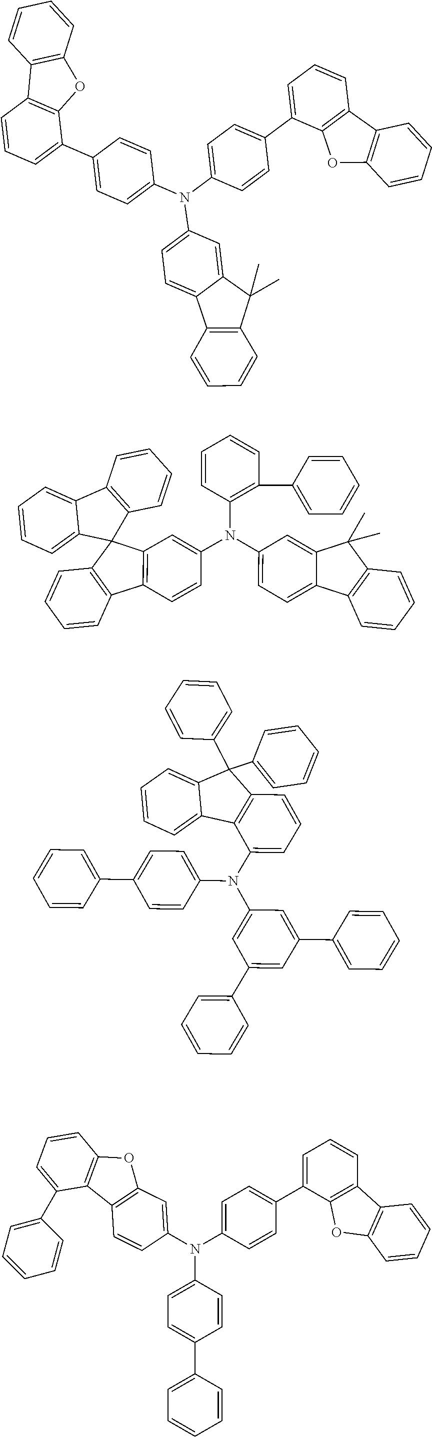 [SMILES: CC1(C)c2ccccc2-c2ccc(N(c3ccc(-c4cccc5c4oc4ccccc45)cc3)c3ccc(-c4cccc5c4oc4ccccc45)cc3)cc21 ; CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1]

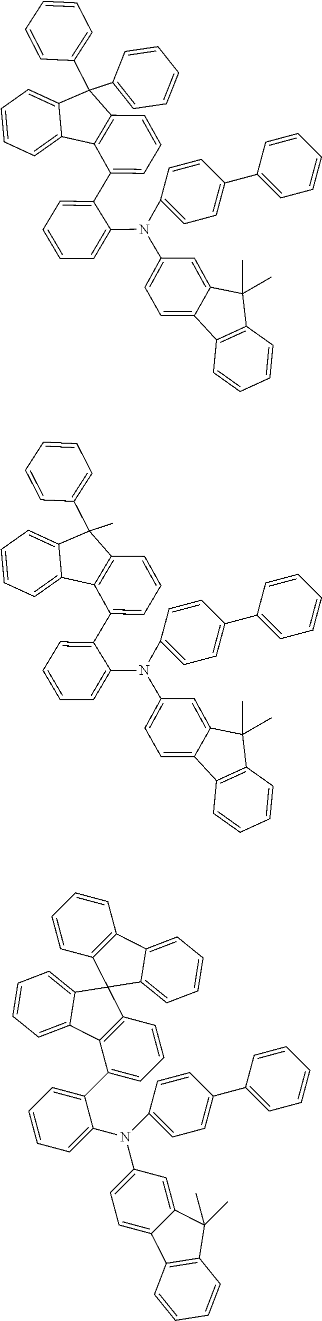 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

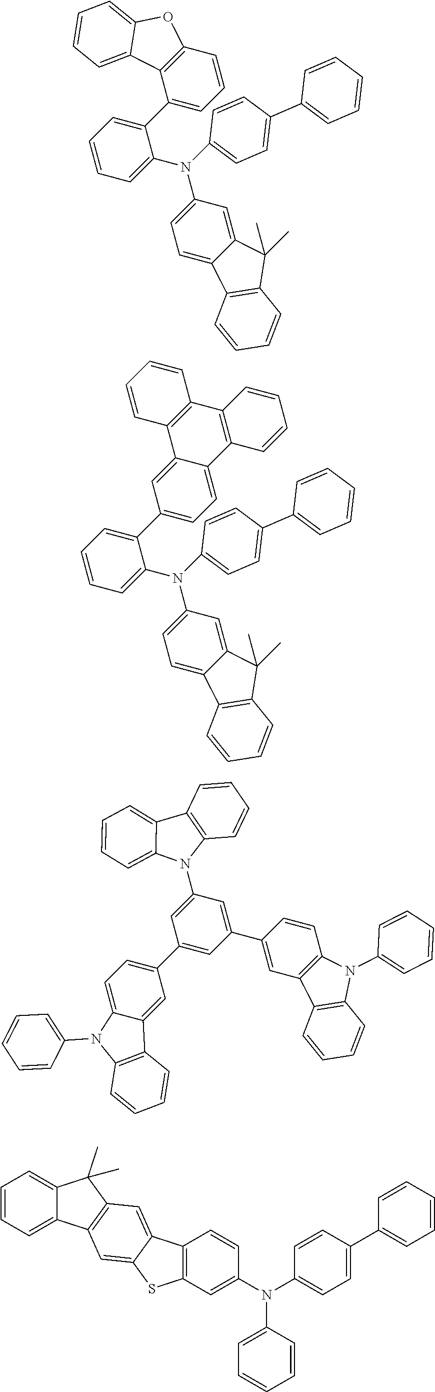

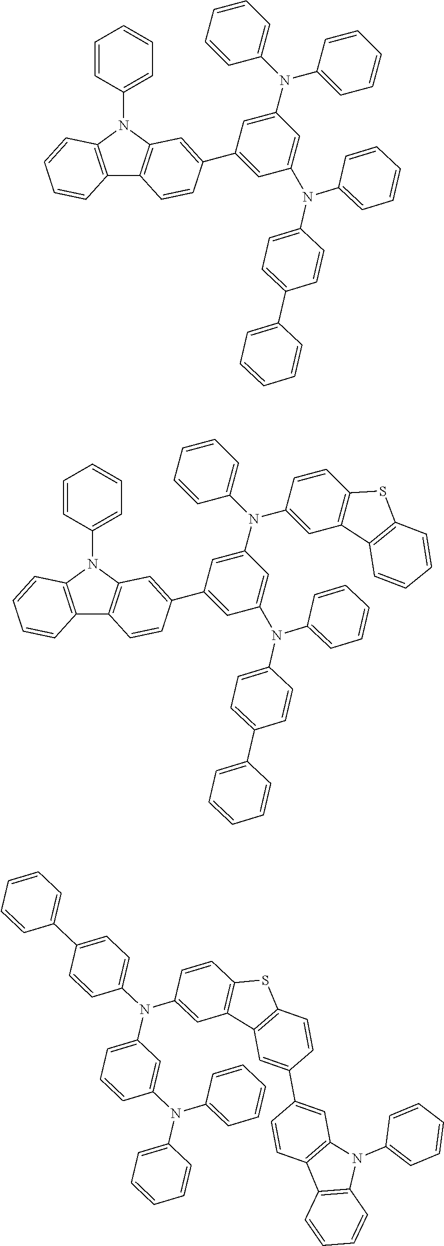 [SMILES: c1ccc(-c2ccc(N(c3ccccc3)c3cc(-c4ccc5c6ccccc6n(-c6ccccc6)c5c4)cc(N(c4ccccc4)c4ccccc4)c3)cc2)cc1 ; c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1]

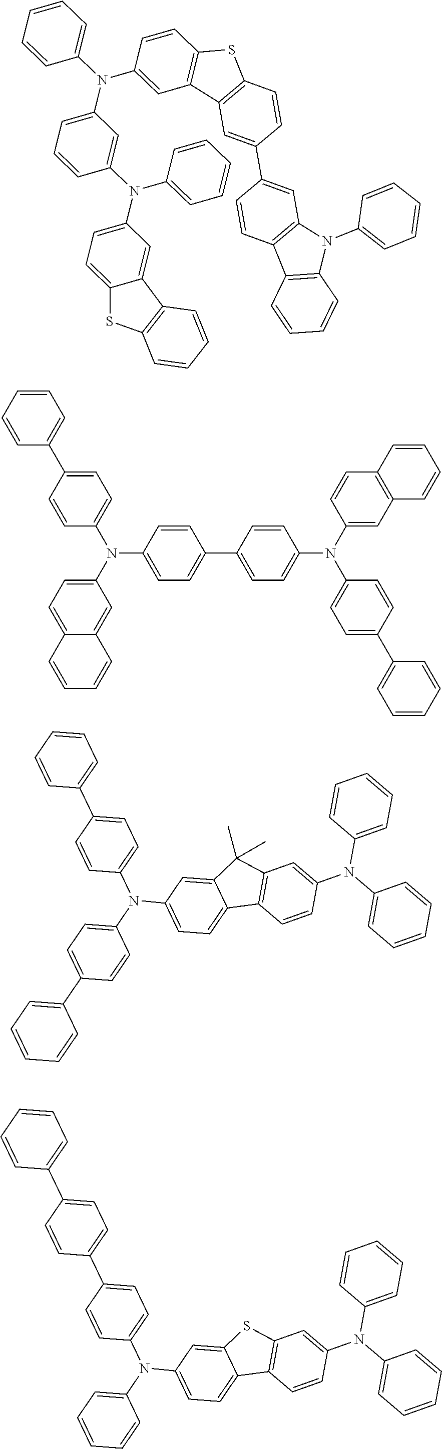 [SMILES: c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21 ; c1ccc(-c2ccc(-c3ccc(N(c4ccccc4)c4ccc5c(c4)sc4cc(N(c6ccccc6)c6ccccc6)ccc45)cc3)cc2)cc1]

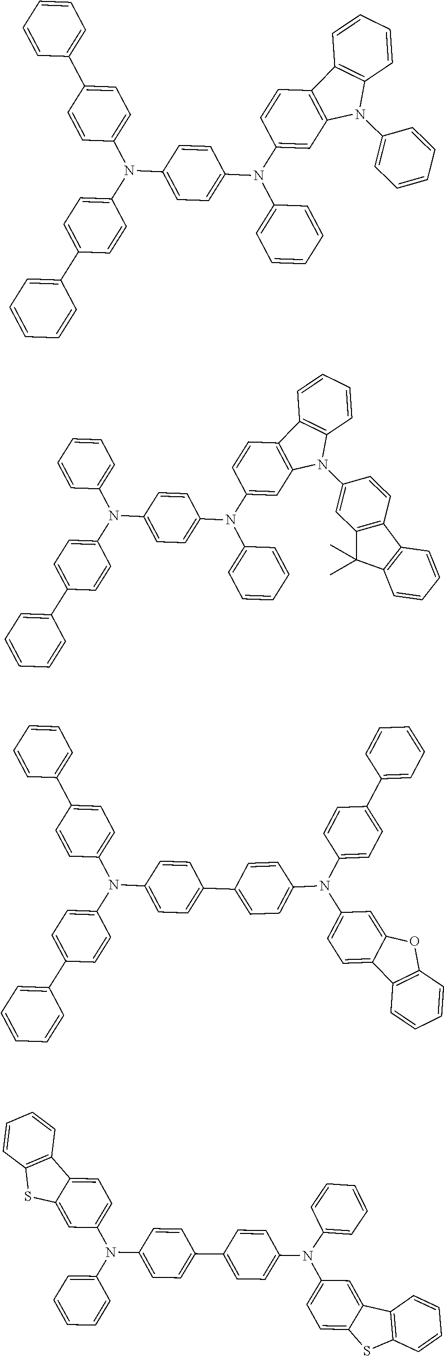 [SMILES: c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21 ; c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1]

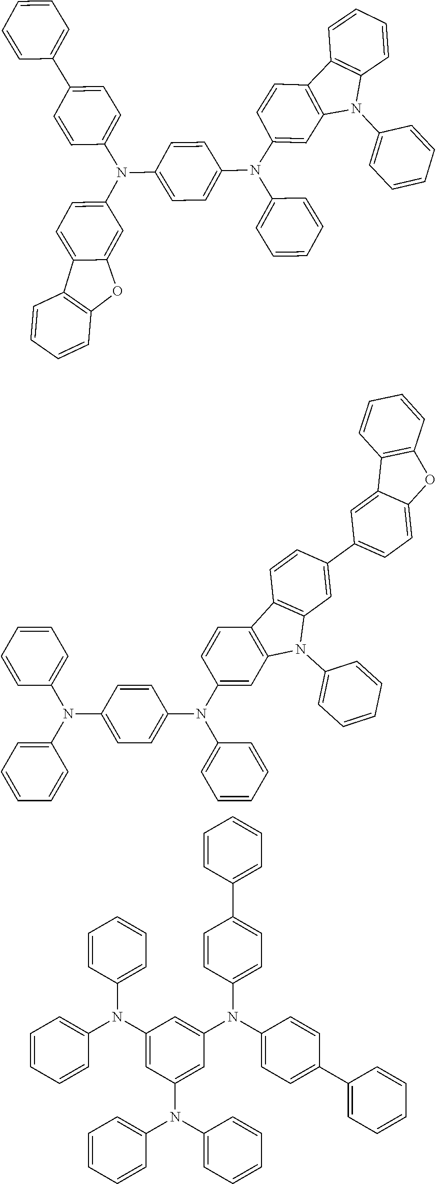

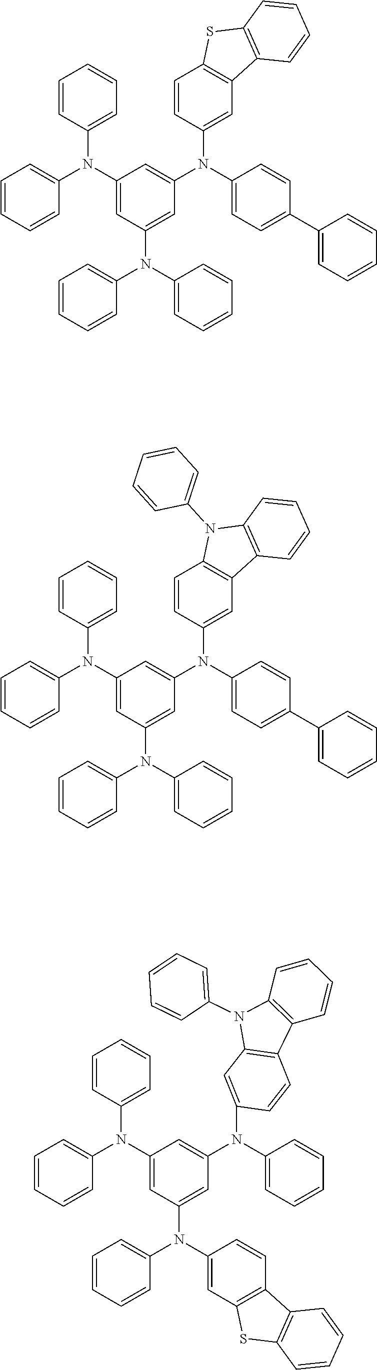 [SMILES: c1ccc(-c2ccc(N(c3cc(N(c4ccccc4)c4ccccc4)cc(N(c4ccccc4)c4ccccc4)c3)c3ccc4sc5ccccc5c4c3)cc2)cc1 ; c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1]

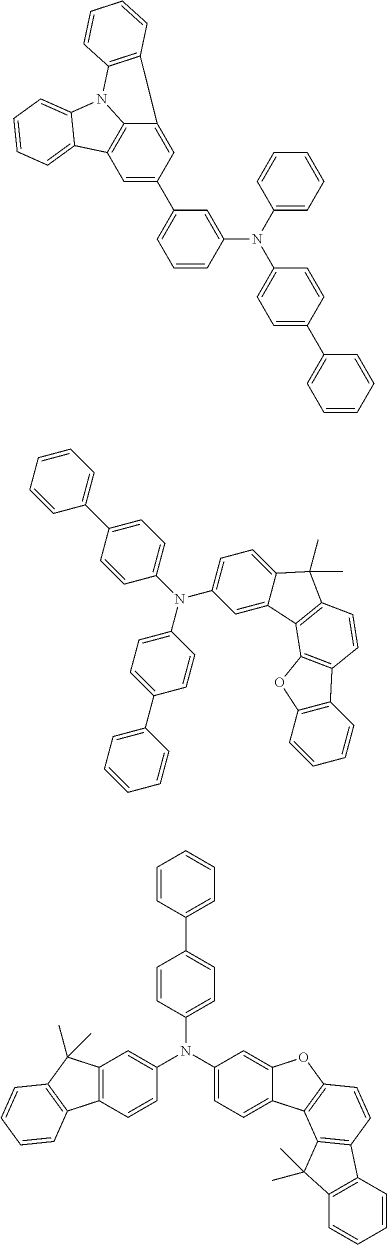 [SMILES: c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21 ; CC1(C)c2ccccc2-c2ccc(N(c3ccc(-c4ccccc4)cc3)c3ccc4c(c3)oc3ccc5c(c34)C(C)(C)c3ccccc3-5)cc21]

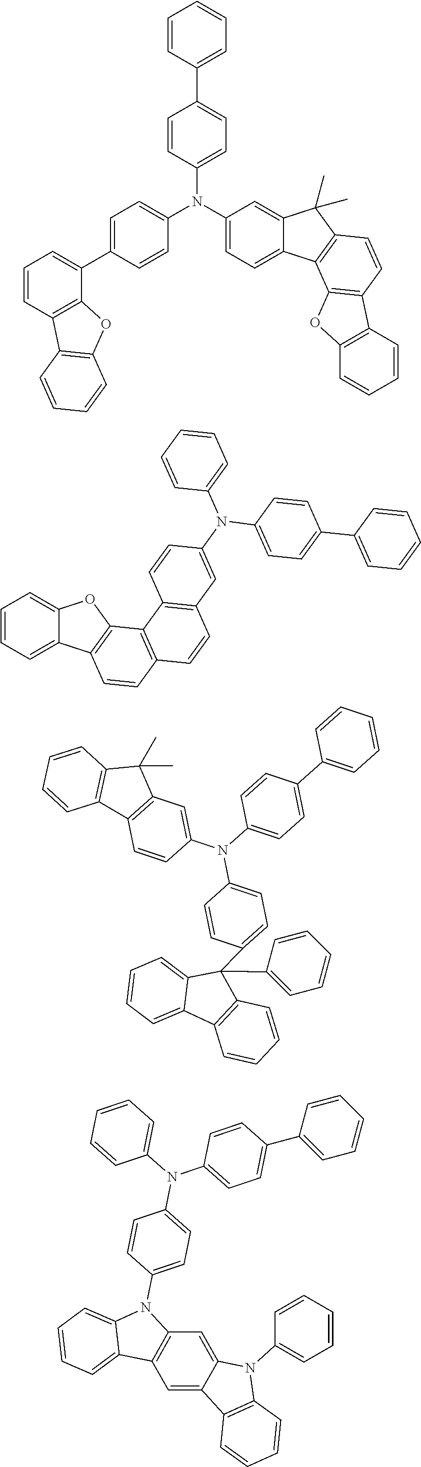 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1]

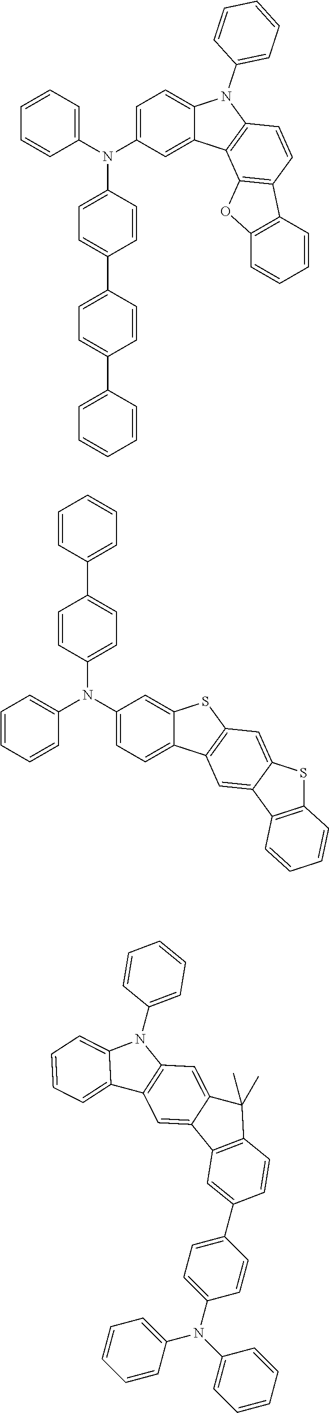

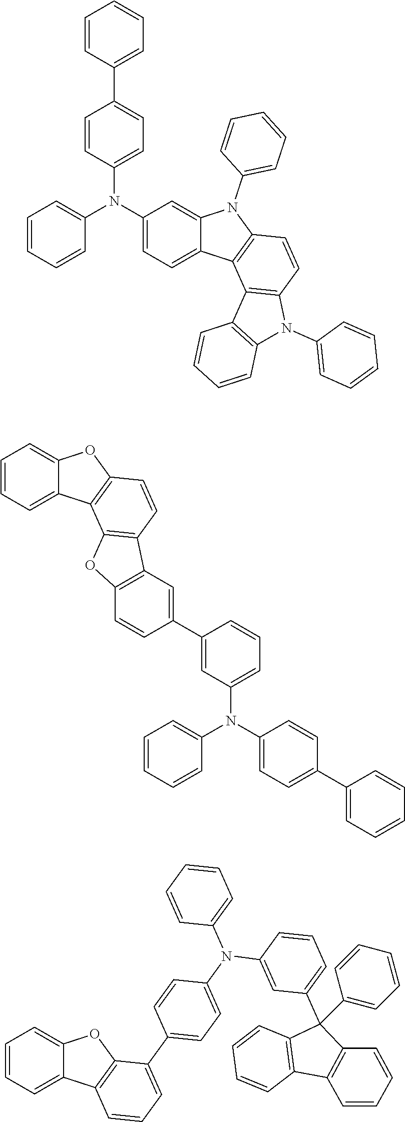 [SMILES: c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1]

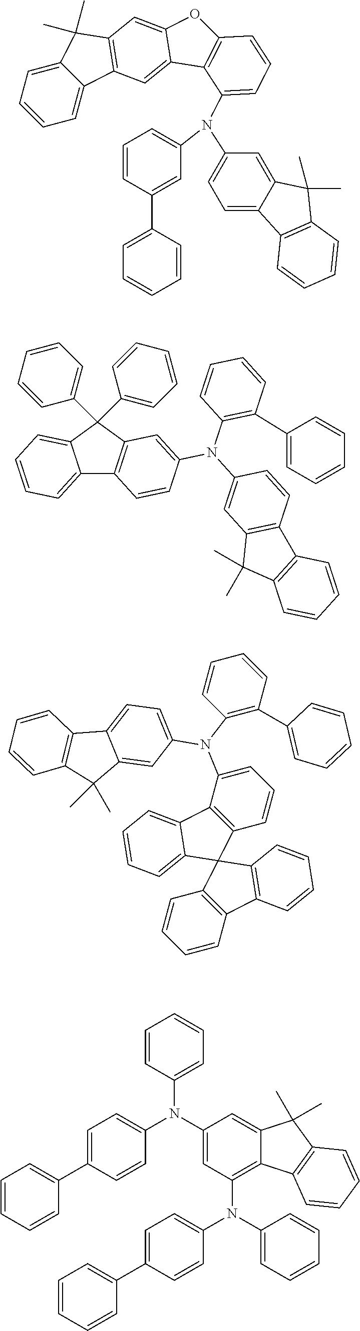 [SMILES: CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2c(N(c3ccccc3)c3ccc(-c4ccccc4)cc3)cc(N(c3ccccc3)c3ccc(-c4ccccc4)cc3)cc21]

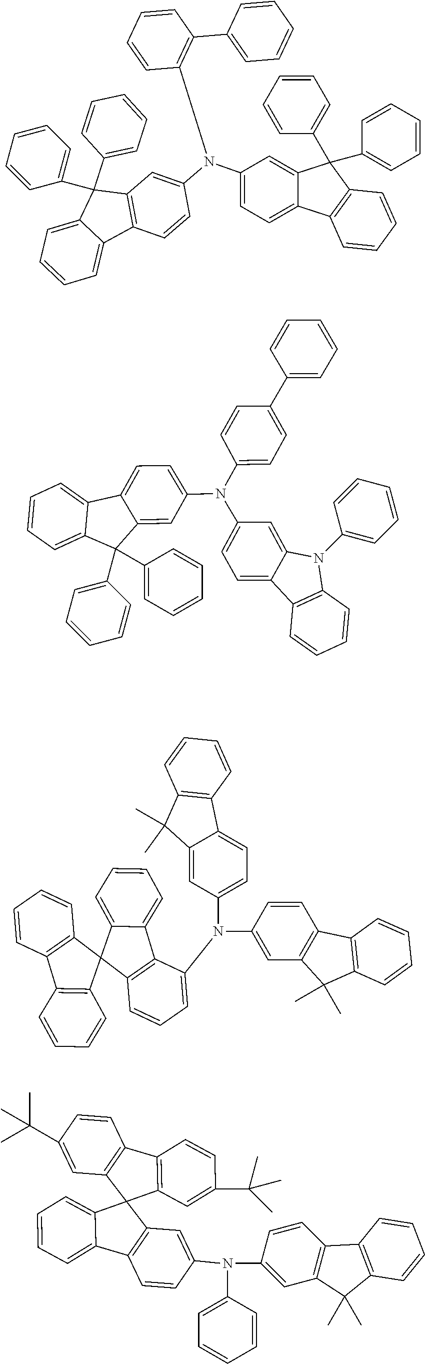 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2]

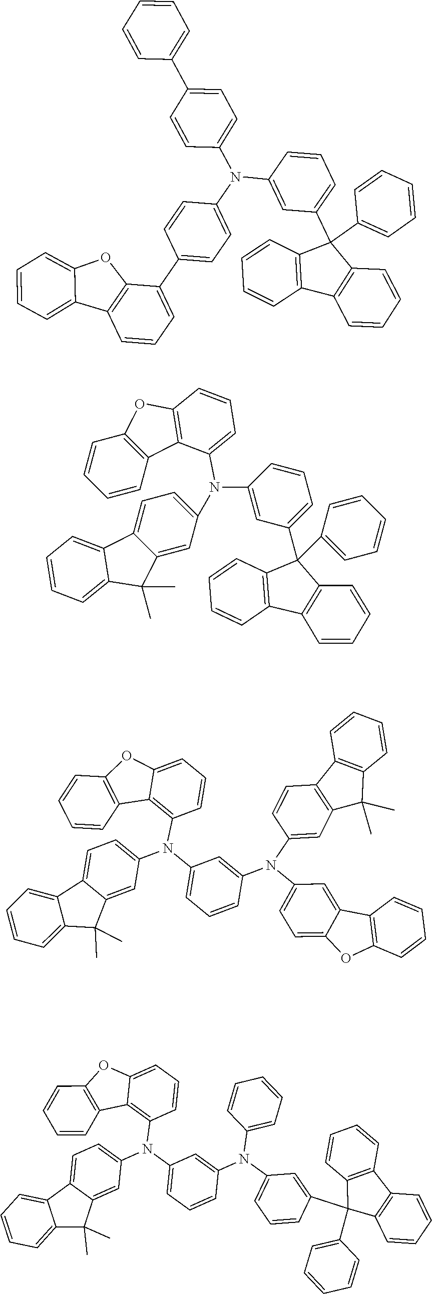 [SMILES: c1ccc(-c2ccc(N(c3ccc(-c4cccc5c4oc4ccccc45)cc3)c3cccc(C4(c5ccccc5)c5ccccc5-c5ccccc54)c3)cc2)cc1 ; CC1(C)c2ccccc2-c2ccc(N(c3cccc(C4(c5ccccc5)c5ccccc5-c5ccccc54)c3)c3cccc4oc5ccccc5c34)cc21 ; CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21 ; CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21]

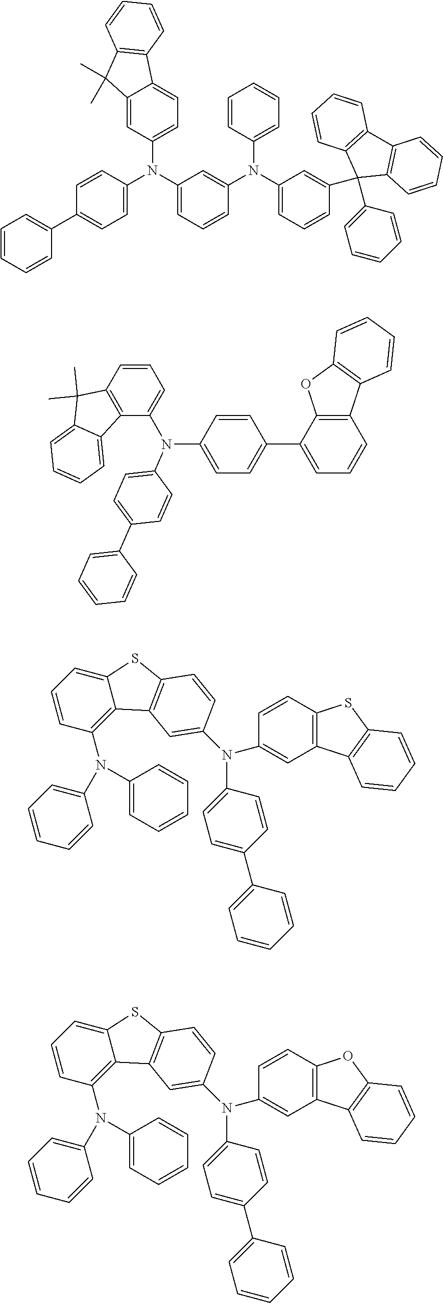 [SMILES: CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21 ; c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1]

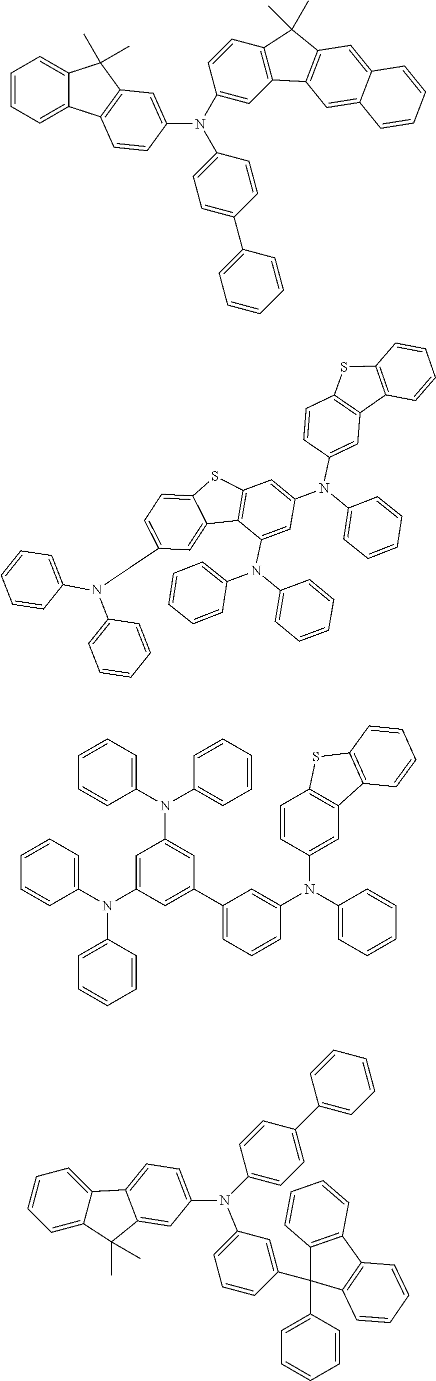 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

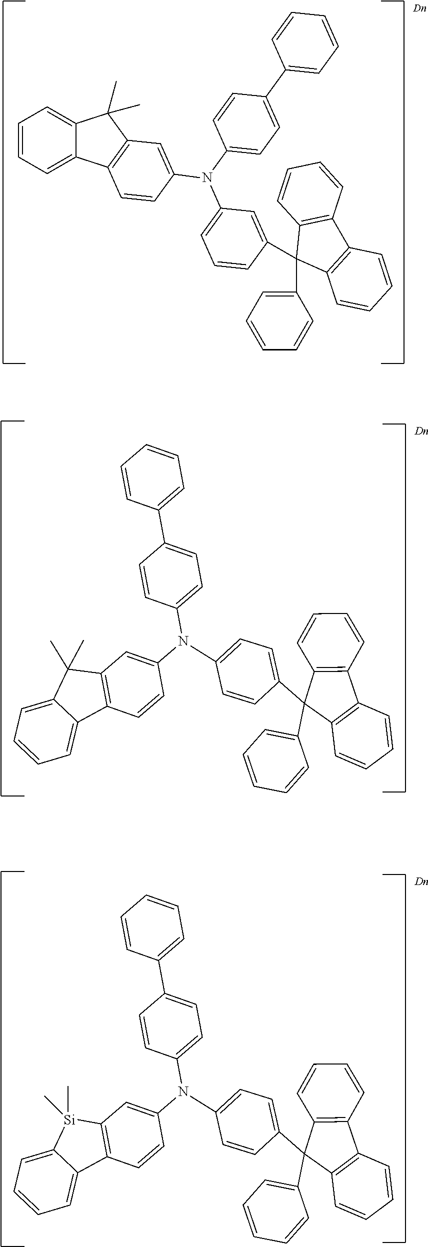

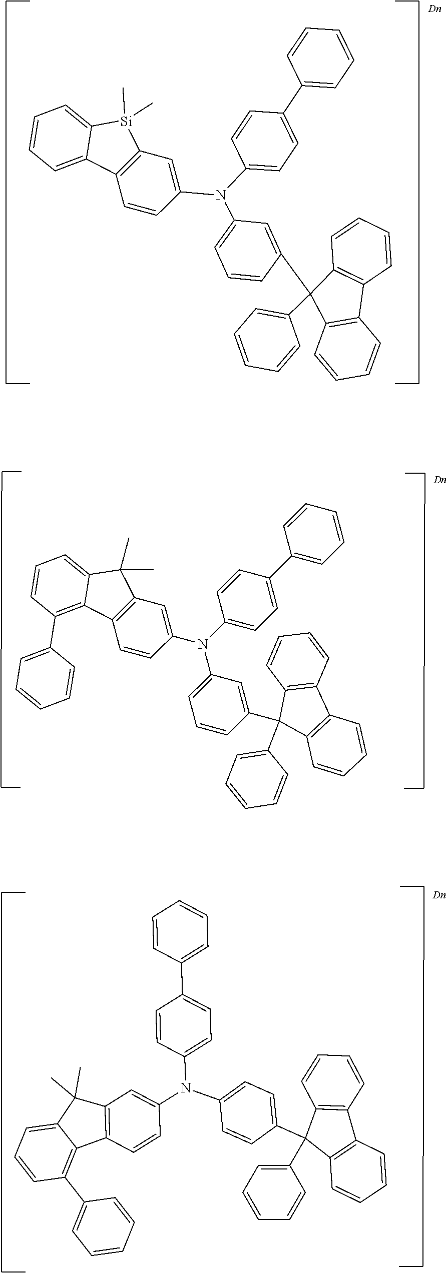

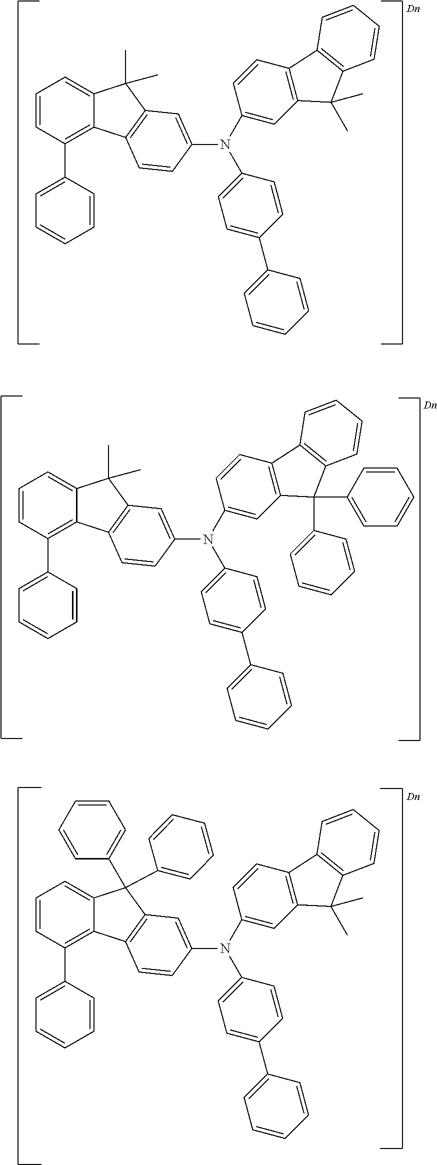

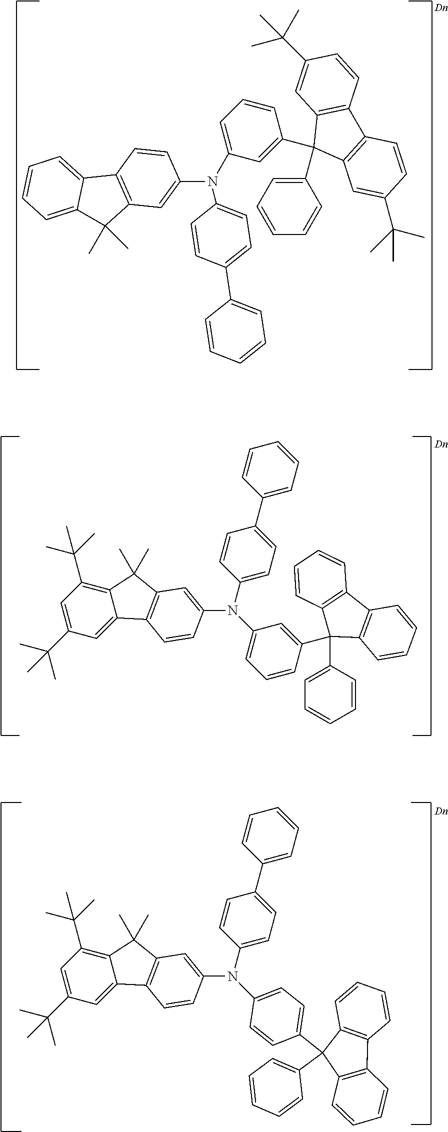

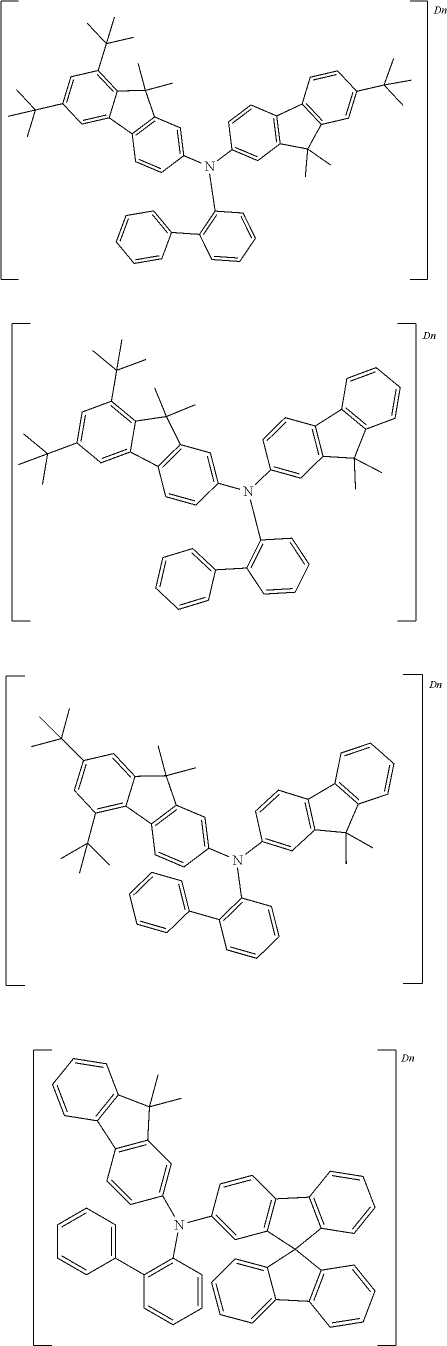

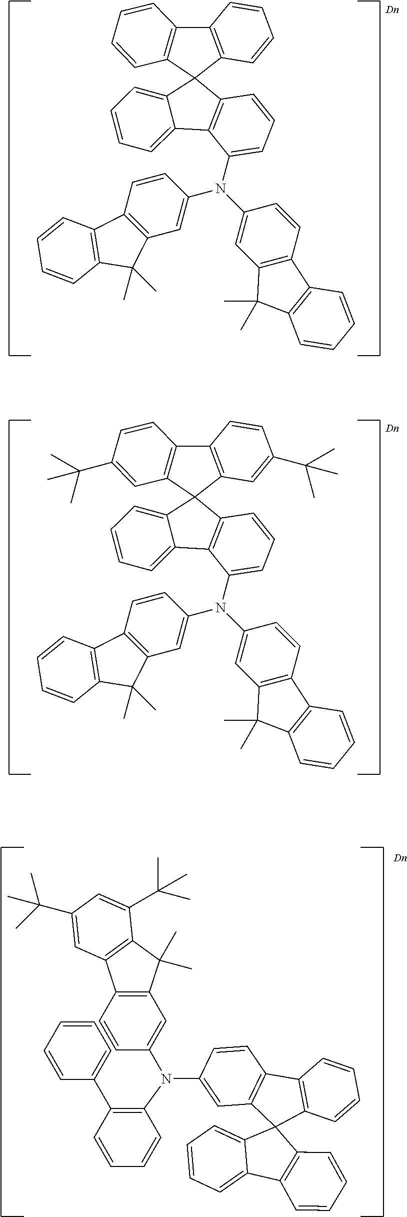

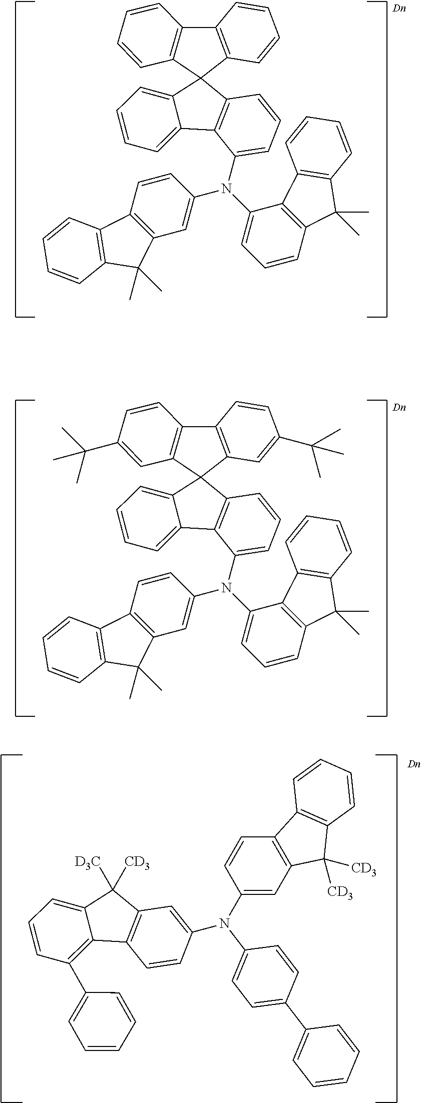

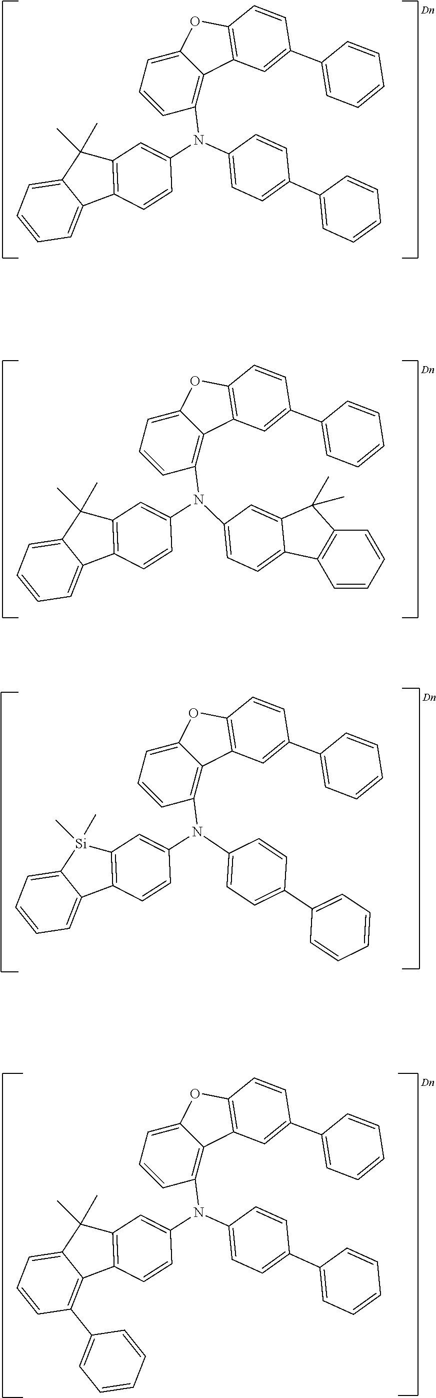

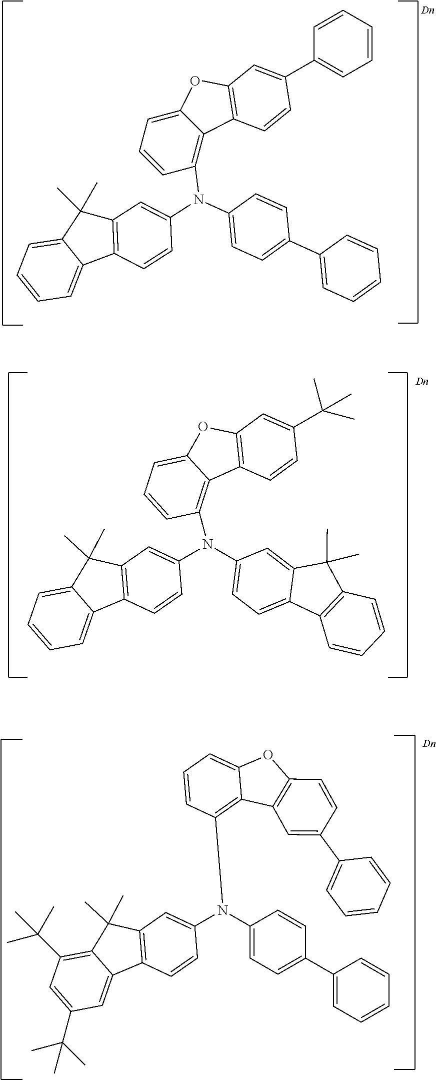

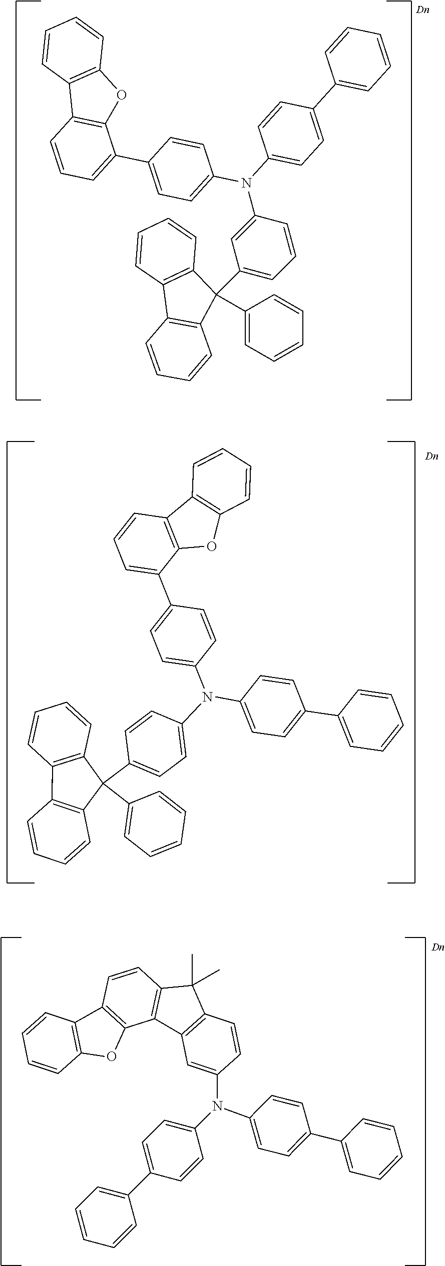

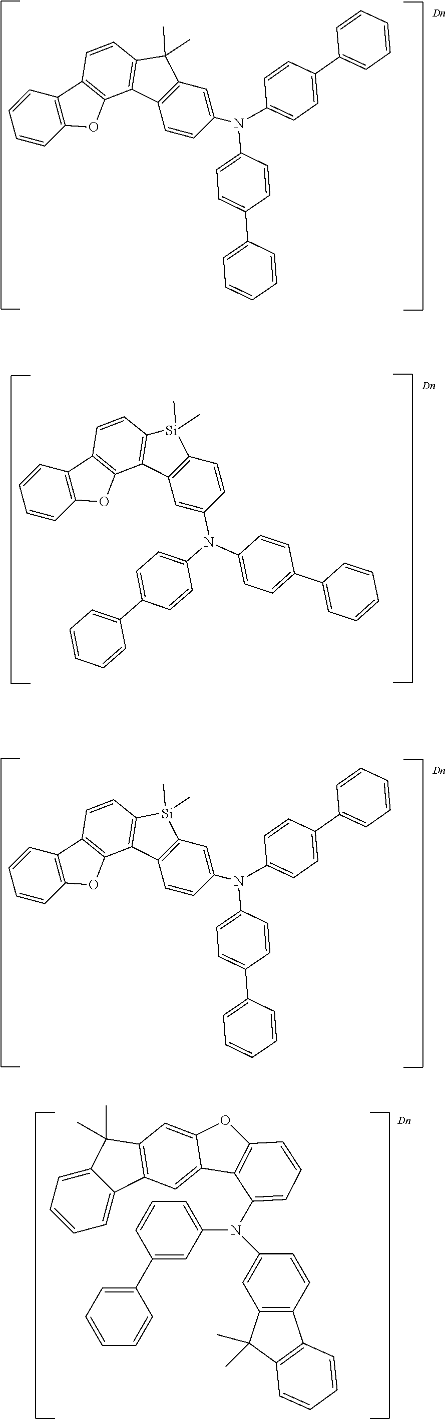

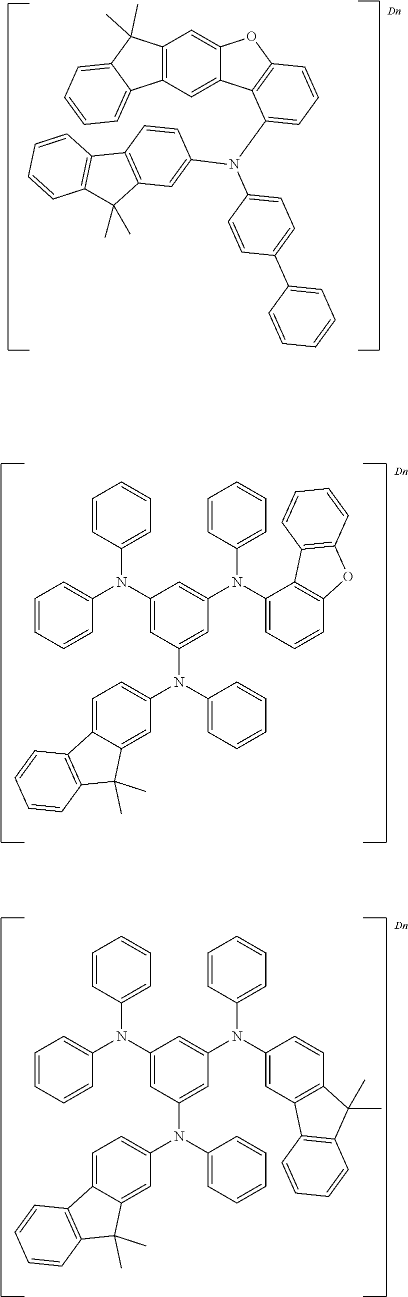

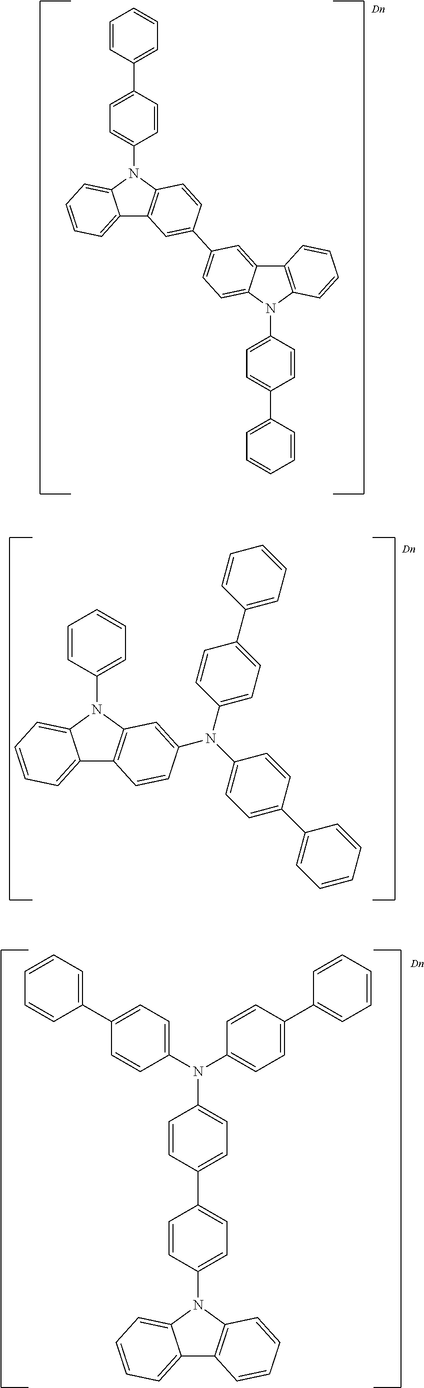

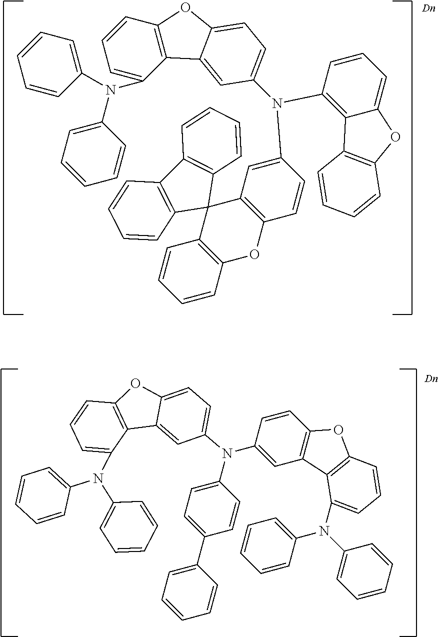

(Dn refers to the number of deuterium atoms substituted, and indicates a structure in which one or more deuterium atoms are substituted)

In the hole transport region 140, in addition to the compounds described above, known compounds disclosed in U.S. Pat. No. 5,061,569A, JP1993-009471A, WO1995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

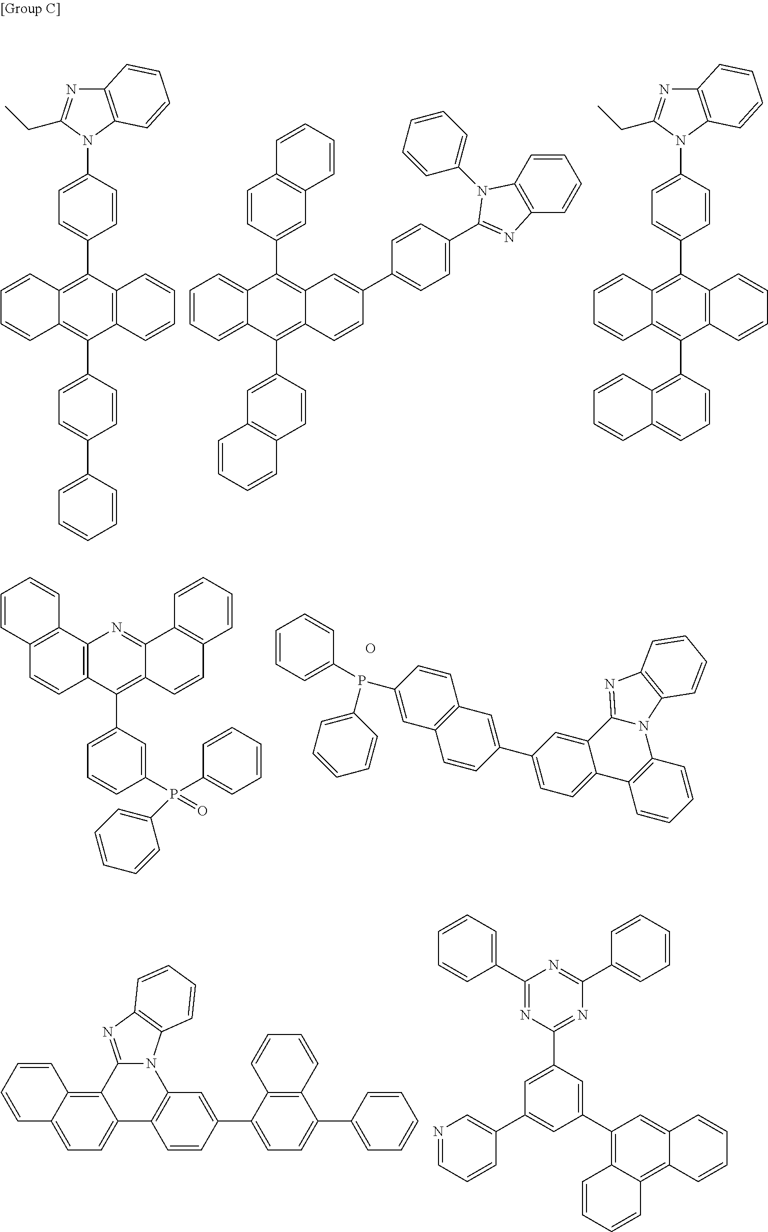

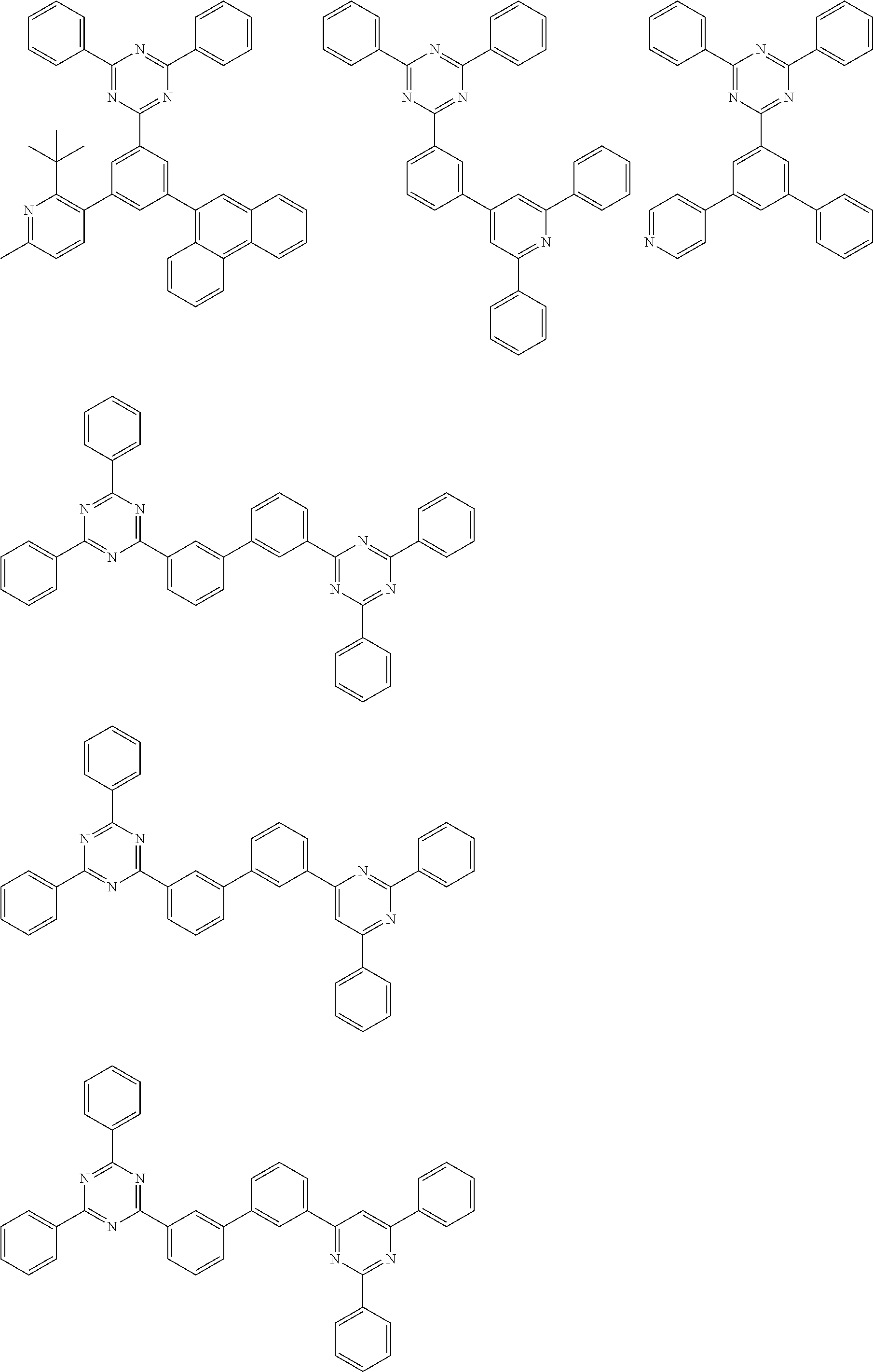

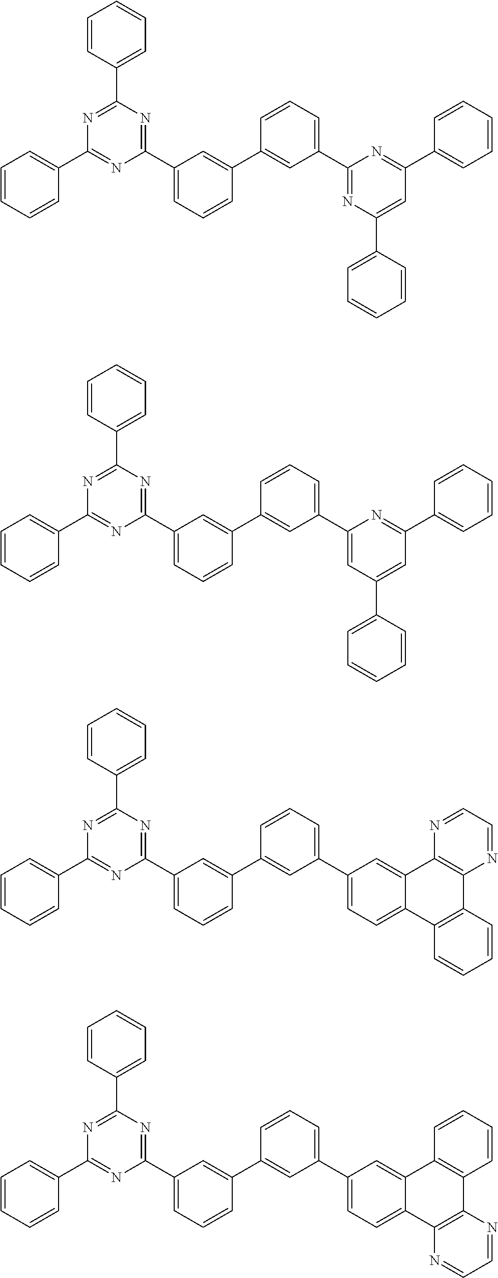 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

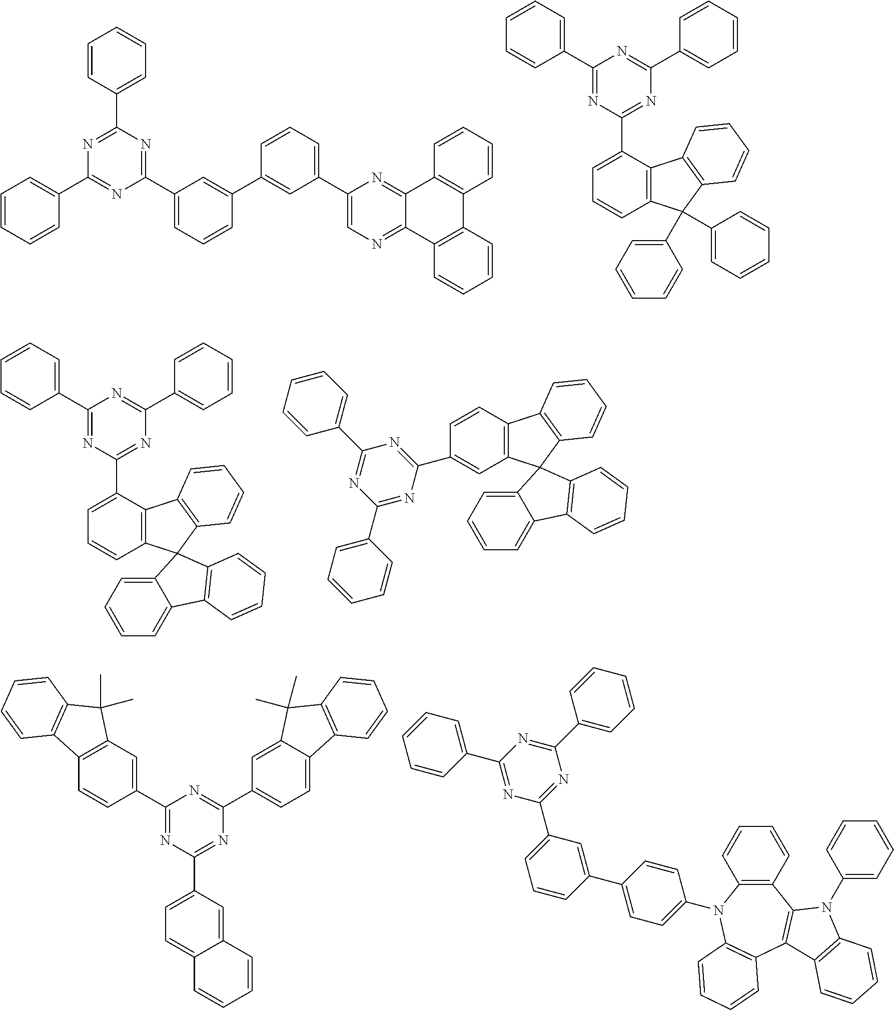

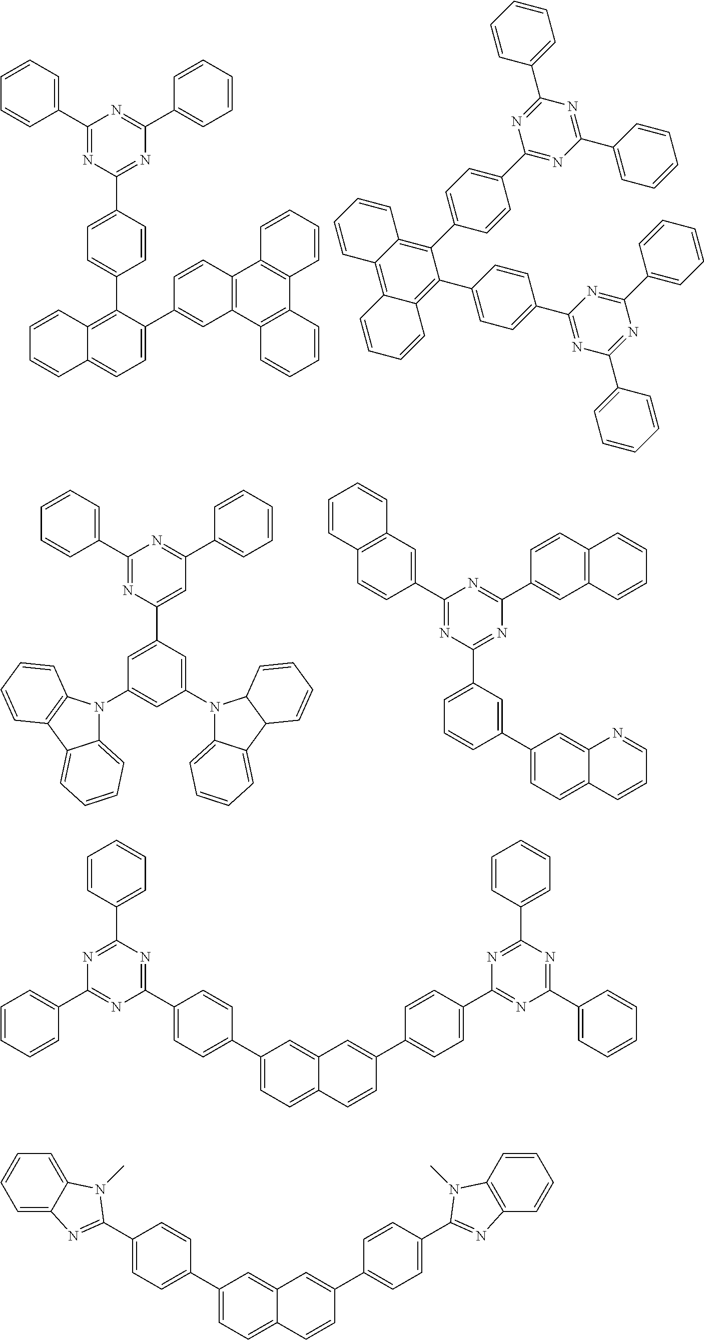

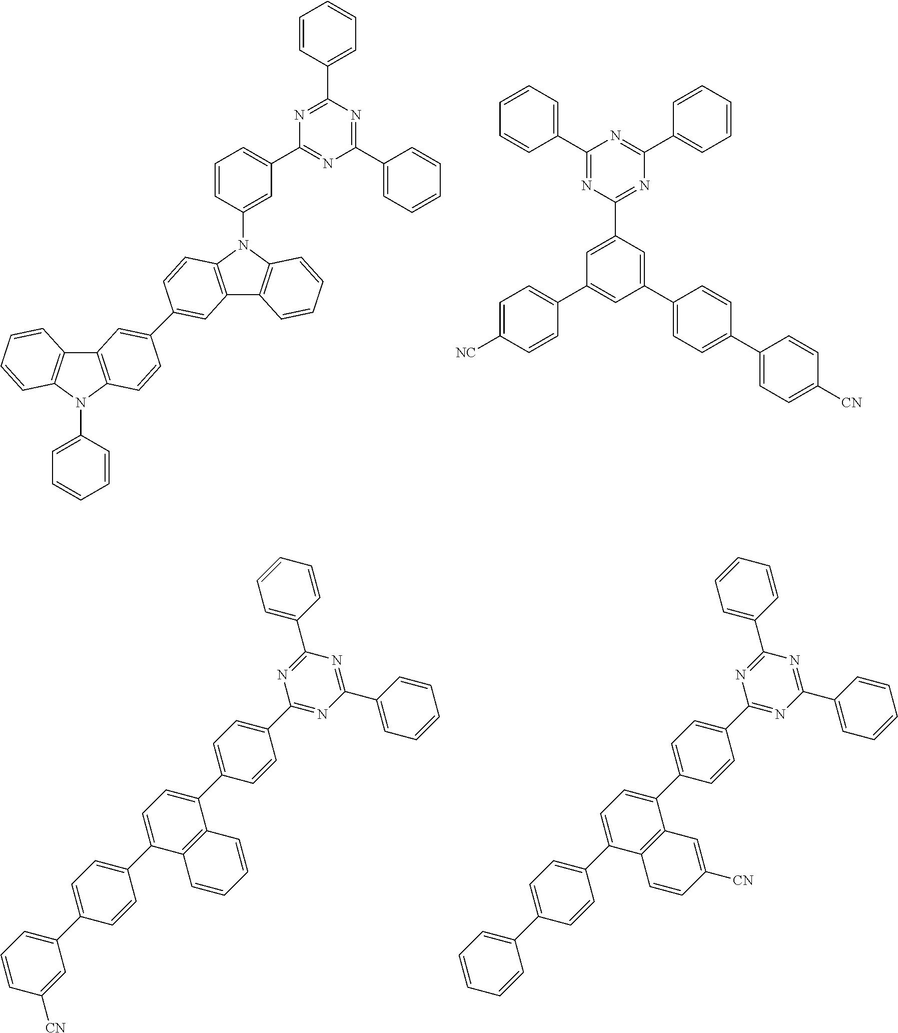 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

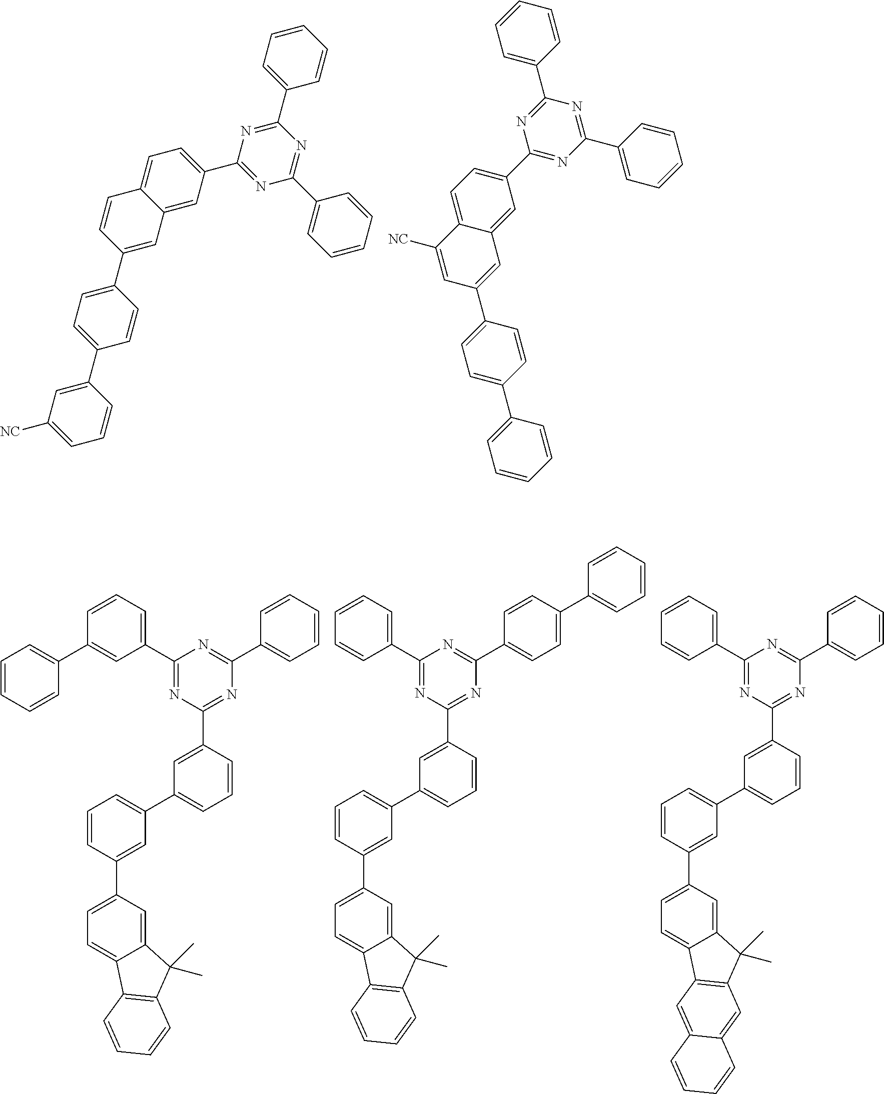

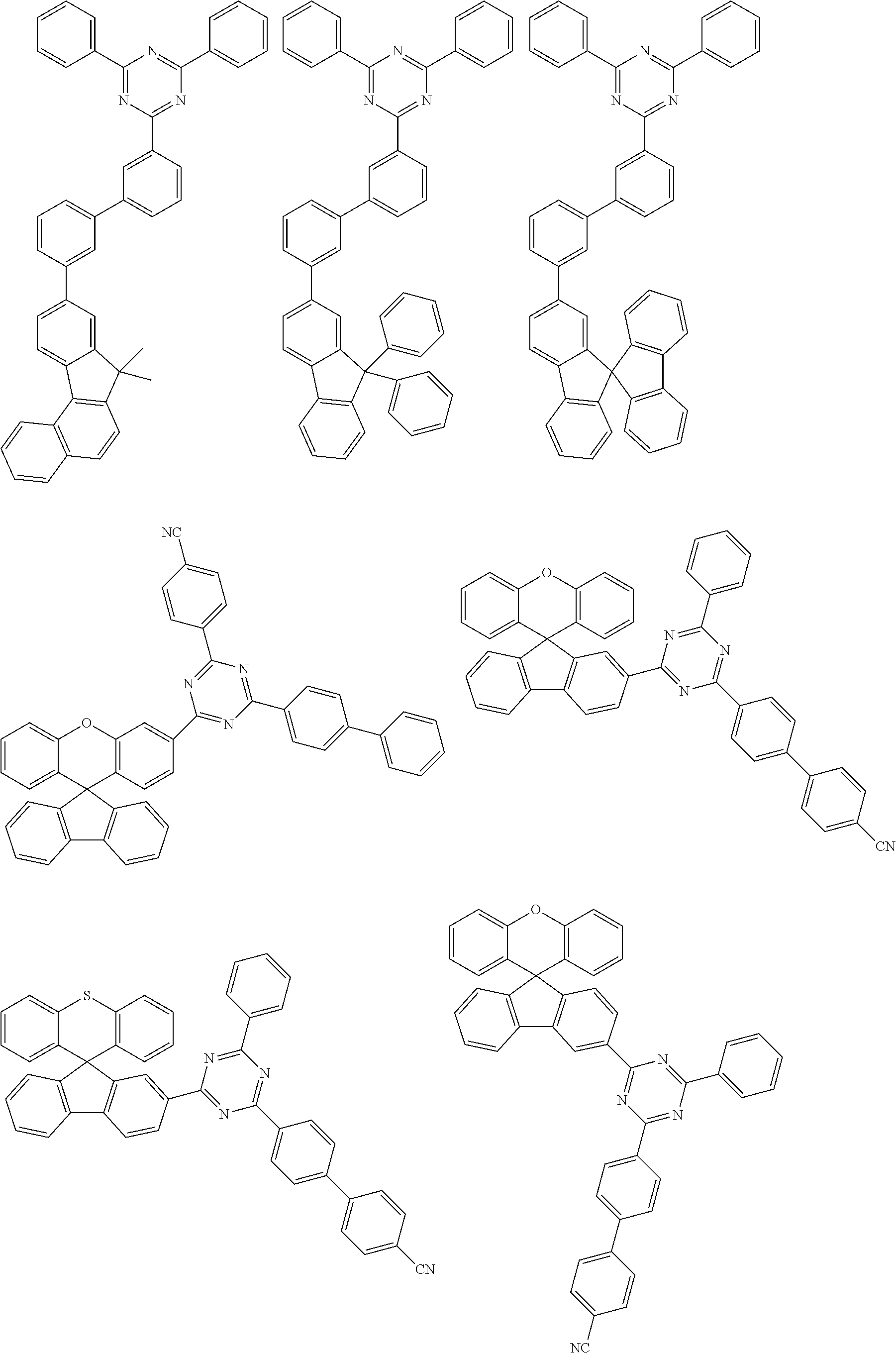

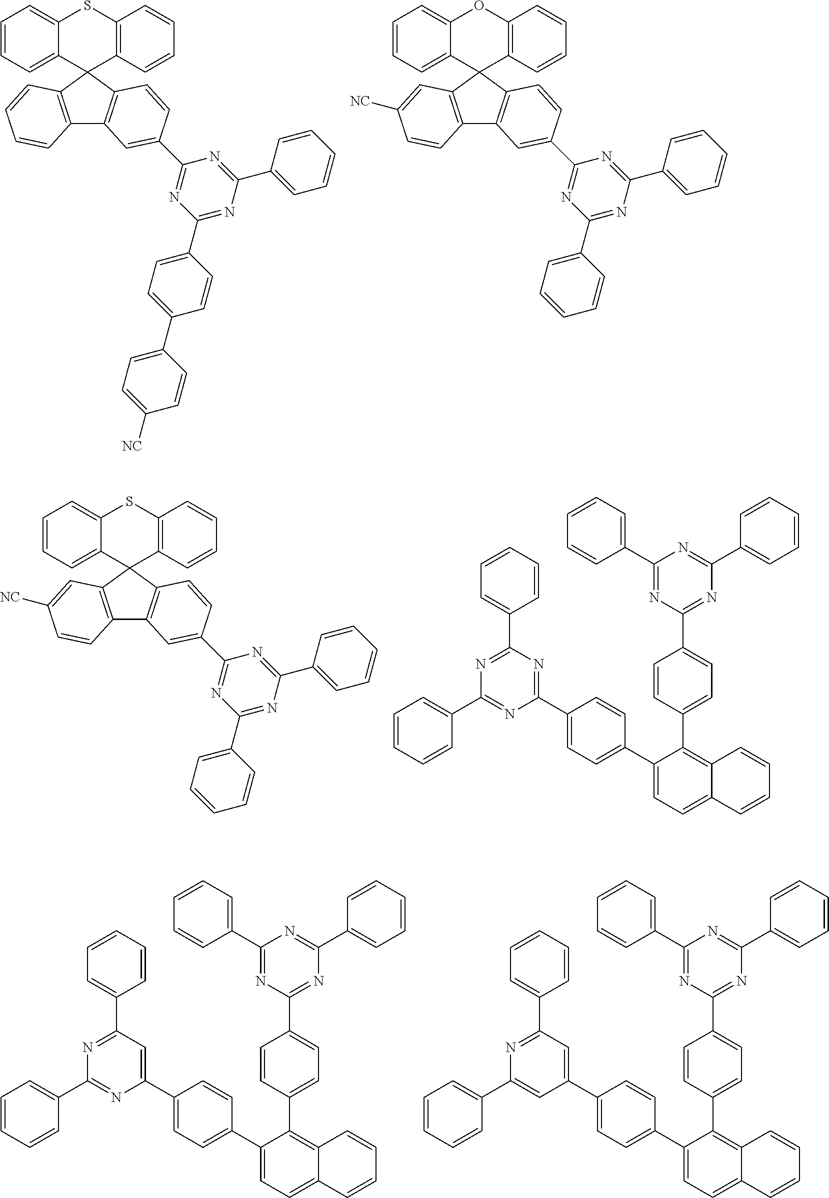

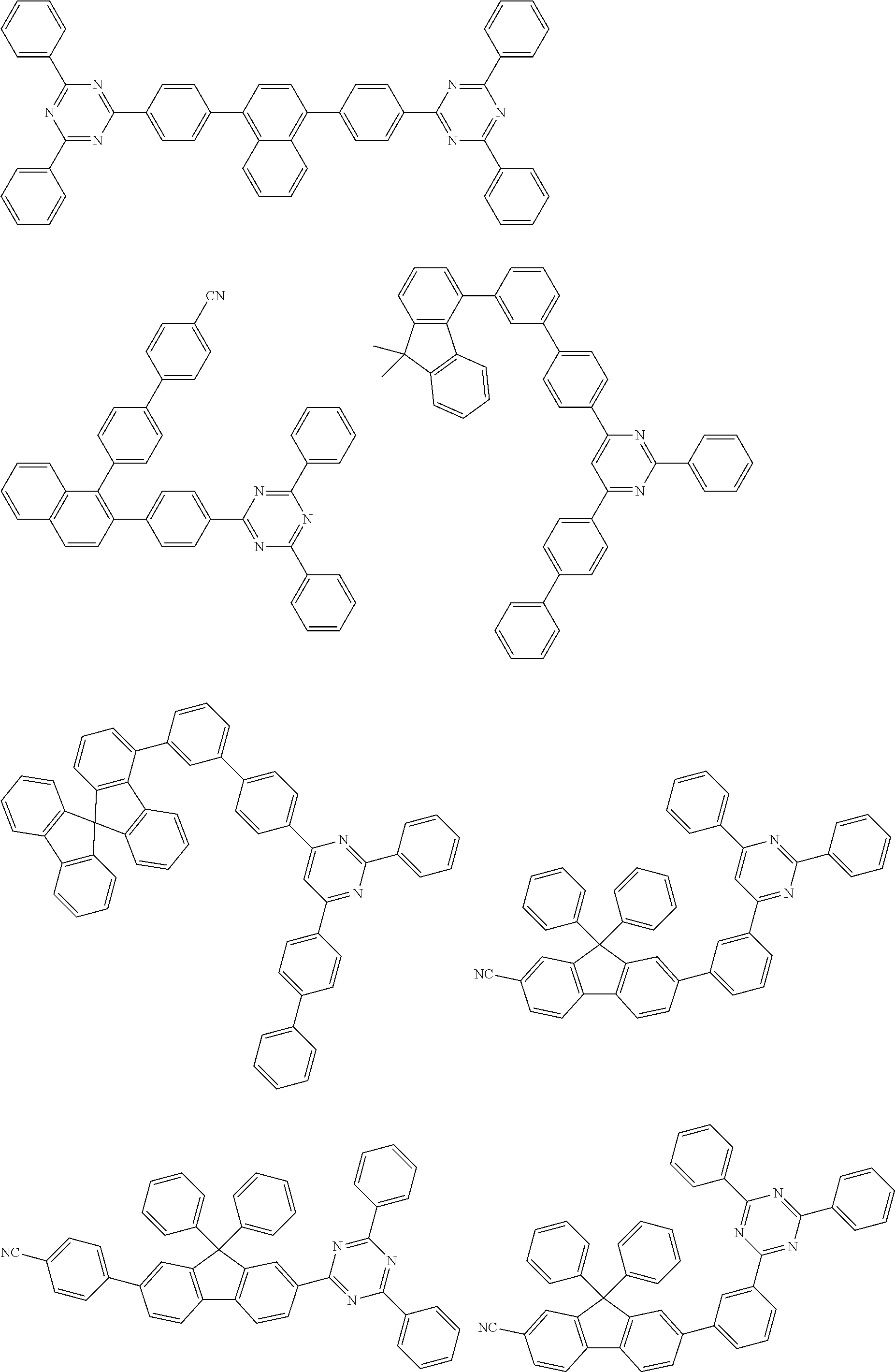

An embodiment may be an organic light emitting diode including the light emitting layer as the organic layer.

Another embodiment may be an organic light emitting diode including a light emitting layer and a hole transport region as the organic layer.

Another embodiment may be an organic light emitting diode including a light emitting layer and an electron transport region as the organic layer.

An organic light emitting diode according to an embodiment includes a hole transport region 140 and an electron transport region 150 in addition to the light emitting layer 130 as the organic layer 105, as shown in FIG. 1.

On the other hand, an organic light emitting diode may further include an electron injection layer (not shown), a hole injection layer (not shown), etc. in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

Hereinafter, the embodiments are illustrated in more detail with reference to examples. However, these examples are exemplary, and the present scope is not limited thereto.

### MODE FOR INVENTION

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by known methods.

### (Preparation of Compound for Organic Optoelectronic Device)

### Synthesis Example 1: Synthesis of Intermediate Int-01

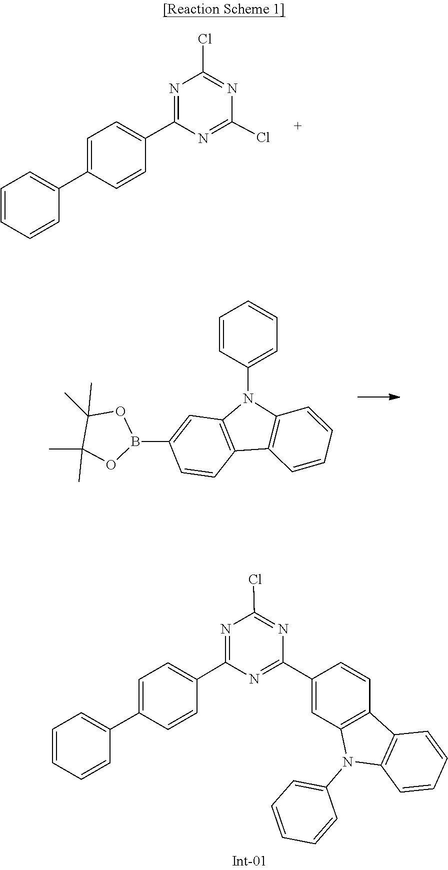 [SMILES: Clc1nc(Cl)nc(-c2ccc(-c3ccccc3)cc2)n1 ; CC1(C)OB(c2ccc3c4ccccc4n(-c4ccccc4)c3c2)OC1(C)C ; Clc1nc(-c2ccc(-c3ccccc3)cc2)nc(-c2ccc3c4ccccc4n(-c4ccccc4)c3c2)n1]

2,4-Dichloro-6-(biphenyl-4-yl)-1,3,5-triazine (61.37 g, 203.11 mmol), 9-phenyl-2-(4,4,5,5-tetramethyl-1,3,2-dioxaborolan-2-yl)-9H-carbazole (50 g, 135.41 mmol), K₂CO₃ (46.79 g, 338.51 mmol), and Pd(dppf)Cl₂ (5.53 g, 6.77 mmol) were added to a round bottom flask and dissolved in toluene (500 ml) and distilled water (200 ml) and then, stirred under reflux at 60° C. for 12 hours. When a reaction was completed, after removing an aqueous layer, a solid obtained therefrom by filtering was dissolved in monochlorobenzene, filtered with silica gel/Celite, and after removing an appropriate amount of the organic solvent, recrystallized with methanol, obtaining 53.07 g (77%) of Intermediate Int-01.

### Synthesis Example 2: Synthesis of Compound A-22-a

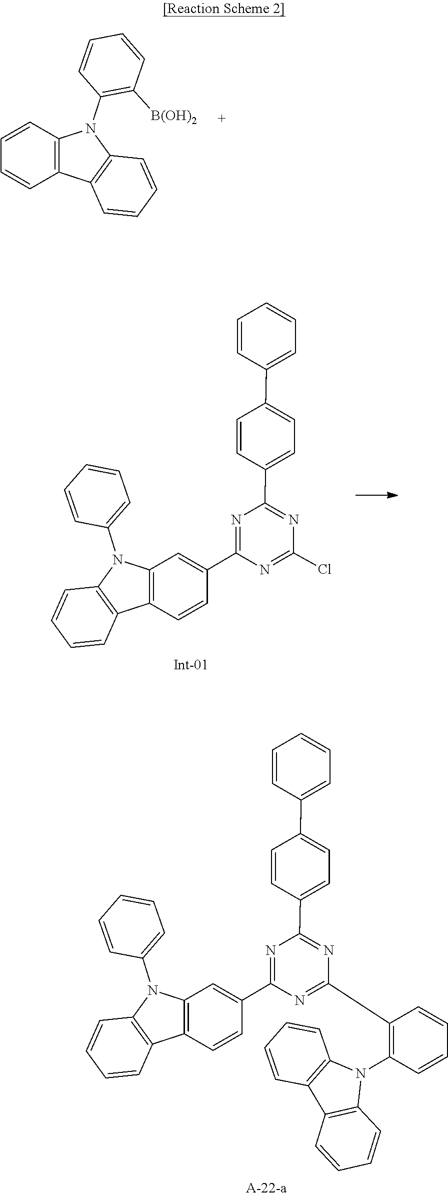 [SMILES: OB(O)c1ccccc1-n1c2ccccc2c2ccccc21 ; Clc1nc(-c2ccc(-c3ccccc3)cc2)nc(-c2ccc3c4ccccc4n(-c4ccccc4)c3c2)n1 ; c1ccc(-c2ccc(-c3nc(-c4ccc5c6ccccc6n(-c6ccccc6)c5c4)nc(-c4ccccc4-n4c5ccccc5c5ccccc54)n3)cc2)cc1]

(2-(9H-carbazol-9-yl)phenyl)boronic acid (8.88 g, 30.94 mmol), Intermediate Int-01 (15 g, 29.47 mmol), K₂CO₃ (10.18 g, 73.67 mmol), and Pd(PPh₃)₄ (1.7 g, 1.47 mmol) were added to a round bottom flask and dissolved in THE (100 ml) and distilled water (40 ml) and then, stirred under reflux at 70° C. for 12 hours. When a reaction was completed, the mixture was added to methanol (500 mL) to crystallize a solid, which was filtered and dissolved in monochlorobenzene and then, filtered with silica gel/Celite and after removing an appropriate amount of the organic solvent, recrystallized with methanol, obtaining 18.4 g (87%) of Compound A-22-a.

### Synthesis Example 3: Synthesis of Intermediate Int-02

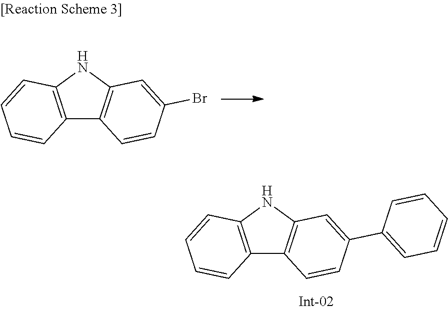 [SMILES: Brc1ccc2c(c1)[nH]c1ccccc12 ; c1ccc(-c2ccc3c(c2)[nH]c2ccccc23)cc1]

2-bromocarbazole (15 g, 60.95 mmol), phenylboronic acid (11.44 g, 73.14 mmol), K₂CO₃ (21.06 g, 152.38 mmol), and Pd(PPh₃)₄ (3.52 g, 3.05 mmol) were added to a round bottom flask and dissolved in THE (200 ml) and distilled water (75 ml) and then, stirred under reflux at 60° C. for 12 hours. When a reaction was completed, after removing an aqueous layer, 12.3 g (83%) of Intermediate Int-02 was obtained therefrom by using column chromatography (hexane:DCM (20%)).

### Synthesis Example 4: Synthesis of Intermediate Int-03

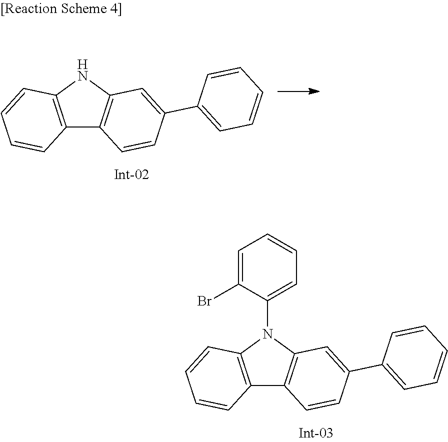 [SMILES: c1ccc(-c2ccc3c(c2)[nH]c2ccccc23)cc1 ; Brc1ccccc1-n1c2ccccc2c2ccc(-c3ccccc3)cc21]

Intermediate Int-02 (15 g, 61.65 mmol), 2-fluorobromobenzene (11.33 g, 64.73 mmol), and K₂PO₄ (28.79 g, 135.64 mmol) were added to a round bottom flask and dissolved in DMF (200 ml) and then, stirred under reflux at 150° C. for 12 hours. When a reaction was completed, water was added thereto and then, stirred for 30 minutes, and after removing an aqueous layer and the organic solvent, 18.2 g (74%) of Intermediate Int-03 was obtained by using column chromatography (hexane:DCM (20%)).

### Synthesis Example 5: Synthesis of Intermediate Int-04

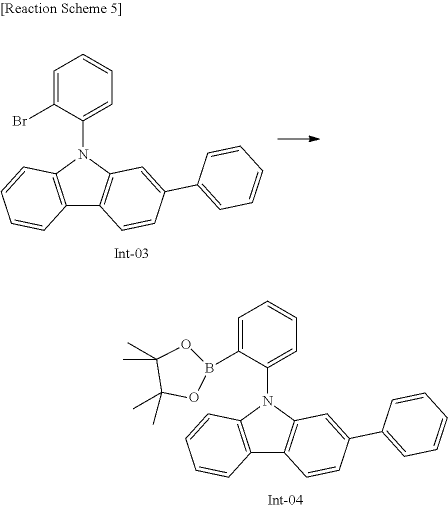 [SMILES: Brc1ccccc1-n1c2ccccc2c2ccc(-c3ccccc3)cc21 ; CC1(C)OB(c2ccccc2-n2c3ccccc3c3ccc(-c4ccccc4)cc32)OC1(C)C]

Intermediate Int-03 (15 g, 37.85 mmol), bis(pinacolato)diboron (12.5 g, 49.21 mmol), Pd(dppf)Cl₂ (1.73 g, 1.9 mmol), and potassium acetate (7.43 g, 75.7 mmol) were added to a round bottom flask and dissolved in toluene (100 ml). The mixture was stirred under reflux at 120° C. for 10 hours. When a reaction was completed, the mixture was poured into an excessive amount of distilled water and then, stirred for 1 hour. A solid was filtered therefrom and dissolved in DCM. After removing moisture with MgSO₄, the organic solvent was filtered with a silica gel pad and then, removed under a reduced pressure. The solid was recrystallized with ethyl acetate and hexane, obtaining 14.6 g (87%) of Intermediate Int-04.

### Synthesis Example 6: Synthesis of Intermediate Int-05

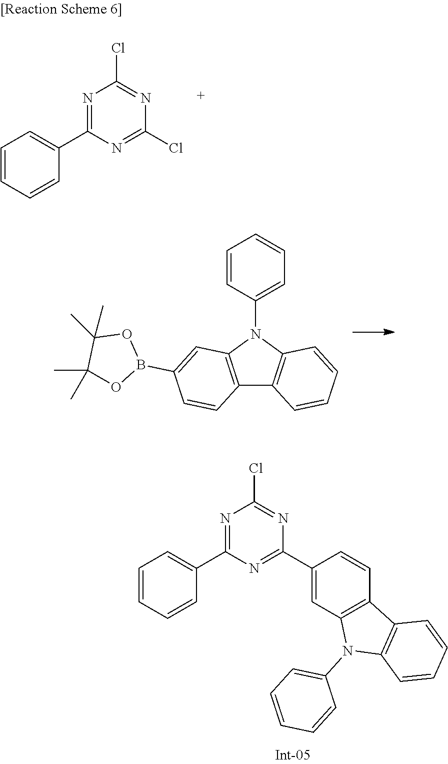 [SMILES: Clc1nc(Cl)nc(-c2ccccc2)n1 ; CC1(C)OB(c2ccc3c4ccccc4n(-c4ccccc4)c3c2)OC1(C)C ; Clc1nc(-c2ccccc2)nc(-c2ccc3c4ccccc4n(-c4ccccc4)c3c2)n1]

15.3 g (76%) of Intermediate Int-05 was obtained in the same manner as in Synthesis Example 1 except that 2,4-dichloro-6-phenyl-1,3,5-triazine was used instead of the 2,4-dichloro-6-(biphenyl-4-yl)-1,3,5-triazine.

### Synthesis Example 7: Synthesis of Compound A-32-a

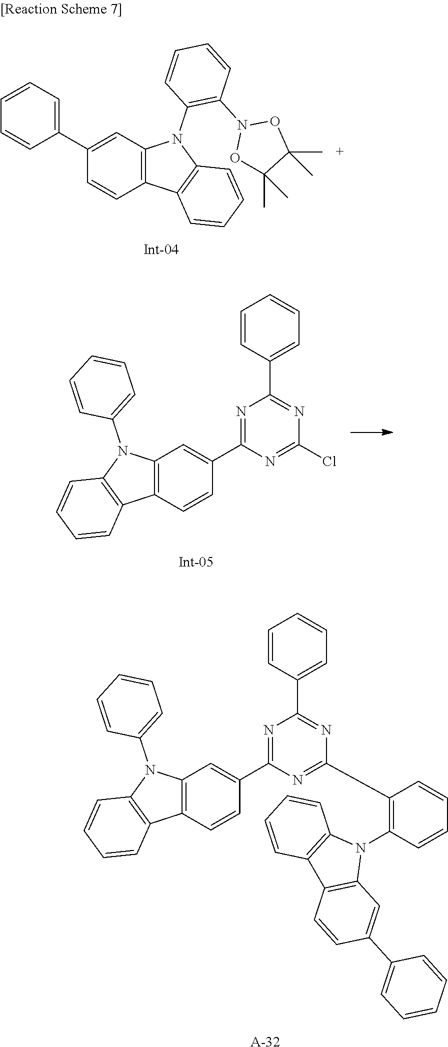 [SMILES: CC1(C)ON(c2ccccc2-n2c3ccccc3c3ccc(-c4ccccc4)cc32)OC1(C)C ; Clc1nc(-c2ccccc2)nc(-c2ccc3c4ccccc4n(-c4ccccc4)c3c2)n1 ; c1ccc(-c2ccc3c4ccccc4n(-c4ccccc4-c4nc(-c5ccccc5)nc(-c5ccc6c7ccccc7n(-c7ccccc7)c6c5)n4)c3c2)cc1 ; "A-32"]

17.1 g (78%) of Compound A-32-a was obtained in the same manner as in Synthesis Example 2 except that Intermediate Int-04 and Intermediate Int-05 were used instead of the (2-(9H-carbazol-9-yl)phenyl)boronic acid and Intermediate Int-01.

### Synthesis Example 8: Synthesis of Intermediate Int-06

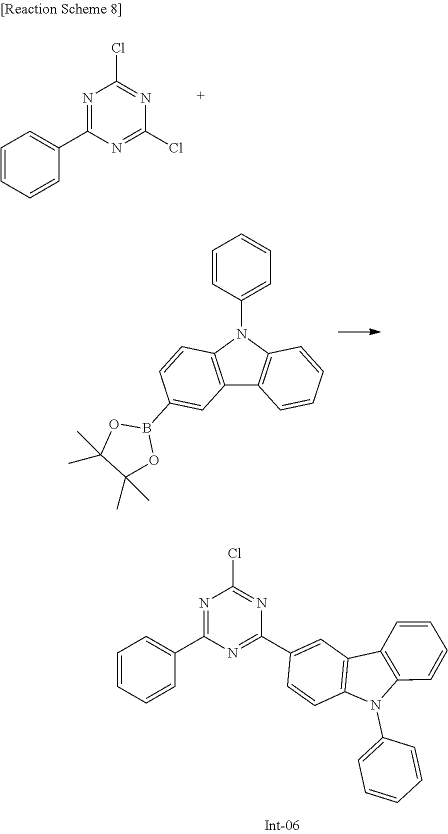 [SMILES: Clc1nc(Cl)nc(-c2ccccc2)n1 ; CC1(C)OB(c2ccc3c(c2)c2ccccc2n3-c2ccccc2)OC1(C)C ; Clc1nc(-c2ccccc2)nc(-c2ccc3c(c2)c2ccccc2n3-c2ccccc2)n1]

11.8 g (71%) of Intermediate Int-06 was obtained in the same manner as in Synthesis Example 1 except that 2,4-dichloro-6-phenyl-1,3,5-triazine and 9-phenyl-3-(4,4,5,5-tetramethyl-1,3,2-dioxaborolan-2-yl)-9H-carbazole were used instead of the 2,4-dichloro-6-(biphenyl-4-yl)-1,3,5-triazine and the 9-phenyl-2-(4,4,5,5-tetramethyl-1,3,2-dioxaborolan-2-yl)-9H-carbazole.

### Synthesis Example 9: Synthesis of Compound A-57-a

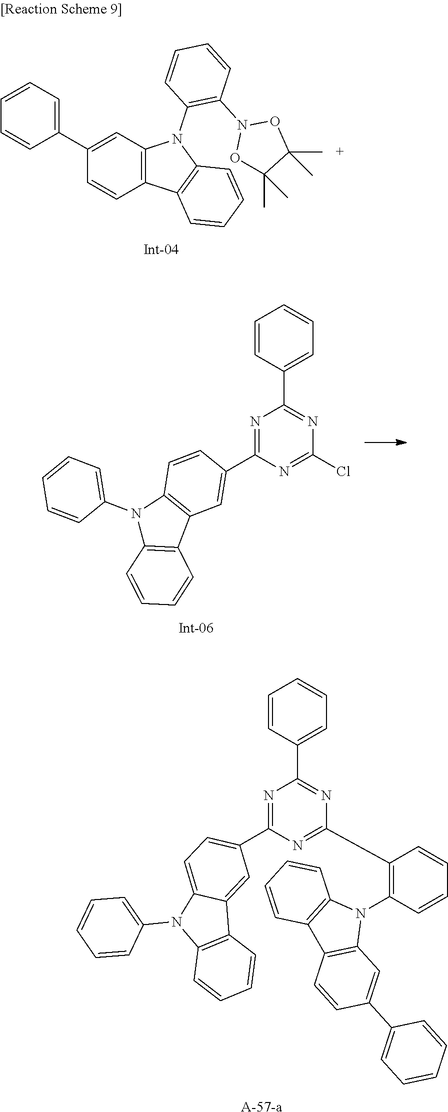 [SMILES: CC1(C)ON(c2ccccc2-n2c3ccccc3c3ccc(-c4ccccc4)cc32)OC1(C)C ; Clc1nc(-c2ccccc2)nc(-c2ccc3c(c2)c2ccccc2n3-c2ccccc2)n1 ; c1ccc(-c2ccc3c4ccccc4n(-c4ccccc4-c4nc(-c5ccccc5)nc(-c5ccc6c(c5)c5ccccc5n6-c5ccccc5)n4)c3c2)cc1 ; "A-57-a"]

8.7 g (79%) of Compound A-57-a was obtained in the same manner as in Synthesis Example 2 except that Intermediate Int-04 and Intermediate Int-06 were used instead of the (2-(9H-carbazol-9-yl)phenyl)boronic acid and Intermediate Int-01.

### Synthesis Example 10: Synthesis of Intermediate Int-07

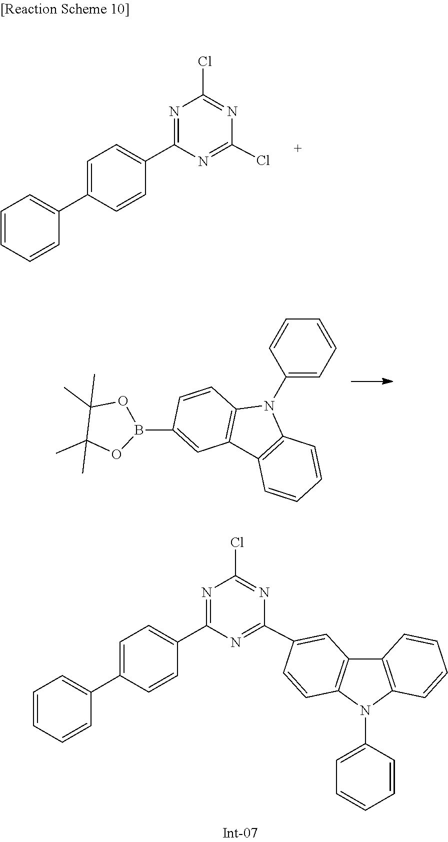 [SMILES: Clc1nc(Cl)nc(-c2ccc(-c3ccccc3)cc2)n1 ; CC1(C)OB(c2ccc3c(c2)c2ccccc2n3-c2ccccc2)OC1(C)C ; Clc1nc(-c2ccc(-c3ccccc3)cc2)nc(-c2ccc3c(c2)c2ccccc2n3-c2ccccc2)n1]

14.3 g (73%) of Intermediate Int-07 was obtained in the same manner as in Synthesis Example 1 except that 9-phenyl-3-(4,4,5,5-tetramethyl-1,3,2-dioxaborolan-2-yl)-9H-carbazole was used instead of the 9-phenyl-2-(4,4,5,5-tetramethyl-1,3,2-dioxaborolan-2-yl)-9H-carbazole.

### Synthesis Example 11: Synthesis of Compound A-47-a

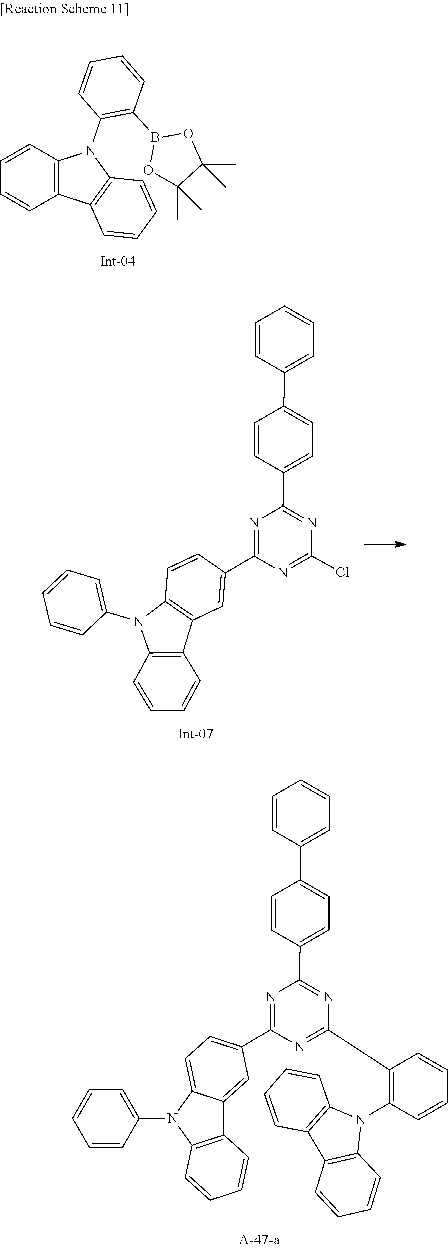 [SMILES: CC1(C)OB(c2ccccc2-n2c3ccccc3c3ccccc32)OC1(C)C ; Clc1nc(-c2ccc(-c3ccccc3)cc2)nc(-c2ccc3c(c2)c2ccccc2n3-c2ccccc2)n1 ; c1ccc(-c2ccc(-c3nc(-c4ccc5c(c4)c4ccccc4n5-c4ccccc4)nc(-c4ccccc4-n4c5ccccc5c5ccccc54)n3)cc2)cc1]

7.4 g (81%) of Compound A-47-a was obtained in the same manner as in Synthesis Example 2 except that Intermediate Int-04 and Intermediate Int-07 were used instead of the (2-(9H-carbazol-9-yl)phenyl)boronic acid and Intermediate Int-01.

### Synthesis Example 12: Synthesis of Compound A-22

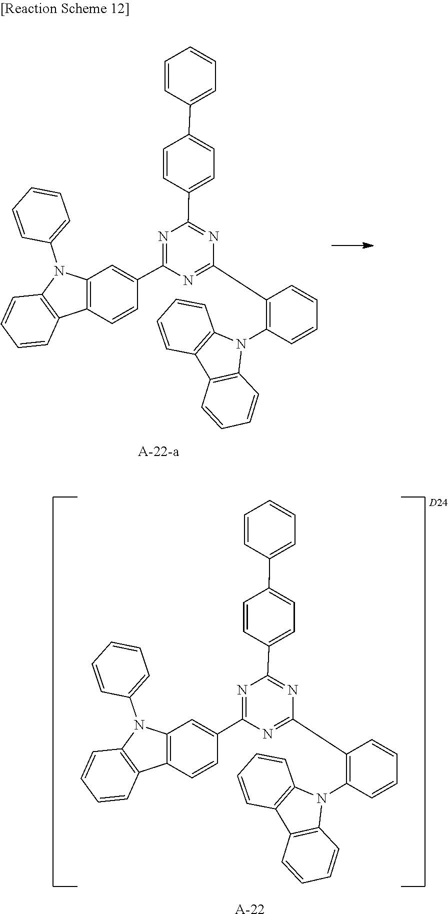

20 g of Compound A-22-a, 15 ml of trifluoromethanesulfonic acid, and 350 ml of D₆-benzene were added to a flask and stirred at 70° C. for 24 hours. After adding purified water thereto and neutralizing the mixture with a saturation K₃PO₄ solution, an organic layer therefrom was concentrated and column-purified, obtaining Compound A-22 (yield: 14 g (70%), the number of substitutions: D24).

### Synthesis Example 13: Synthesis of Compound A-32

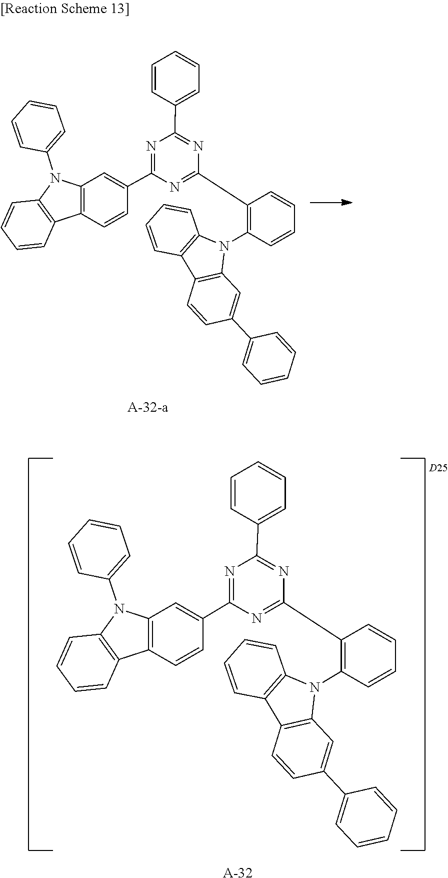

Compound A-32 (yield: 13.2 g (68%), the number of substitutions: D25) was obtained in the same manner as in Synthesis Example 12 except that Compound A-32-a was used instead of Compound A-22-a.

### Synthesis Example 14: Synthesis of Compound A-57

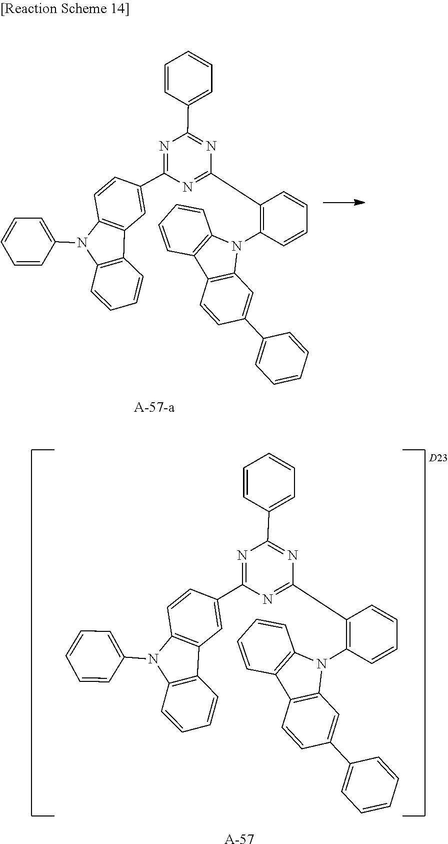

Compound A-57 (yield: 17.2 g (69%), the number of substitutions: D23) was obtained in the same manner as in Synthesis Example 12 except that Compound A-57-a was used instead of Compound A-22-a.

### Synthesis Example 15: Synthesis of Compound A-47

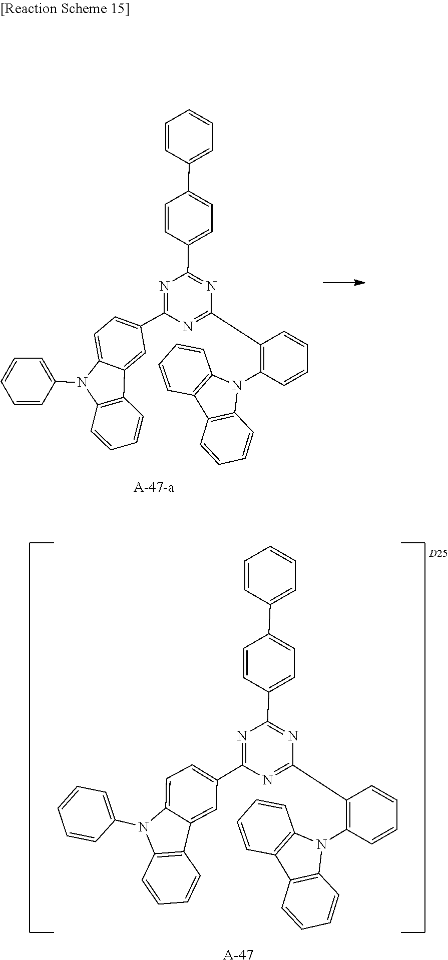

Compound A-47 (yield: 19.2 g (83%), the number of substitutions: D25) was obtained in the same manner as in Synthesis Example 12 except that Compound A-47-a was used instead of Compound A-22-a.

### Synthesis Example 16: Synthesis of Compound B-1

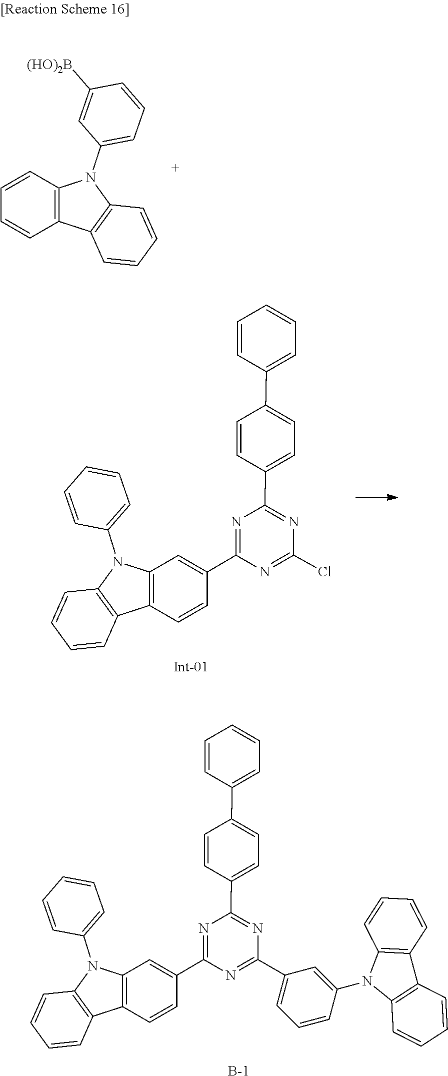

Compound B-1 (yield: 5 g (67%), the number of substitutions: D25) was obtained in the same manner as in Synthesis Example 2 except that (3-(9H-carbazol-9-yl)phenyl)boronic acid was used instead of the (2-(9H-carbazol-9-yl)phenyl)boronic acid.

### Synthesis Example 17: Synthesis of Compound B-2

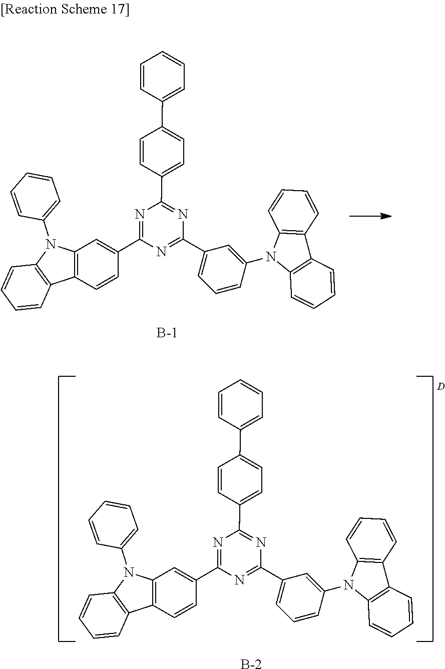

Compound B-2 (yield: 15.3 g (78.1%), the number of substitutions: D23) was obtained in the same manner as in Synthesis Example 12 except that Compound B-1 was used instead of Compound A-22-a.

### Example 1

A glass substrate coated with ITO (Indium tin oxide) was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited to on the hole injection layer to form a 1350 Å-thick hole transport layer. Compound B was deposited on the hole transport layer to a thickness of 350 Å to form a hole transport auxiliary layer. Compound A-22 obtained in Synthesis Example 12 was used as a host on the hole transport auxiliary layer, and 7 wt % of PhGD was doped as a dopant to form a 400 Å-thick light emitting layer by vacuum deposition. Next, Compound C was deposited on the light emitting layer to a thickness of 50 Å to form an electron transport auxiliary layer, and Compound D and LiQ were simultaneously vacuum deposited at a weight ratio of 1:1 to form a 300 Å-thick electron transport layer. An organic light emitting diode was manufactured by sequentially vacuum-depositing 15 Å of LiQ and 1200 Å of Al on the electron transport layer to form a cathode.

The organic light emitting diode has a structure of ITO/Compound A (3% NDP-9 doping, 100 Å)/Compound A (1350 Å)/Compound B (350 Å)/EML [93 wt % of host (Compound A-22): 7 wt % of PhGD](400 Å) Compound C (50 Å)/Compound D:LiQ (300 Å)/LiQ (15 Å)/Al (1200 Å).

Compound A: N-(biphenyl-4-yl)-9,9-dimethyl-N-(4-(9-phenyl-9H-carbazol-3-yl)phenyl)-9H-fluoren-2-amine

Compound B: N-[4-(4-dibenzofuranyl)phenyl]-N-[4-(9-phenyl-9H-fluoren-9-yl)phenyl][1,1′-biphenyl]-4-amine

Compound C: 2,4-diphenyl-6-(4′,5′,6′-triphenyl[1,1′:2′,1″:3″,1′″:3′″,1″″-quinquephenyl]-3″″-yl)-1,3,5-triazine

Compound D: 2-(1,1′-biphenyl-4-yl)-4-(9,9-diphenylfluoren-4-yl)-6-phenyl-1,3,5-triazine

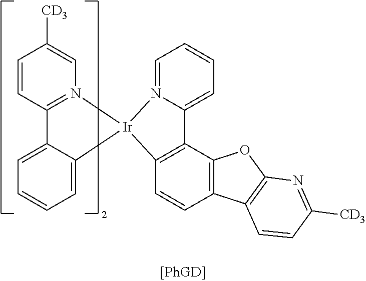

### Examples 2 to 4 and Comparative Examples 1 to 2

Each organic light emitting diode was manufactured in the same manner as in Example 1, except that the compositions were changed to that described in Tables 1 and 2.

### Evaluation

The luminous efficiency and life-span characteristics of the organic light emitting diodes according to Examples 1 to 4 and Comparative Examples 1 to 2 were evaluated.

The specific measurement method is as follows, and the results are as shown in Tables 1 and 2.

### (1) Measurement of Current Density Change Depending on Voltage Change

The obtained organic light emitting diodes were measured regarding a current value flowing in the unit diode, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Luminance was measured by using a luminance meter (Minolta Cs-1000A), while the voltage of the organic light emitting diodes was increased from 0 V to 10 V.

The luminous efficiency values of Examples 1 to 4 and Comparative Example 2 were calculated as relative values based on Example 1 and are listed in Table 2.

### (4) Measurement of Life-Span

Time when each current efficiency (cd/A) was reduced to 97%, while maintaining luminance (cd/m²) at 24000 cd/m², was measured as a life-span.

The life-span measurement values of Example 1 and Comparative Example 1 were calculated as relative values based on Example 1 and are listed in Table 1, and
the life-span measurement values of Examples 1 to 4 and Comparative Example 2 were calculated as relative values based on Example 1 and are listed in Table 2.

TABLE 1

| No. | Host | Life-span (%) |
|---|---|---|
| Example 1 | A-22 | 100 |
| Comparative Example 1 | A-22-a | 82 |

TABLE 2

| No. | Host | Efficiency (%) | Life-span (%) |
|---|---|---|---|
| Example 1 | A-22 | 100 | 100 |
| Example 2 | A-32 | 98 | 110 |
| Example 3 | A-47 | 98 | 90 |
| Example 4 | A-57 | 97 | 95 |
| Comparative Example 2 | B-2 | 93 | 65 |

Referring to Table 1, Example 1 is a D-substituted structure of the structure of Comparative Example 1, and as can be seen from the diode data, the life-span characteristics are increased by approximately 18%. From this, it can be confirmed that the stability of the compound greatly increases when D is substituted, and thus the life-span is significantly increased.

In addition, referring to Table 2, the structure of Comparative Example 2 is a structure including a meta linker instead of an ortho linker, and the structure including the ortho linker in the D-substituted structure, as in Examples 1 to 4, further increases the efficiency and life-span characteristics. From this, structural steric hindrance is created through the ortho linker, allowing holes and electrons to flow better during deposition, thereby increasing efficiency and life-span characteristics.

1. A compound for an organic optoelectronic device represented by Chemical Formula 1: 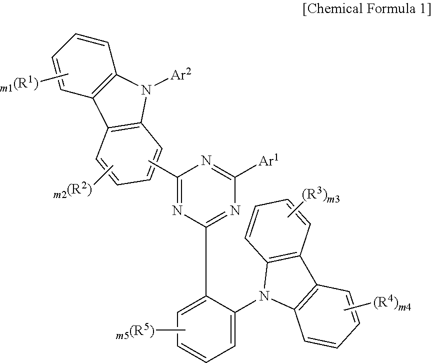
wherein, in Chemical Formula 1,
Ar¹ and Ar² are each independently a substituted or unsubstituted C6 to C20 aryl group or a substituted or unsubstituted C2 to C20 heterocyclic group,
R¹ to R⁵ are each independently hydrogen, deuterium, a cyano group, a substituted or unsubstituted C1 to C10 alkyl group, or a substituted or unsubstituted C6 to C12 aryl group,
m1, and m3 to m5 are each independently one of integers of 1 to 4,
m2 is one of integers of 1 to 3, and
Chemical Formula 1 satisfies at least one of the following (i) to (iii):
(i) at least one of R¹ to R⁵ is deuterium, a C1 to C10 alkyl group substituted with one or more deuterium atoms, or a C6 to C12 aryl group substituted with one or more deuterium atoms;
(ii) Ar¹ is a C6 to C20 aryl group substituted with one or more deuterium atoms or a C2 to C30 heterocyclic group substituted with one or more deuterium atoms; and
(iii) Ar² is a C6 to C20 aryl group substituted with one or more deuterium atoms or a C2 to C30 heterocyclic group substituted with one or more deuterium atoms.

2. The compound for the organic optoelectronic device as claimed in claim 1, wherein:
at least one of R¹ to R⁵ is deuterium, a C1 to C10 alkyl group substituted with one or more deuterium atoms, or a C6 to C12 aryl group substituted with one or more deuterium atoms, and
Ar¹ is a C6 to C20 aryl group substituted with one or more deuterium atoms or a C2 to C30 heterocyclic group substituted with one or more deuterium atoms.

3. The compound for the organic optoelectronic device as claimed in claim 1, wherein:
at least one of R¹ to R⁵ is deuterium, a C1 to C10 alkyl group substituted with one or more deuterium atoms, or a C6 to C12 aryl group substituted with one or more deuterium atoms, and
Ar² is a C6 to C20 aryl group substituted with one or more deuterium atoms or a C2 to C30 heterocyclic group substituted with one or more deuterium atoms.

4. The compound for the organic optoelectronic device as claimed in claim 1, wherein:
at least one of R¹ to R⁵ is deuterium, a C1 to C10 alkyl group substituted with one or more deuterium atoms, or a C6 to C12 aryl group substituted with one or more deuterium atoms,
Ar¹ is a C6 to C20 aryl group substituted with one or more deuterium atoms or a C2 to C30 heterocyclic group substituted with one or more deuterium atoms, and
Ar² is a C6 to C20 aryl group substituted with one or more deuterium atoms or a C2 to C30 heterocyclic group substituted with one or more deuterium atoms.

5. The compound for the organic optoelectronic device as claimed in claim 1, wherein a deuterium substitution ratio of Chemical Formula 1 is 10% to 100%.

6. The compound for the organic optoelectronic device as claimed in claim 1, wherein:
Chemical Formula 1 is represented by any one of Chemical Formula 1A to Chemical Formula 1D: 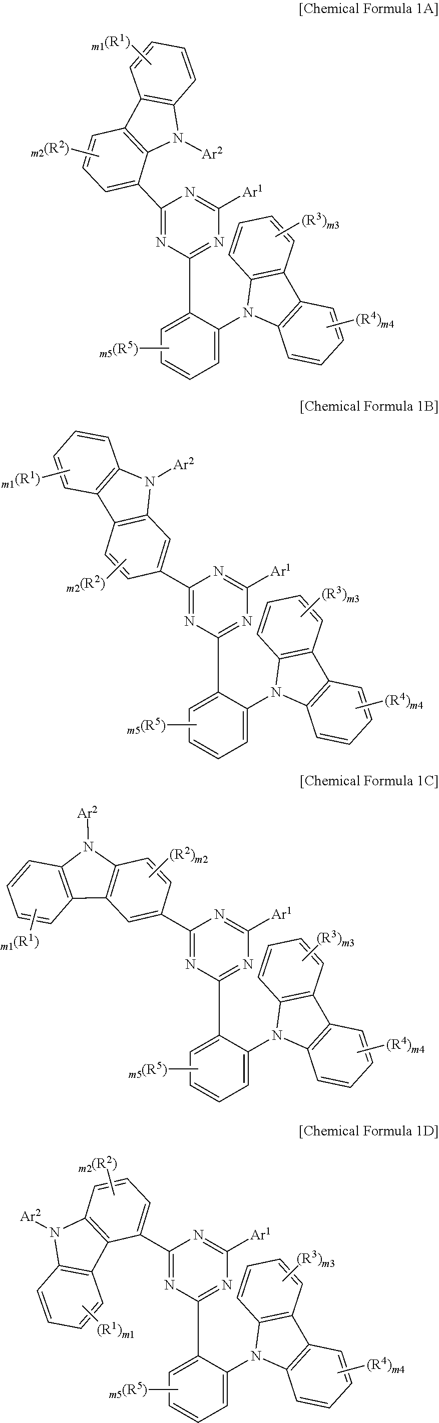
wherein, in Chemical Formula 1A to Chemical Formula 1D, R¹ to R⁵, Ar¹, Ar², and m1 to m5 are defined the same as those of Chemical Formula 1.

7. The compound for the organic optoelectronic device as claimed in claim 1, wherein in Chemical Formula 1, Ar¹ is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted fluorenyl group, a substituted or unsubstituted dibenzofuranyl group, a substituted or unsubstituted dibenzothiophenyl group, or a substituted or unsubstituted dibenzosilolyl group.

8. The compound for the organic optoelectronic device as claimed in claim 1, wherein
the first compound is selected from the compounds listed in Group 1: 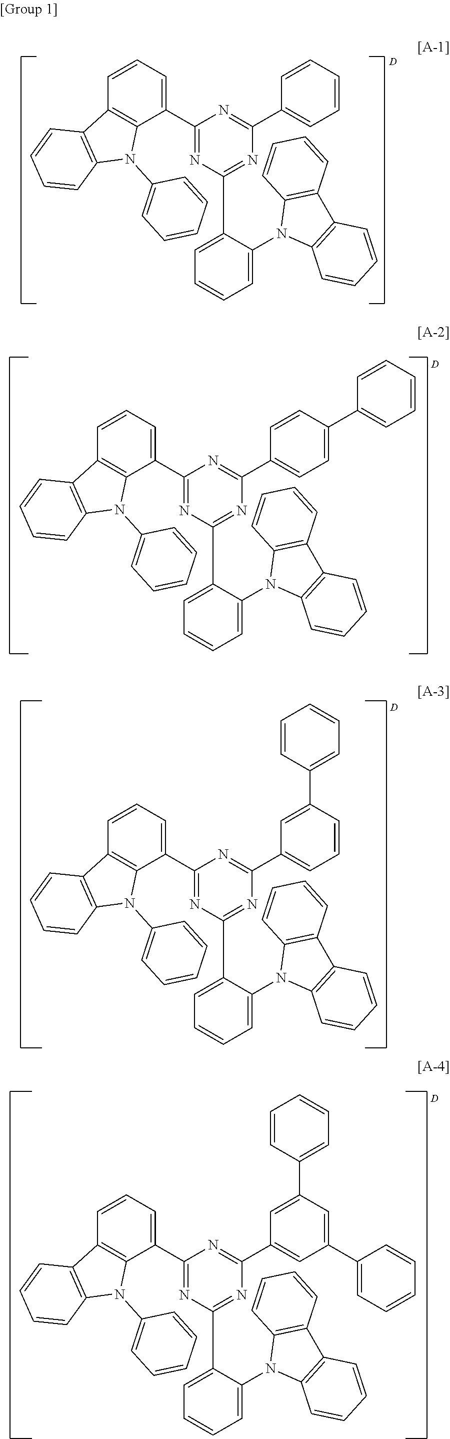 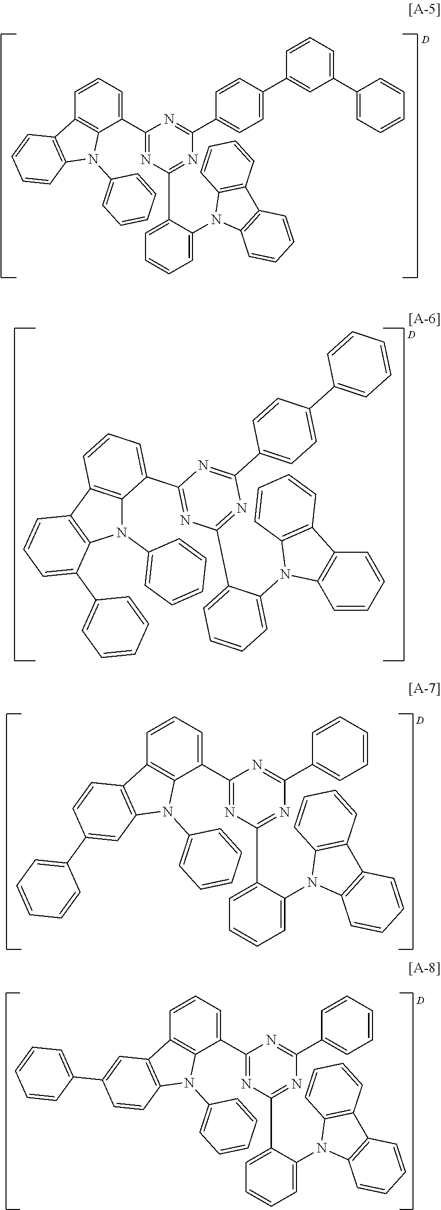 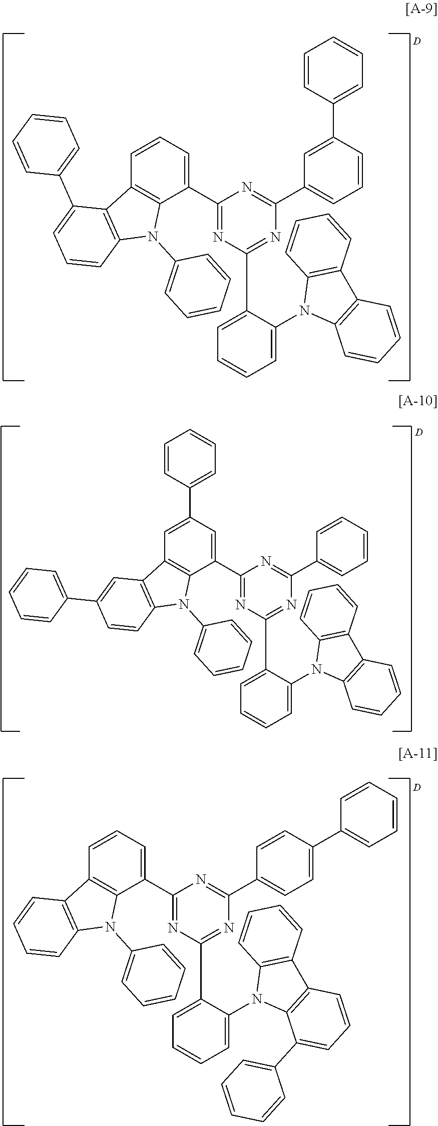 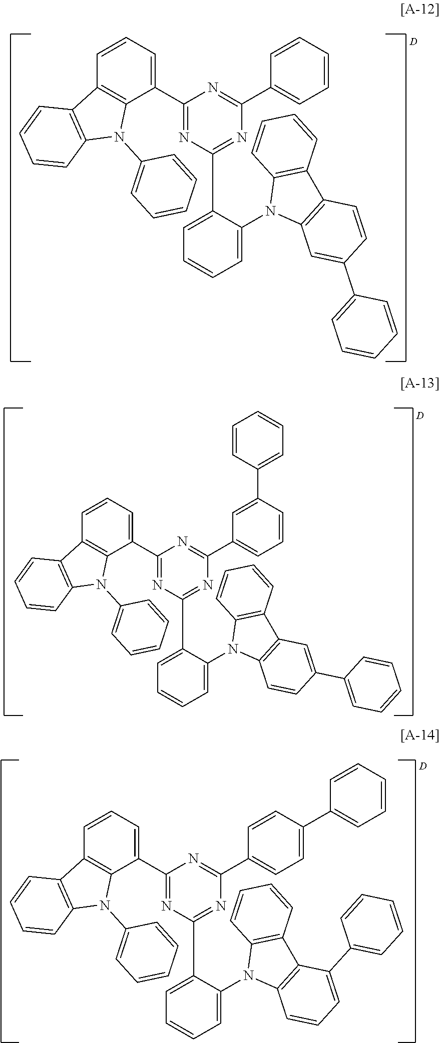 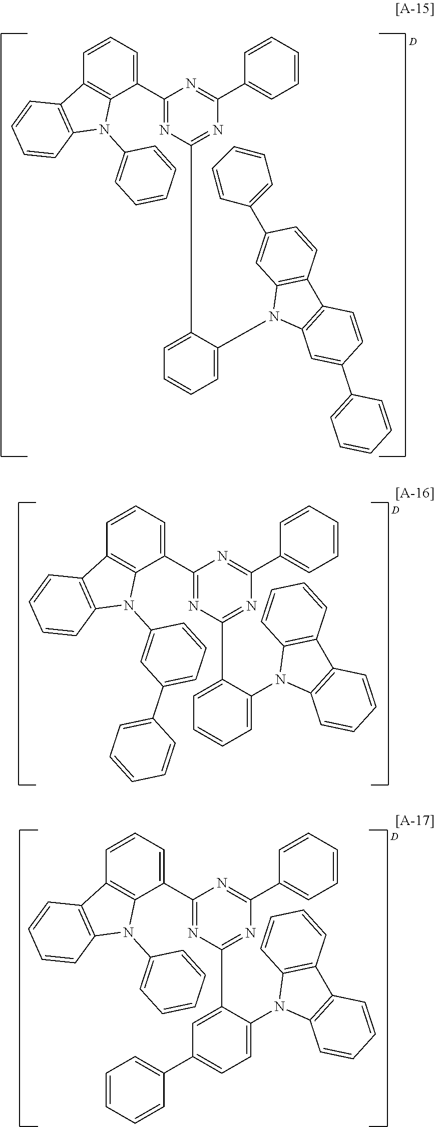 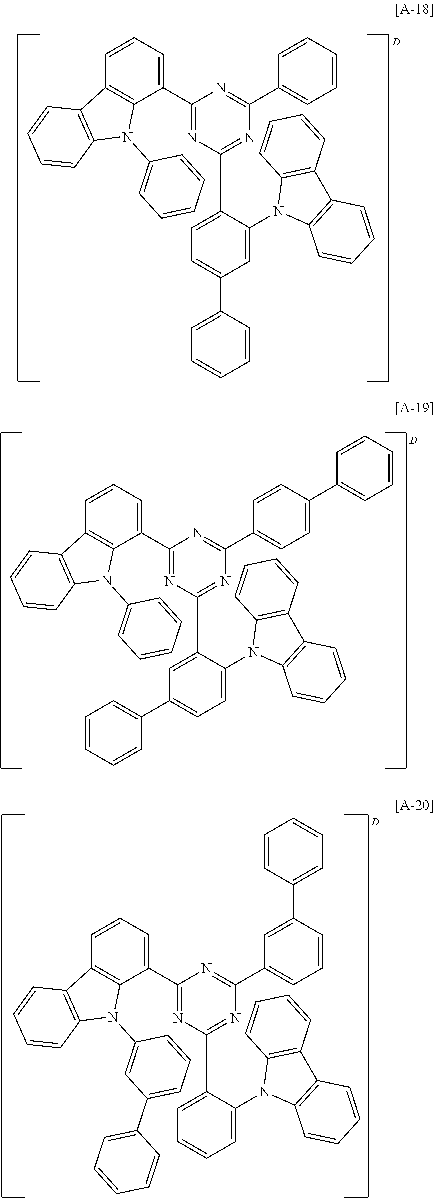 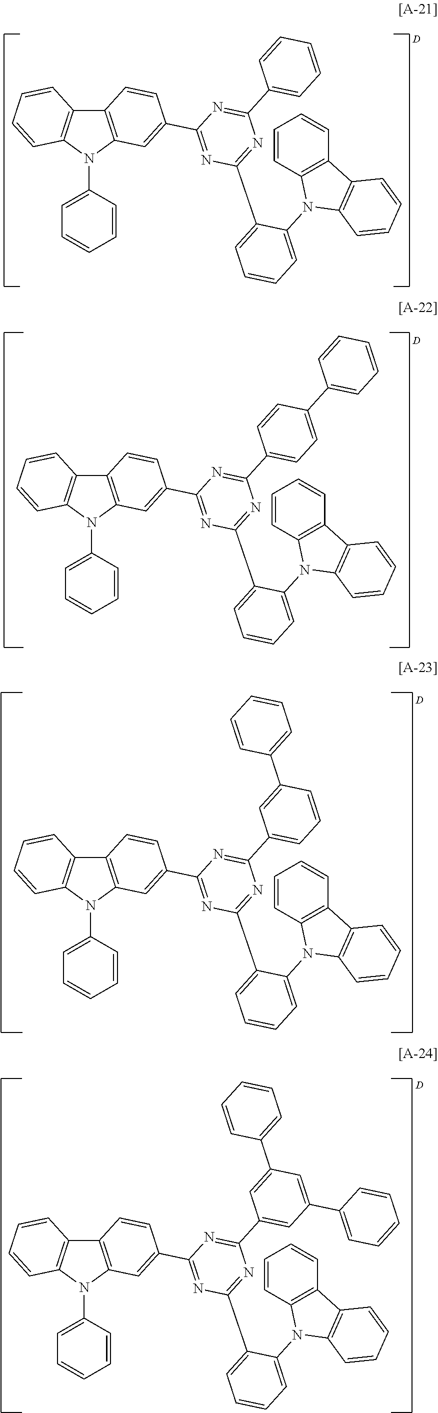 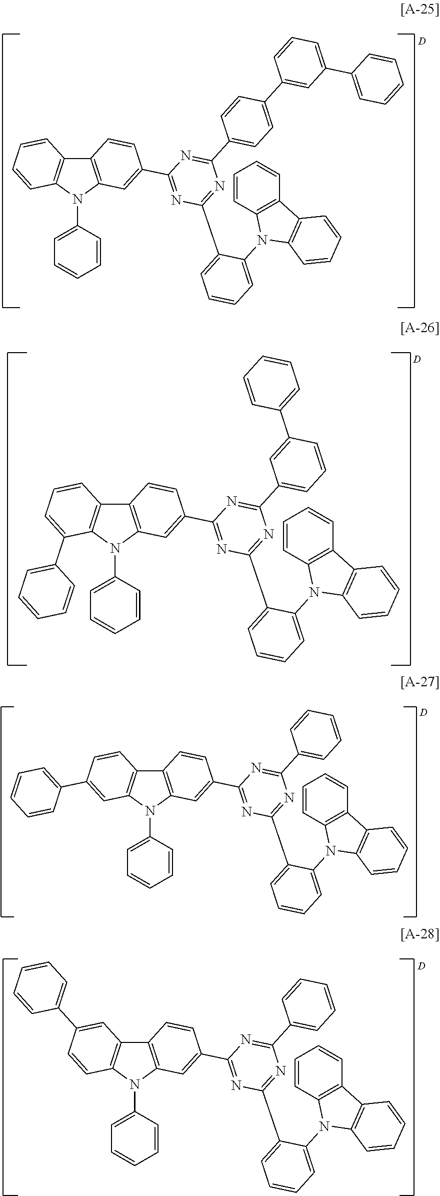 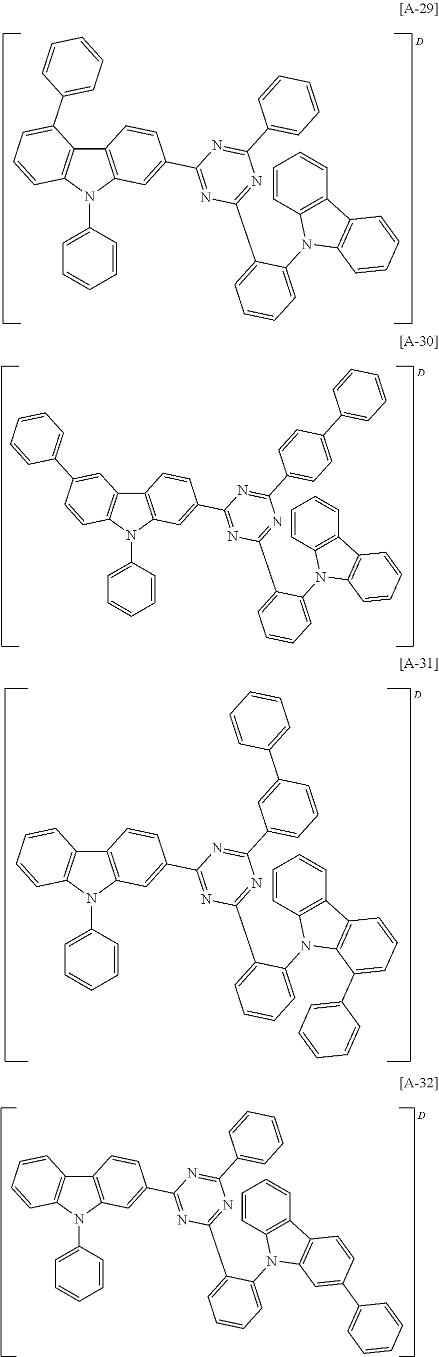 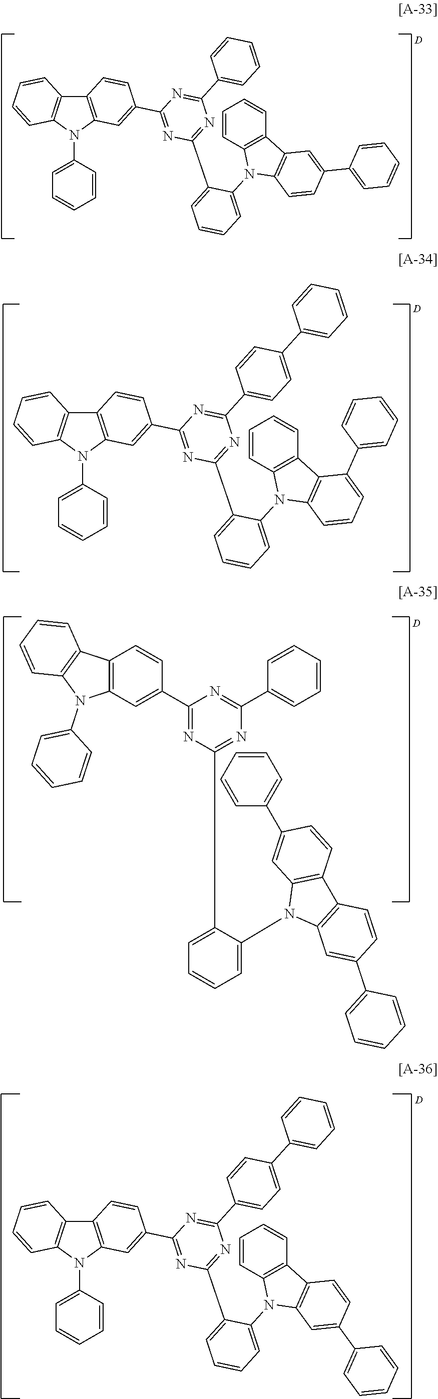 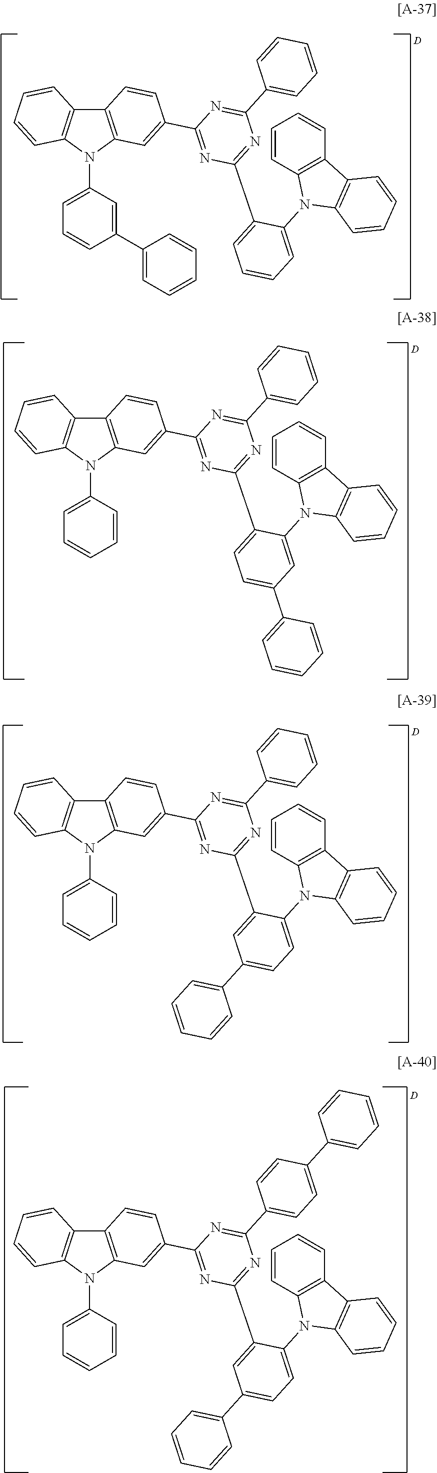 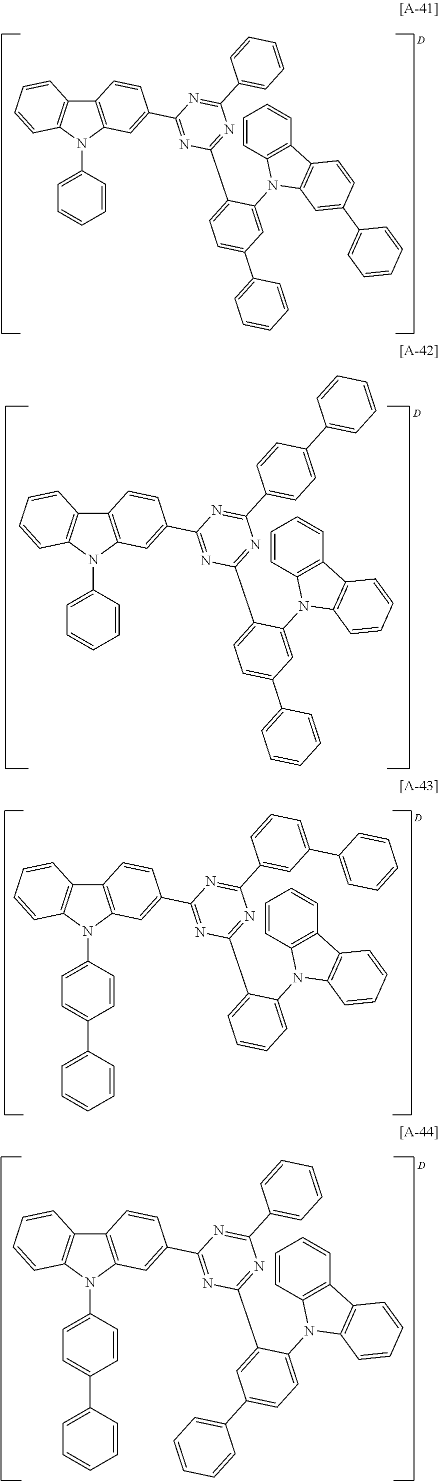 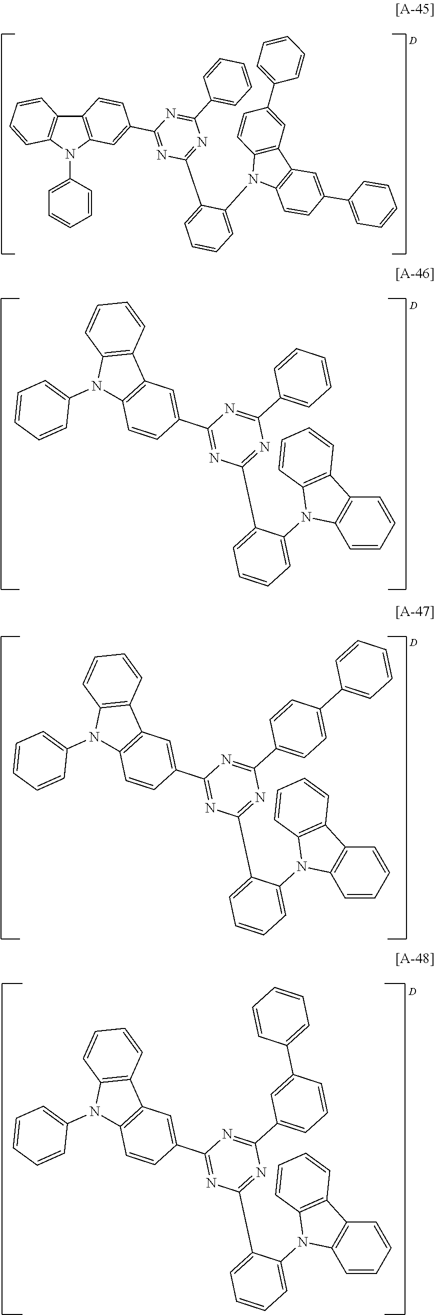 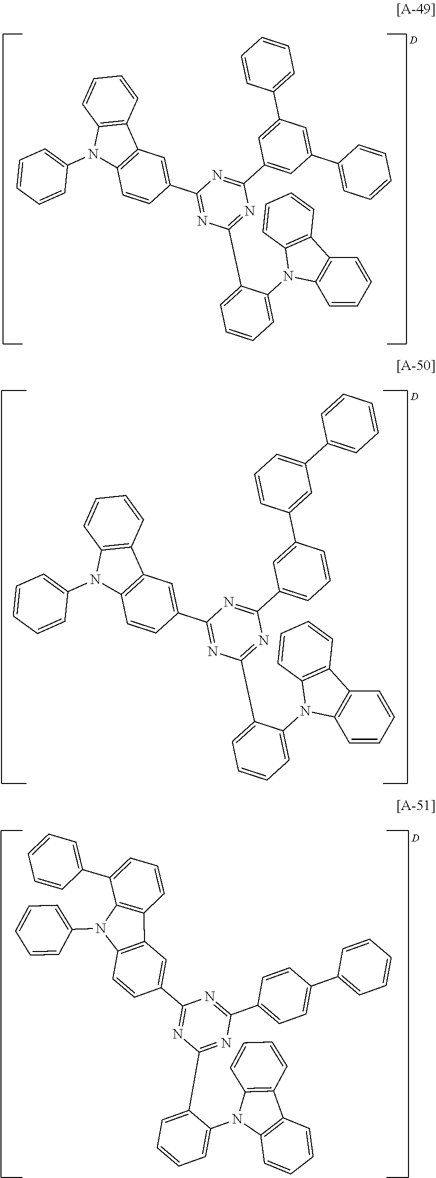 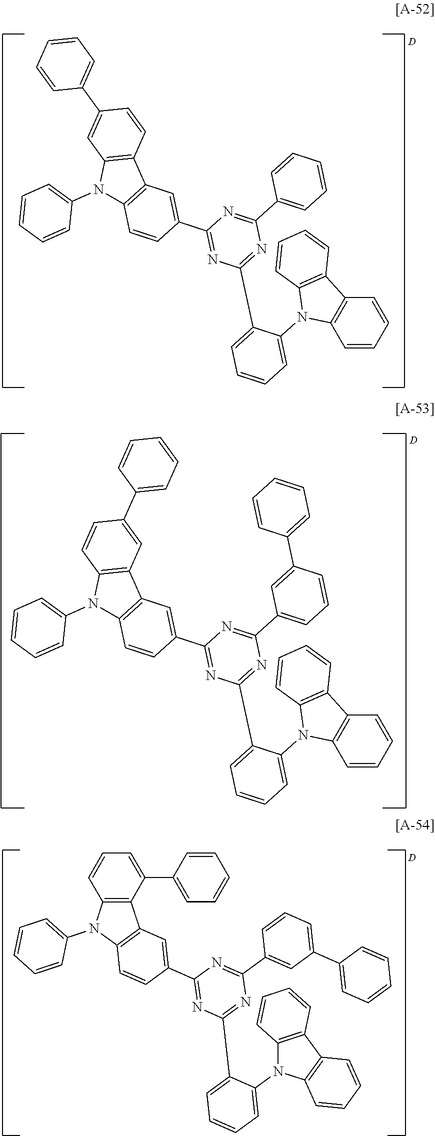 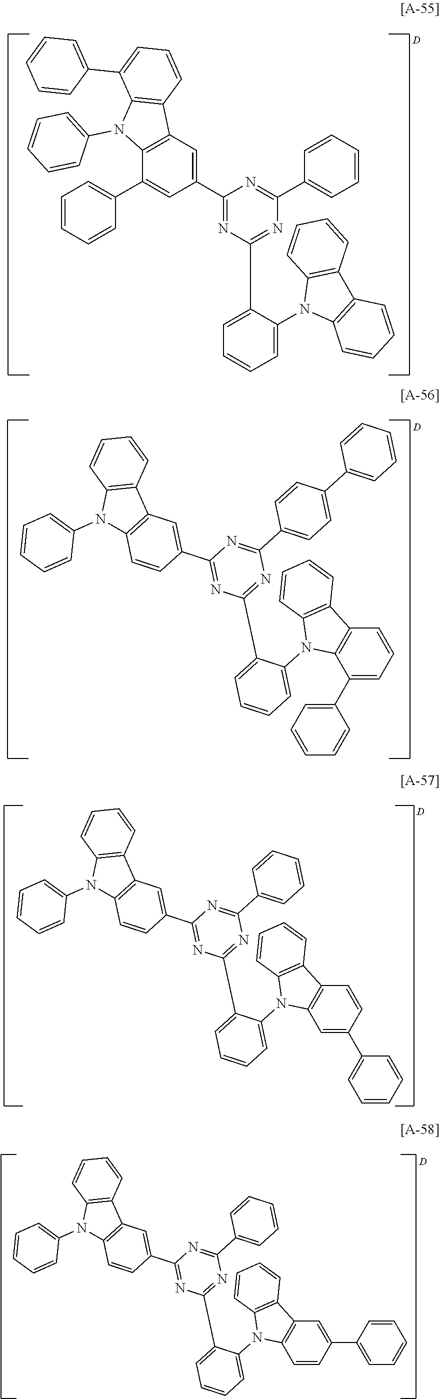 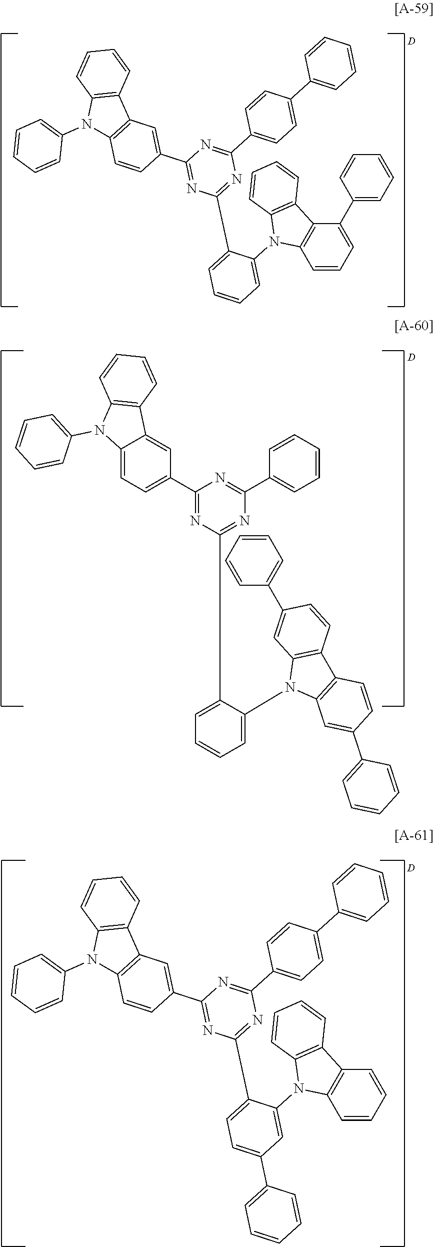 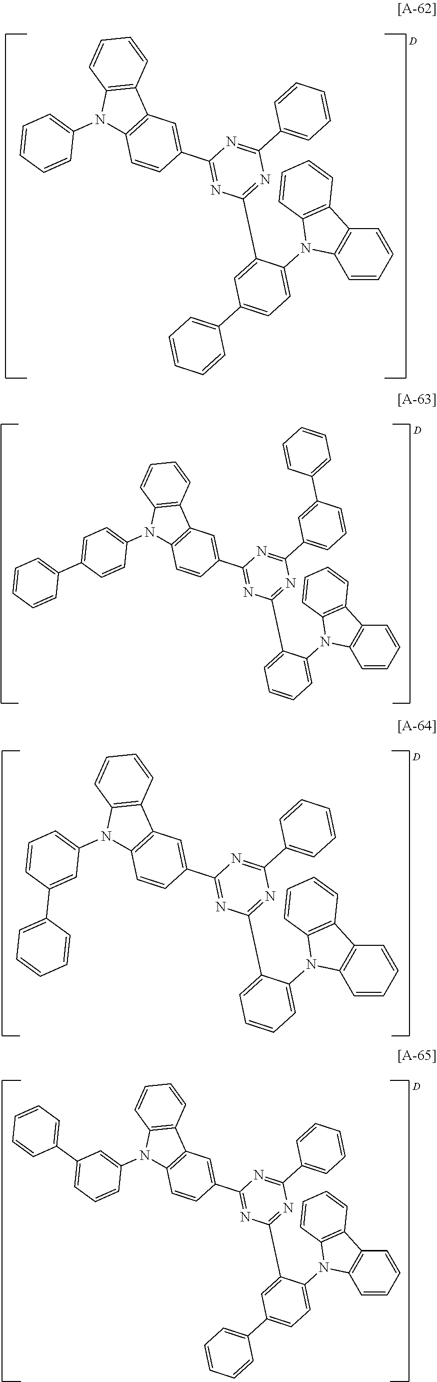 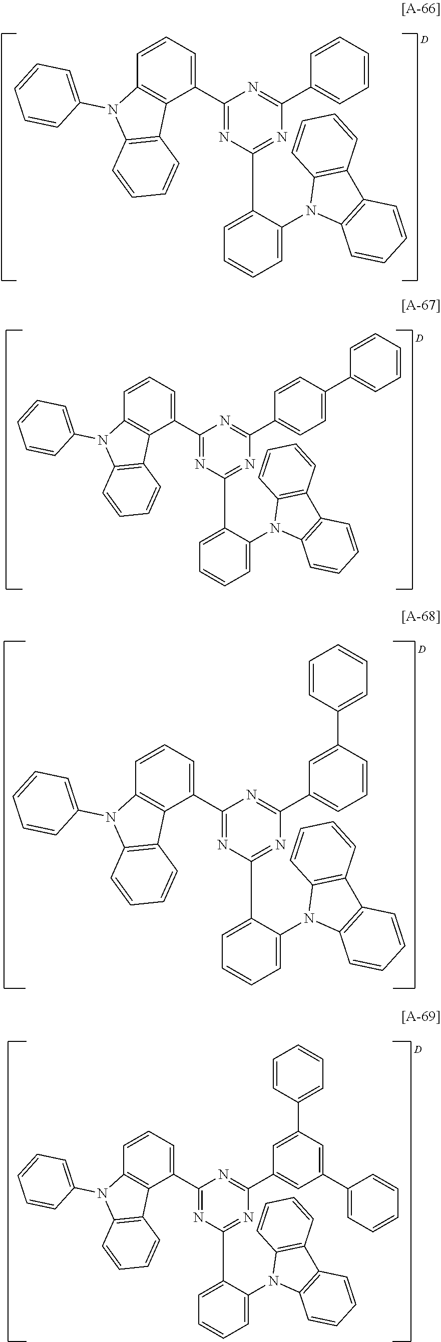 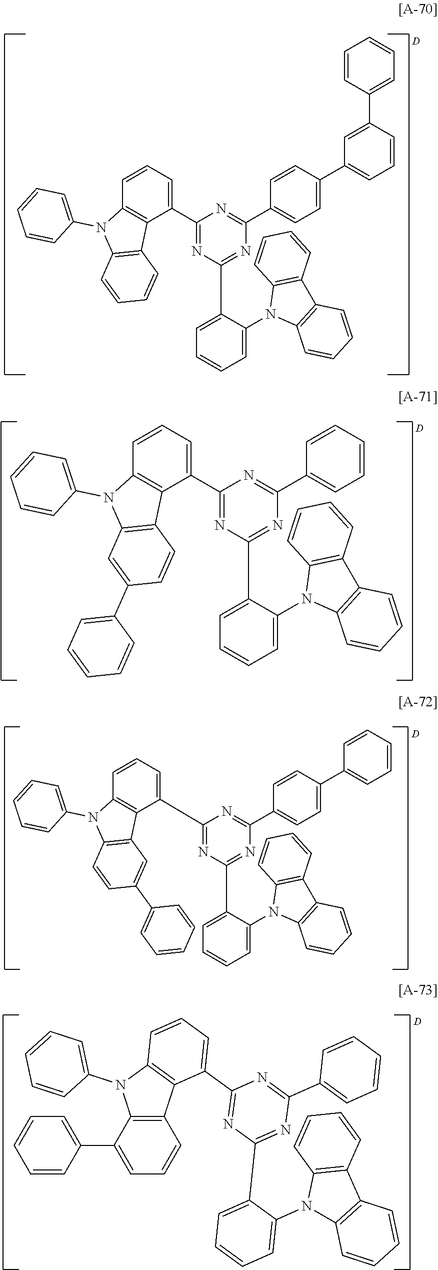 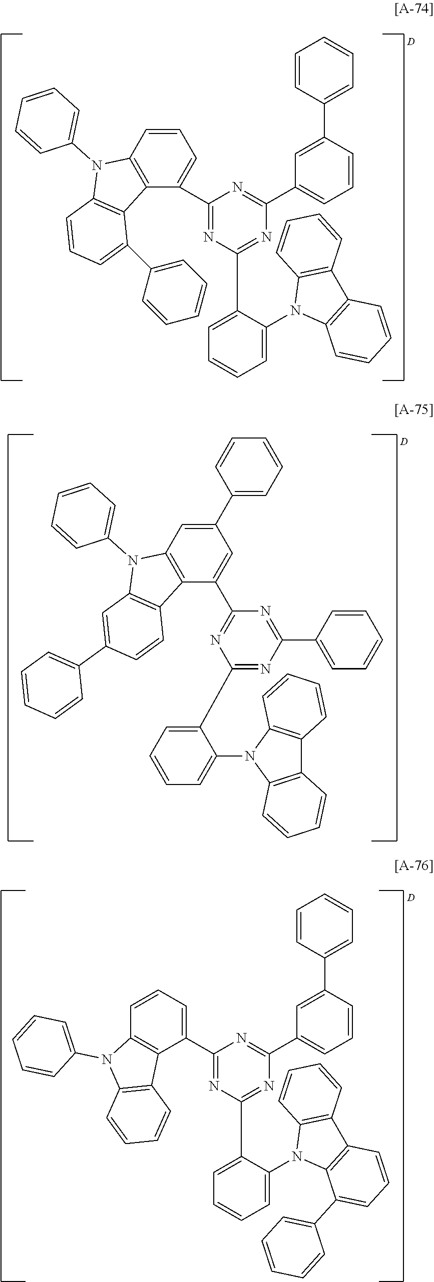 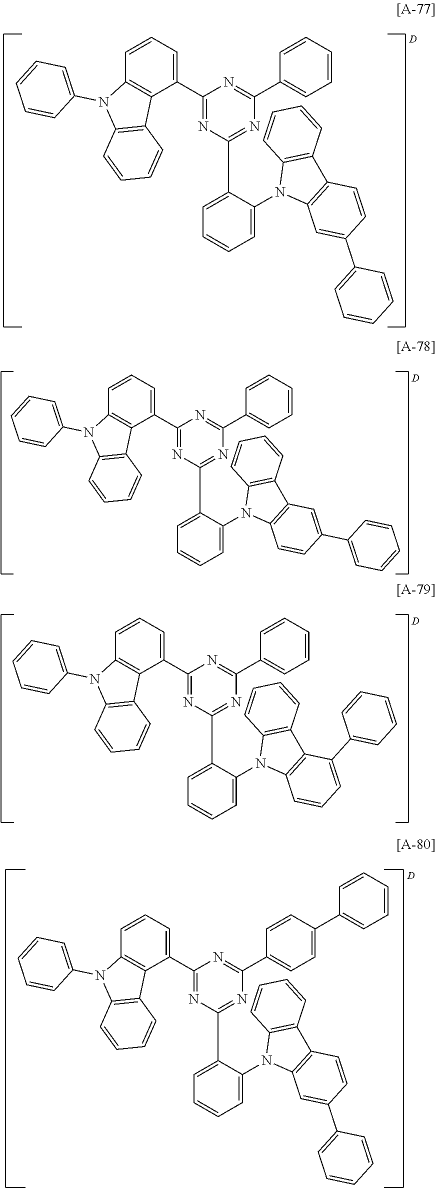 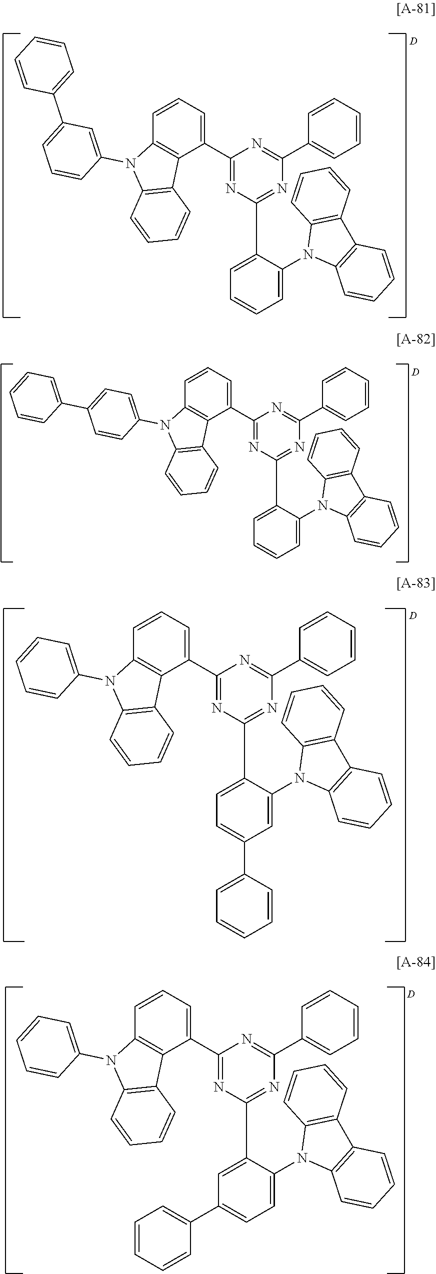 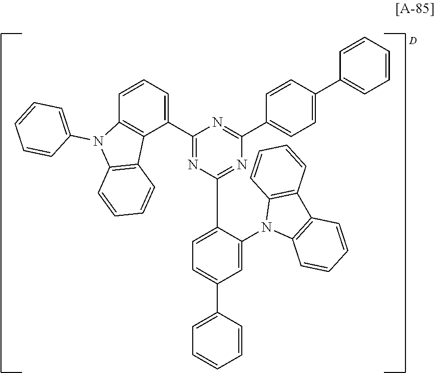
wherein, in Group 1,
D indicates a structure in which deuterium is substituted,
a substitution rate of D is 10% to 100%.

9. An organic optoelectronic device, comprising
an anode and a cathode facing each other, and
at least one organic layer between the anode and the cathode,
wherein the organic layer comprises the compound for the organic optoelectronic device as claimed in claim 1.

10. The organic optoelectronic device as claimed in claim 9, wherein
the organic layer comprises a light emitting layer, and
the light emitting layer comprises the compound for the organic optoelectronic device.

11. A display device comprising the organic optoelectronic device as claimed in claim 9.
